(19) United States
(12) Patent Application Publication
(10) Pub. No.: US 2025/0073218 A1
(43) Pub. Date: Mar. 6, 2025

(54) NLRP3 PROTEIN DEGRADATION INDUCING COMPOUND

(71) Applicant: UPPTHERA, Incheon (KR)

(72) Inventors: Soo Hee Ryu, Incheon (KR); Ji Hoon Ryu, Seoul (KR); Hwa Jin Lee, Incheon (KR); Im Suk Min, Gyeonggi-do (KR); Han Kyu Lee, Gyeonggi-do (KR); Jun Kyu Lee, Gyeonggi-do (KR); Ji Su Lee, Gyeonggi-do (KR); Su Youn Koh, Incheon (KR); Seong Hoon Kim, Incheon (KR)

(73) Assignee: UPPTHERA, Incheon (KR)

(21) Appl. No.: 18/035,033

(22) PCT Filed: Nov. 4, 2021
(86) PCT No.: PCT/KR2021/015860

§ 371 Date: May 2, 2023

(30) Foreign Application Priority Data
Nov. 4, 2020 (KR) 10-2020-0145752
Jul. 20, 2021 (KR) 10-2021-0094952

(51) Int. Cl. A61K 31/454; A61K 31/427; C07D 401/04; C07D 401/14; C07D 417/14

(52) U.S. Cl. CPC A61K 31/454; A61K 31/427; C07D 401/04; C07D 401/14; C07D 417/14

(57) ABSTRACT

The present invention relates to NLRP3 protein degradation inducing compounds. Specifically, the present invention provides a bifunctional compound in which an NLRP3 protein binding moiety and an E3 ubiquitin ligase binding moiety are connected by a chemical linker, a method for preparing the same, a method for degrading NLRP3 protein using the same, and the use for preventing or treating NLRP3 inflammasome-related diseases.

### TECHNICAL FIELD

The present invention relates to NLRP3 protein degradation inducing compounds, a method for preparing the same, and the use for preventing or treating NLRP3 inflammasome-related diseases using the same.

### BACKGROUND ART

NLRP3 (NOD-, LRR- and pyrin domain-containing protein 3) protein is an intracellular sensor protein capable of detecting a wide range of microbial motifs, endogenous danger signals, and environmental stimuli. A structure of NLRP3 protein is composed of an amino-terminal pyrin domain (PYD), a domain present int NAIP, CIITA, HET-E and TP1 (NACHT), and a leucine-rich repeat (LRR) domain, wherein the NACHT domain has ATPase activity essential for the self-assembly and function of NLRP3.

When NLRP3 proteins are stimulated in cells, NLRP3 proteins interact with each other through the NACHT domain in NLRP3 to form oligomers, and ASC proteins are recruited to form an ASC speck, followed by the recruitment of inactive Caspase-1 protein. As a result, when forming an NLRP3 inflammasome, which is a large protein complex composed of NLRP3 protein (sensor function), ASC protein (adaptor function), and Caspase 1 protein (effector function), Caspase-1 which is activated by self-cleavage from inactive Caspase-1 releases potent pro-inflammatory cytokines IL-1P and IL-18 to the outside of the cell, and pyroptosis cell death mediated by Gasdermin D (GSDMD) is induced (document [Swanson et al. Nature Reviews Immunology 19.8 (2019): 477-489.], and the like). As a result, it has been reported that various NLRP3 inflammasome-related diseases such as Alzheimer's disease, multiple sclerosis, atherosclerosis, inflammatory bowel disease, and the like, are induced or exacerbated.

Accordingly, NLRP3 protein has been considered as a drug target for NLRP3 inflammasome-related diseases. In this regard, compounds with various structures, such as MCC950, CY-09, Oridonin, Tranilast, MNS, OLT1177 (dapansutrile), BAY 11-7082, BOT-4-one, Parthenolide, INF39, and the like, have been studied and developed as NLRP3 inflammasome inhibitors.

However, small-molecule compounds developed to date as NLRP3 protein inhibitors have been reported to have limitations in inhibiting the specific enzyme activity in the target protein due to the structural characteristics of the NLRP3 protein, and to have clinical problems in the development process, such as off-target side effects, low blood half-life, hepatotoxicity, and the like (document [Mangan, Matthew S J, et al. Nature reviews Drug discovery 17.8 (2018): 588-606.]).

Therefore, for the development of a therapeutic agent for NLRP3 inflammasome-related diseases, it is required to develop alternative therapeutic drugs to solve the problems in the development of small molecule compounds according to the related art.

### DISCLOSURE

### Technical Problem

An object of the present invention is to provide novel NLRP3 protein degradation inducing compounds.

Another object of the present invention is to provide a method for preparing NLRP3 protein degradation inducing compounds and the use thereof.

### Technical Solution

The present inventors newly synthesized PROTAC (proteolysis-targeting chimera) compounds targeting the degradation of NLRP3 protein, and first discovered that the compounds could be used for the treatment of NLRP3 inflammasome-related diseases by effectively degrading NLRP3 protein in cells. Accordingly, the present invention provides NLRP3 protein degradation inducing compounds, a method for preparing the same, and the use thereof.

### NLRP3 Protein Degradation Inducing Compound

In one aspect, the present invention provides novel compounds that induce degradation of NLRP3 protein. Specifically, the present inventors provide NLRP3 protein targeting degradation inducing compounds utilizing CRBN or VHL E3 ubiquitin ligase.

In an embodiment, the present invention provides a compound represented by the following Formula I, a stereoisomer thereof, or a pharmaceutically acceptable salt thereof:

ULM-Linker-NTM [Formula I]

in Formula I above, NTM is an NLRP3 protein binding moiety, ULM is a CRBN or VHL E3 ubiquitin ligase binding moiety, and Linker is a group chemically linking ULM and NTM.

The present inventors found for the first time that the compound represented by the Formula I could place the NLRP3 protein and the E3 ubiquitin ligase in close proximity within cells, thereby inducing artificial ubiquitination of the NLRP3 protein, and thus degradation of the NLRP3 protein could be achieved by the ubiquitin-proteasome system (UPS) in the cells (FIG. 1).

In other words, the technical feature of the present invention is the first discovery of the NLRP3 protein targeting PROTAC (Proteolysis targeting chimera) compound and usefulness thereof.

The compound represented by Formula I, which is NLRP3 protein targeting PROTAC compound of the present invention, is composed of ULM, NTM, and Linker moieties, and each moiety will be described below.

### (1) NLRP3 Protein Binding Moiety (NTM)

In the compound represented by Formula I of the present invention, NTM is an NLRP3 protein binding moiety.

In the present invention, NLRP3 (NACHT, LRR and PYD domains-containing protein 3) is a protein encoded by NLRP3 gene (Entrez Gene: LRP3 NLR family pyrin domain containing 3 [Homo sapiens (human)], Gene ID: 114548). NLRP3 protein is mainly expressed in immune cells such as macrophages in the body and plays a major role in the innate immune system as a pattern recognition receptor (PRR). NLRP3 protein is known to be involved in various inflammatory-mediated diseases by constituting the NLRP3 inflammasome together with ASC and Caspase-1, and the like.

Compounds capable of inhibiting the activity of the NLRP3 protein by directly binding to the NLRP3 protein are known in the art, for example, reference may be made to the document [El-Sharkawy, Lina Y., David Brough, and Sally Freeman. Molecules 25.23 (2020): 5533.], document [Mangan, Matthew S J, et al. Nature reviews Drug discovery 17.8 (2018): 588-606.], document [Zahid, Ayesha, et al. Frontiers in immunology 10 (2019): 2538.], and the like. Otherwise, Patent documents such as International Patent Publications WO2016/131098, WO2017/184623, WO2018/215818, WO2018/225018, WO2019/008025, WO2019/008029, WO2019/023147, WO2019/034688, WO2019/034690, WO2019/034692, WO2019/034693, WO2019/068772, WO2019/092170, WO2019/092171, WO2019/092172, WO2019/166619, WO2019/166621, WO2019/166623, and the like, may be used as references.

As an example, an inhibitor compound that binds directly to NLRP3 protein may be referred to as having the following structure:

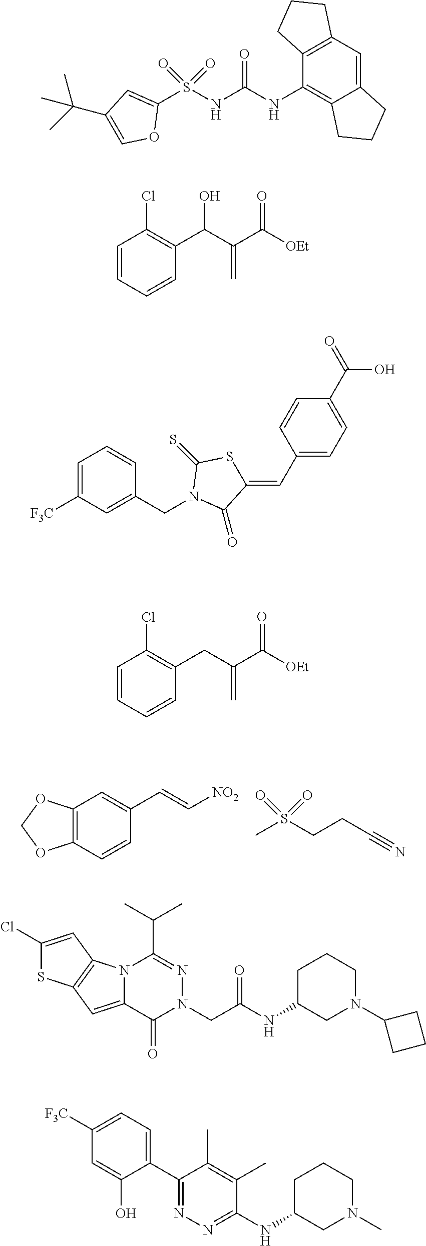

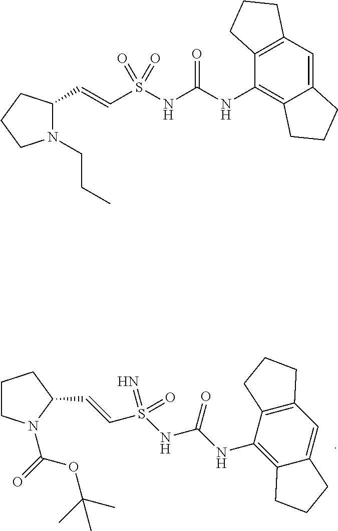 [SMILES: CCCN1CCC[C@@H]1/C=C/S(=O)(=O)NC(=O)Nc1c2c(cc3c1CCC3)CCC2 ; CC(C)(C)OC(=O)N1CCC[C@@H]1/C=C/S(=N)(=O)NC(=O)Nc1c2c(cc3c1CCC3)CCC2]

The compound represented by Formula I of the present invention functions to degrade NLRP3 protein by inducing ubiquitination of the NLRP3 protein in cells through the bifunctionality of the PROTAC compound. The NTM moiety of Formula I of the present invention is functionally distinct from the NLRP3 inhibitor as a single compound, but those skilled in the art, when referring to the specification of the present invention, may appropriately select and synthesize chemical structures capable of being used as the NTM moiety of Formula I of the present invention, based on the NLRP3 protein binding ability according to the NLRP3 inhibitor structure known in the art and the technical idea presented in the present invention. In particular, as of the filing date of the present invention, some structures and mechanisms by which NLRP3 protein inhibitory compounds bind to specific sites of NLRP3 protein are known (PDB code: 7ALV, document [Zahid, Ayesha, et al. Frontiers in immunology 10 (2019): 2538.], document [Swanson et al. Nature Reviews Immunology 19.8 (2019): 477-489.], document [El-Sharkawy et al., Molecules 25.23 (2020): 5533.], document [Hochheiser, Inga V., et al. “Cryo-EM structure of the NLRP3 decamer bound to the cytokine release inhibitory drug CRID3.” bioRxiv (2021)], and International Patent Publication WO2020/208249, and the like).

For example, it has been reported through structural biology research that the MCC950 compound known as a representative inhibitor of the NLRP3 protein is located on the backside of the Walker A motif, which is formed close to the side chains of the two alanines within the NLRP3 protein G₂₂₆AAGIGKT sequence, sandwiched between arginine residues R351 and R578, and comes out on the opposite side, which is formed by the NBD and HD2 domains. As described above, the binding structure of the NLRP3 protein and the NLRP3 binding moiety is known in the art to which the present invention belongs (FIG. 2), and thus those skilled in the art may appropriately select a moiety capable of functioning as the NTM in the Formula I.

In an embodiment, the NTM binds to Walker A and/or B sites present in the NACHT domain of the NLRP3 protein. For example, NTM may bind to the Walker A and/or B sites of the NLRP3 protein by a covalent or non-covalent bond, and may bind thereto in a reversible or irreversible manner.

In an embodiment, the NTM moiety in the compound represented by Formula I of the present invention exhibits a binding activity to NLRP3 protein with an IC₅n of less than about 100, 50, 10, 1, 0.5, 0.1, 0.05, 0.01, 0.005, 0.001 mM, or less than about 100, 50, 10, 1, 0.5, 0.1, 0.05, 0.01, 0.005, 0.001 μM, or less than about 100, 50, 10, 1, 0.5, 0.1, 0.05, 0.01, 0.005, 0.001 nM.

In an embodiment, the NTM moiety of Formula I of the present invention is represented by the following Formula N-1, which shares the parent structure of the diarylsulfonylurea family capable of representing NLRP3 protein binding moieties:

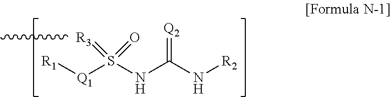

in Formula N-1 above,
R₁ and R₂ are each independently an optionally substitutable cycloalkyl, heterocyclyl, aryl or heteroaryl;
R₃ is O or NR₄;
R₄ is hydrogen, halogen, OH, NH₃, NO₂, CN, C₁₋₆alkyl, C₁₋₆alkoxy, C₂₋₆alkenyl, C₂₋₆alkynyl, 3-10 membered cycloalkyl, 4-10 membered heterocyclyl, 6-10 membered aryl or 4-10 membered heteroaryl; or R₄ and R₁ together with the atoms to which they are attached form an optionally substitutable 5 to 10 membered heterocyclic ring;
Q₁ is a single bond,-NQₓ-, -CQₓQ_{y}-, —CH₂—NQₓ- or —CH₂—CQₓQ_{y}- {wherein Qₓ and Q_{y} are each independently hydrogen or C₁₋₆alkyl};
Q₂ is O or S, and
[symbol] represents that any one hydrogen in the Formula N-1 is substituted with a single bond and connected to the Linker by a covalent bond.

As an example, the NLRP3 protein binding compound having the structure of Formula N-1 above is as follows, and preparation methods thereof and NLRP3 protein binding ability are known in International Patent Publications WO2020/035464, WO2019/034696, WO2020/035464, WO2019/034697, and the like.

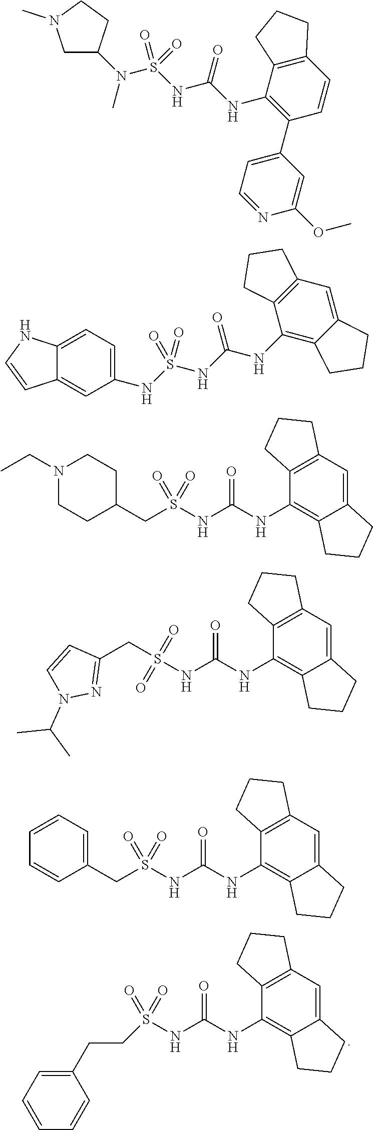

In the compound represented by Formula I of the present invention, the Formula N-1 moiety is covalently linked to the Linker via [symbol] . Here, any hydrogen of the N-1 moiety structure may be substituted with a single bond to be connected to the Linker. For example, the N-1 moiety may be connected to the Linker by substituting hydrogen of the R₁ group in the N-1 moiety with a single bond.

In a more specific exemplary embodiment, the Formula N-1 above is represented by the following Formula N-2:

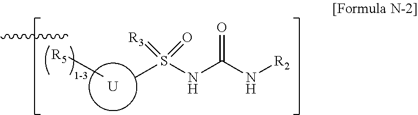

in Formula N-2 above,
\textcircled{U} is 4-10 membered cycloalkyl, 4-10 membered heterocyclyl, 6-10 membered aryl or 4-10 membered heteroaryl {wherein the heterocyclyl or heteroaryl contains 1 to 3 N, S or O atoms};
R₂ is

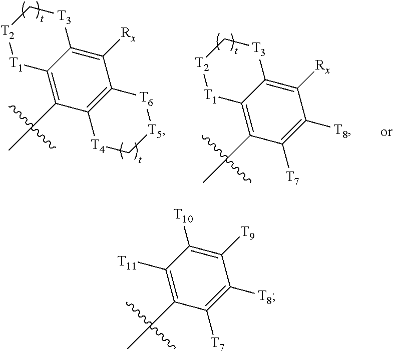

t is 0 or 1;

T₁ to T₆ are each independently —CH₂—, —NH—, —S—, —SO₂— or —O— {wherein hydrogen in T₁ to T₆ may each independently be substituted with C₁₋₆alkyl, halogen, OH, OCH₃, NH₂ or CN};
T₇ to T₁₁ are each independently C₁₋₆alkyl, halogen, OH, OCH₃, CN, 4-10 membered cycloalkyl, 4-10 membered heterocyclyl, 6-10 membered aryl or 4-10 membered heteroaryl {wherein the heterocyclyl or heteroaryl contains 1 to 3 N, S or O atoms};
hydrogen in T₇ to T₁₁ may each independently be substituted with C₁₋₆alkyl, halogen, OH, OCH₃, NH₂ or CN;
Rₓ is hydrogen, halogen, C₁₋₆alkyl, O(C₁₋₄alkyl), OH, CN, NH₃, NO₂, SO₂ or CN;
R₃ is O or NR₄;
R₄ is hydrogen, halogen, OH, OCH₃, CN or C₁₋₃alkyl;
R₅ is each independently —(C₀₋₄alkylene)-R₆, —(C₀₋₄alkylene)-R_{L1}-(C₀₋₄alkylene)-R₆ or —(C₀₋₄alkylene)-R_{L1}—(C₀₋₄alkylene)-R_{L2}-(C₀₋₄alkylene)-R₆;
R₆ is hydrogen, halogen, OH, OCH₃, COH, COOH, CN, NH₂, NH(C₁₋₃alkyl), NCH₃(C₁₋₃alkyl), SO₂, C₁₋₆alkyl, C₂₋₆alkenyl, C₂₋₆alkynyl, cycloalkyl, heterocyclyl, aryl or heteroaryl {wherein at least one hydrogen in R₆ may each independently be substituted with halogen, C₁₋₃alkyl, C₁₋₃alkoxy, OH, NH₂, NO₂ or CN};
R_{L1} and R_{L2} are each independently —O—, —CO—, —COO—, —OCO—, —NH—, —N(C₁₋₃alkyl)-, —NHCO—, —N(C₁₋₃alkyl)CO—, —CONH—, —CON(C₁₋₃alkyl)- or —NHCONH—; and
[symbol] represents that any one of hydrogens in the Formula N-2 is substituted with a single bond and connected to the Linker by a covalent bond.

As an example, NLRP3 protein binding compounds having the structure of Formula N-2 above are shown as follows, and preparation methods thereof and NLRP3 protein binding ability are known in International Patent Publications WO2017/184623, WO2019/068772, WO2019/008029, WO2019/034688, WO2019/034693, WO2019/092172, and the like.

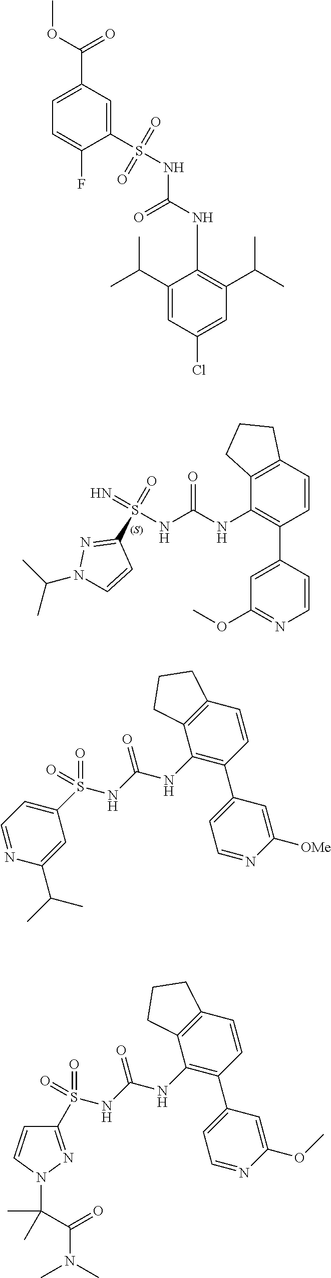

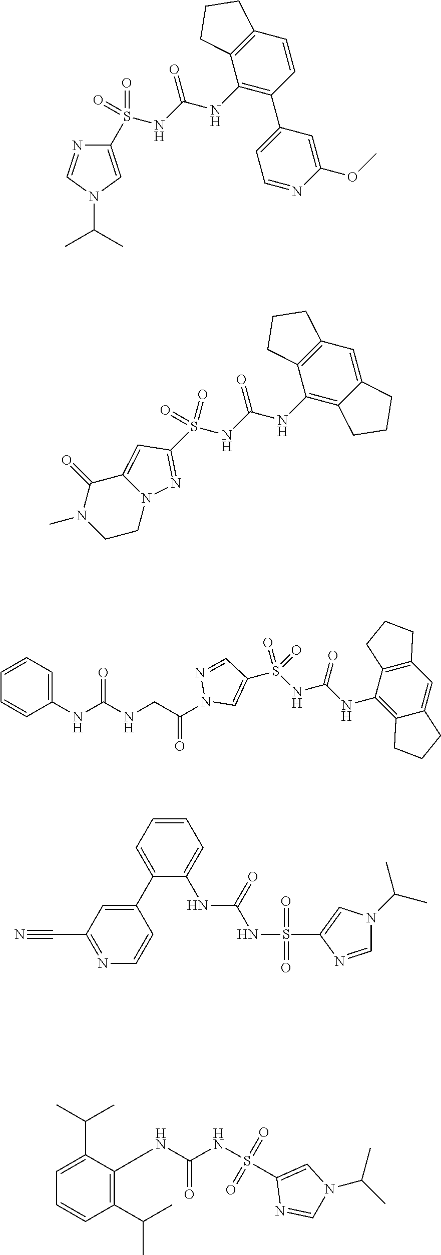

As in Formula N-1, the Formula N-2 moiety is covalently linked to the Linker via [symbol] . Here, any hydrogen of the N-2 moiety structure may be substituted with a single bond to be connected to the Linker. For example, the N-2 moiety may be connected to the Linker by substituting hydrogen of the one R₅ group in the N-2 moiety with a single bond.

In a more specific exemplary embodiment, the Formula N-2 above is represented by the following Formula N-3:

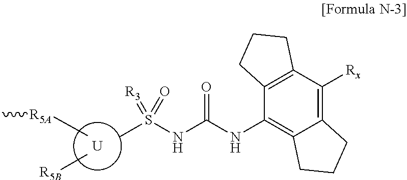

in Formula N-3 above,
R₃ is O, NH or N—CN;

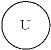

is 5-7 membered cycloalkyl, 5-7 membered heterocyclyl, phenyl or 5-6 membered heteroaryl {wherein the heterocyclyl or heteroaryl contains 1 to 3 N, S or O atoms};
R_{5A} and R_{5B} are each independently —(C₀₋₄alkylene)-R₆ or (C₀₋₄alkylene)-R_{L}—(C₀₋₄alkylene)-R₆;
R₆ is hydrogen, halogen, OH, OCH₃, COH, COOH, CN, NH₂, NH(C₁₋₃alkyl), NCH₃(C₁₋₃alkyl), SO₂, C₁₋₆alkyl, C₂₋₆alkenyl, C₂₋₆alkynyl, 4-8 membered cycloalkyl, 4-8 membered heterocyclyl, phenyl or 5-6 membered heteroaryl {wherein the heterocyclyl or heteroaryl contains 1 to 3 N, S or O atoms, and hydrogens in R₆ may be substituted with C₁₋₃alkyl, OH, —O(C₁₋₃alkyl), CN, halogen};
R_{L} is —O—, —CO—, —COO—, —OCO—, —NH—, —N(C₁₋₃alkyl)-, —NHCO—, —N(C₁₋₃alkyl)CO—, —CONH—, —CON(C₁₋₃alkyl)- or —NHCONH— and
Rₓ is hydrogen, halogen, OH or CN, and
[symbol] represents that any one hydrogen of R_{5A} is substituted with a single bond to be covalently connected to the Linker.

As an example, the NLRP3 protein binding compound having the structure of Formula N-3 above is as follows, and preparation methods thereof and NLRP3 protein binding ability are known in document [El-Sharkawy, Lina Y., David Brough, and Sally Freeman. Molecules 25.23 (2020): 5533.], and the like.

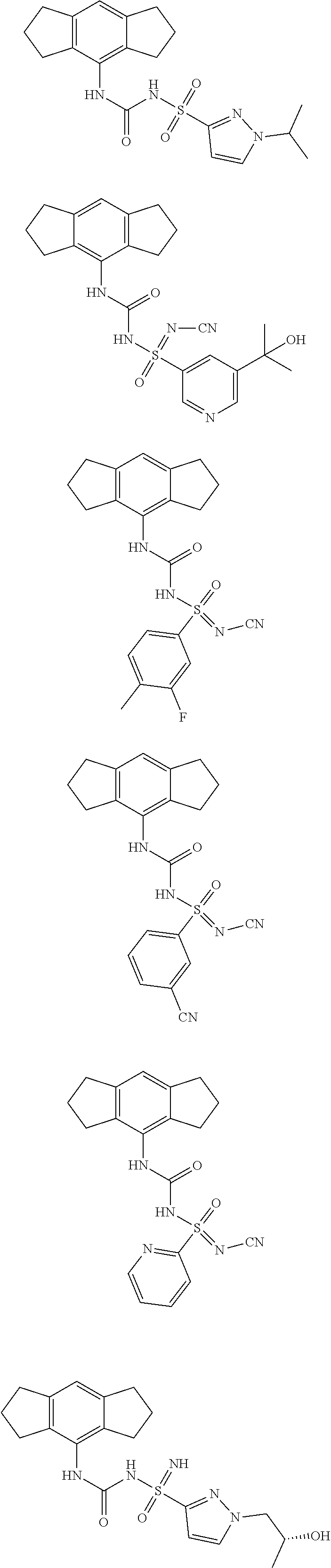

As still another example, the NLRP3 protein binding compound having the structure of Formula N-3 above is as follows, and preparation methods thereof and NLRP3 protein binding ability are known in International Patent Publications WO2016/131098, WO2017/184623, WO2018/215818, WO2018/225018, WO2019/008025, WO2019/008029, WO2019/023147, WO2019/034688, WO2019/034690, WO2019/034692, WO2019/034693, WO2019/068772, WO2019/092170, WO2019/092171, WO2019/092172, WO2019/166619, WO2019/166621, WO2019/166623, and the like.

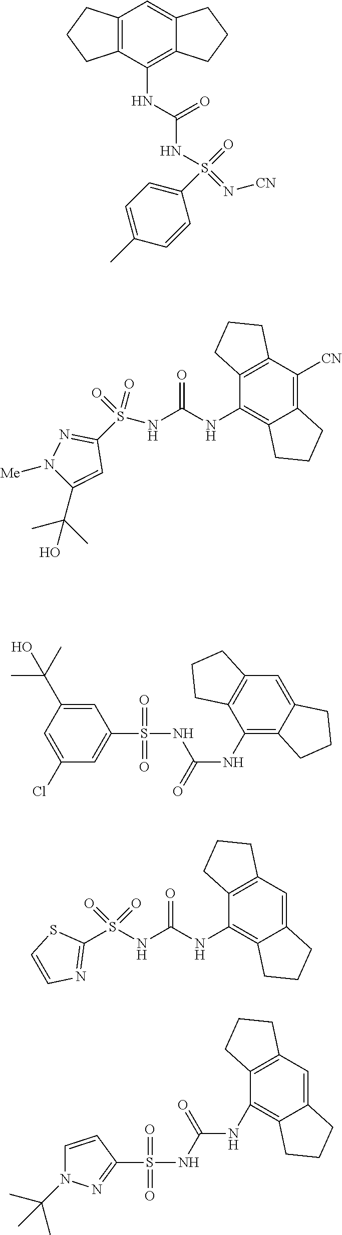 [SMILES: Cc1ccc(S(=O)(=NC#N)NC(=O)Nc2c3c(cc4c2CCC4)CCC3)cc1 ; Cn1nc(S(=O)(=O)NC(=O)Nc2c3c(c(C#N)c4c2CCC4)CCC3)cc1C(C)(C)O ; CC(C)(O)c1cc(Cl)cc(S(=O)(=O)NC(=O)Nc2c3c(cc4c2CCC4)CCC3)c1 ; O=C(Nc1c2c(cc3c1CCC3)CCC2)NS(=O)(=O)c1nccs1 ; CC(C)(C)n1ccc(S(=O)(=O)NC(=O)Nc2c3c(cc4c2CCC4)CCC3)n1]

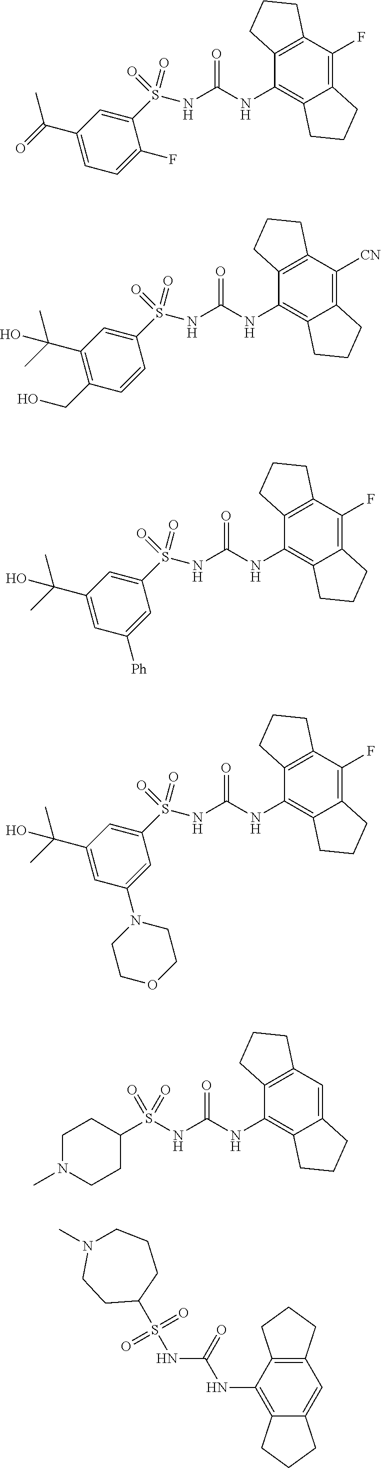 [SMILES: CC(=O)c1ccc(F)c(S(=O)(=O)NC(=O)Nc2c3c(c(F)c4c2CCC4)CCC3)c1 ; CC(C)(O)c1cc(S(=O)(=O)NC(=O)Nc2c3c(c(C#N)c4c2CCC4)CCC3)ccc1CO ; CC(C)(O)c1cc(-c2ccccc2)cc(S(=O)(=O)NC(=O)Nc2c3c(c(F)c4c2CCC4)CCC3)c1 ; CC(C)(O)c1cc(N2CCOCC2)cc(S(=O)(=O)NC(=O)Nc2c3c(c(F)c4c2CCC4)CCC3)c1 ; CN1CCC(S(=O)(=O)NC(=O)Nc2c3c(cc4c2CCC4)CCC3)CC1 ; CN1CCCC(S(=O)(=O)NC(=O)Nc2c3c(cc4c2CCC4)CCC3)CC1]

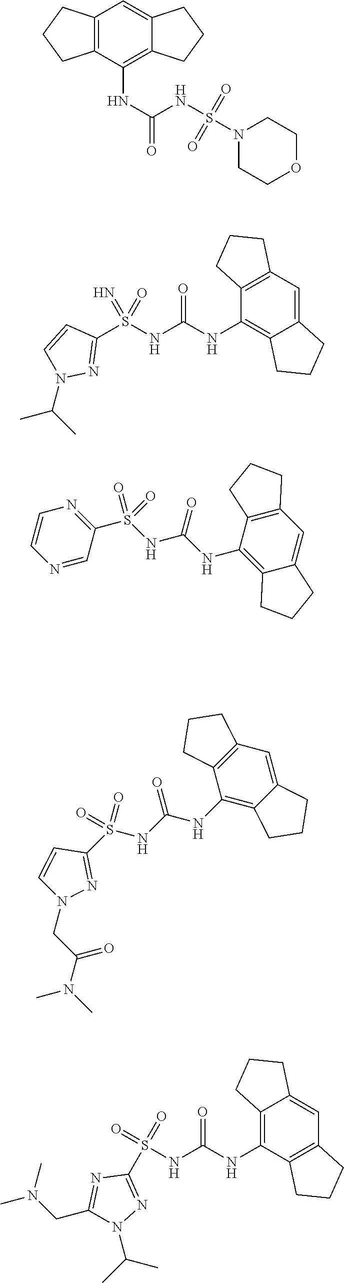 [SMILES: O=C(Nc1c2c(cc3c1CCC3)CCC2)NS(=O)(=O)N1CCOCC1 ; CC(C)n1ccc(S(=N)(=O)NC(=O)Nc2c3c(cc4c2CCC4)CCC3)n1 ; O=C(Nc1c2c(cc3c1CCC3)CCC2)NS(=O)(=O)c1cnccn1 ; CN(C)C(=O)Cn1ccc(S(=O)(=O)NC(=O)Nc2c3c(cc4c2CCC4)CCC3)n1 ; CC(C)n1nc(S(=O)(=O)NC(=O)Nc2c3c(cc4c2CCC4)CCC3)nc1CN(C)C]

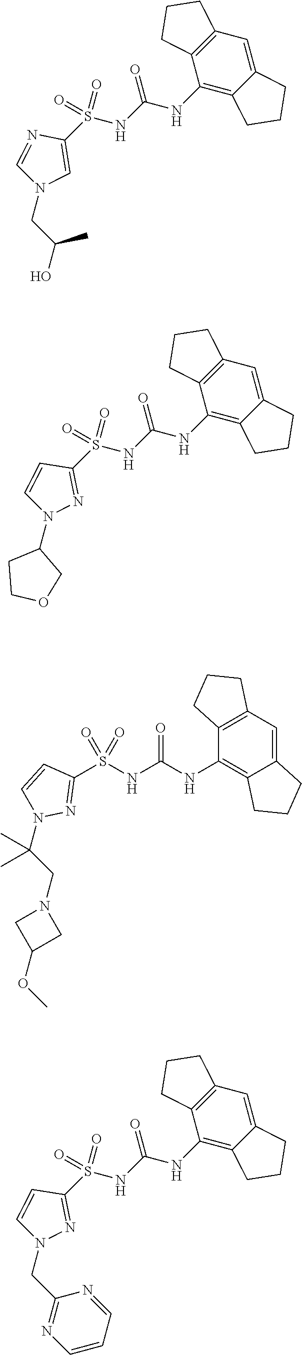 [SMILES: C[C@@H](O)Cn1cnc(S(=O)(=O)NC(=O)Nc2c3c(cc4c2CCC4)CCC3)c1 ; O=C(Nc1c2c(cc3c1CCC3)CCC2)NS(=O)(=O)c1ccn(C2CCOC2)n1 ; COC1CN(CC(C)(C)n2ccc(S(=O)(=O)NC(=O)Nc3c4c(cc5c3CCC5)CCC4)n2)C1 ; O=C(Nc1c2c(cc3c1CCC3)CCC2)NS(=O)(=O)c1ccn(Cc2ncccn2)n1]

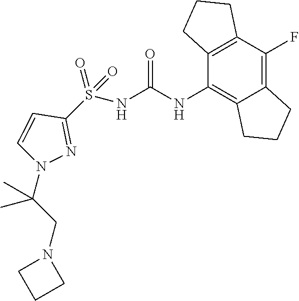 [SMILES: CC(C)(CN1CCC1)n1ccc(S(=O)(=O)NC(=O)Nc2c3c(c(F)c4c2CCC4)CCC3)n1]

As still another example, the NLRP3 protein binding compound having the structure of Formula N-3 above is as follows, and preparation methods thereof and NLRP3 protein binding ability are described in Examples and Experimental Examples to be described below.

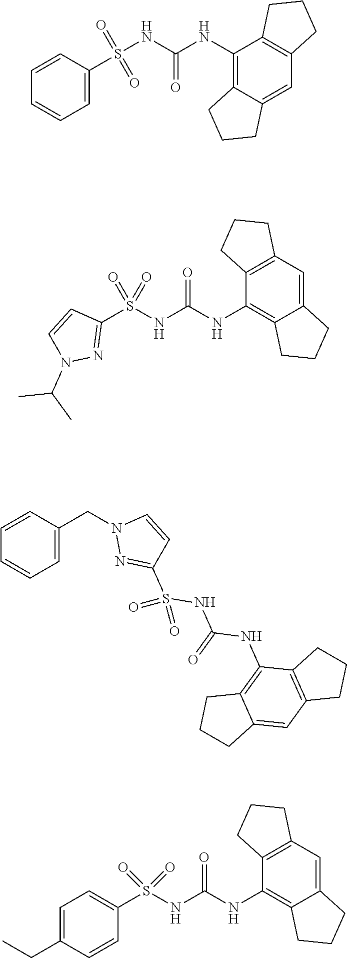

In Formula N-3, [symbol] represents that any one hydrogen of R_{5A} is substituted with a single bond and connected to the Linker by a covalent bond.

### (2) CRBN or VHL E3 Ubiquitin Ligase Binding Moiety (ULM)

In the present invention, E3 ubiquitin ligase is a protein that promotes the ubiquitin transfer to a target substrate protein, and ULM in the compound represented by Formula I according to the present invention is a moiety capable of binding to CRBN or VHL E3 ubiquitin ligase.

In the present invention, CRBN refers to Cereblon E3 ubiquitin ligase. CRBN together with DDB1, Cu14A, and ROC1 constitute an E3 ubiquitin ligase complex, wherein CRBN is a substrate recognition subunit of the complex. Some compounds capable of binding to CRBN E3 ubiquitin ligase are known in the art.

For example, since it was known that thalidomide binds to the CRBN E3 ubiquitin ligase (document [Ito et al. 2010]), a number of imide-based small molecule compounds (immunomodulatory imide drug; IMiD) including lenalidomide and pomalidomide have been reported to have CRBN binding ability (document [Chamberlain and Brian. 2019], document [Akuffo et al. 2018]), document [Burslem et al. 2018], and the like). In addition, the binding structure of thalidomide, which is a representative CRBN E3 ubiquitin ligase binding material, and the CRBN E3 ubiquitin ligase is known in the art as shown in FIG. 3 (PDB code: 4CI1), which allows the skilled person to appropriately select a structure capable of functioning as the CRBN E3 ubiquitin ligase binding moiety (ULM) in Formula I.

In an embodiment, the CRBN E3 ubiquitin ligase binding moiety of the present invention is a compound represented by the following Formula A-1:

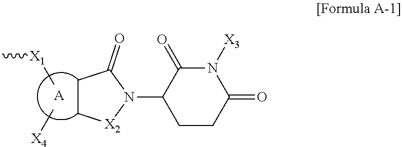

in Formula A-1 above,

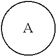

is a ring selected from the group consisting of

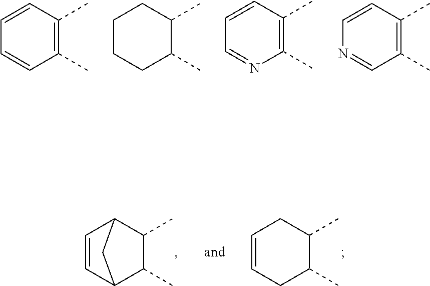

X₁ is a single bond, —CH₂—, —NH—, —O—, —CH₂CH₂—, —CC— —CO—, —COO—, —NHCO— or —CONH—;
X₂ is —CH₂—, —CH(C₁₋₄alkyl)-, —NH—, —N(C₁₋₄alkyl)-, —O—, —CO—, —CH₂—CH₂—, —NH—CH₂—, —NH—CH(C₁₋₄alkyl)-, —N═CH—, —N═C(C₁₋₄alkyl)- or —N═N—;
X₃ is hydrogen or C₁₋₄alkyl; and
X₄ is hydrogen, halogen, C₁₋₆alkyl, CN, NH₂, NO₂, OH, COH, COOH or CF₃.

In an embodiment, the Formula A-1 above is represented by the following Formula A-2:

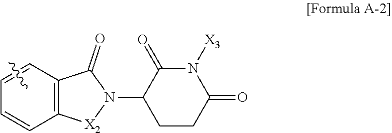

in Formula A-2 above,
X₂ is —CH₂—, —CH(C₁₋₄alkyl)-, —CO— or —N═N—; and
X₃ is hydrogen or C₁₋₃alkyl.

In an embodiment of the present invention, the Formula A-2 above may be selected from the group consisting of the following moieties:

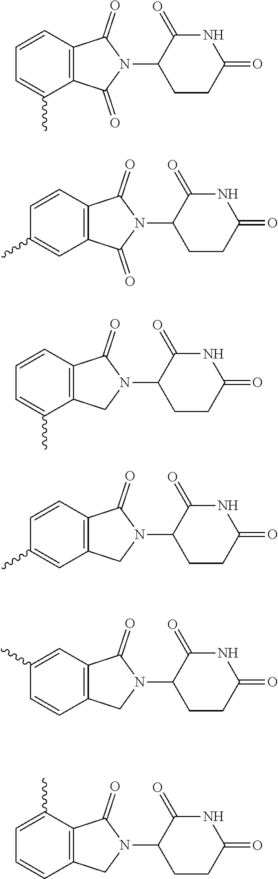

An example of the CRBN E3 ubiquitin ligase binding moiety according to the present invention is as follows (document [Chamberlain and Brian. 2019] and document [Akuffo et al. 2018]).

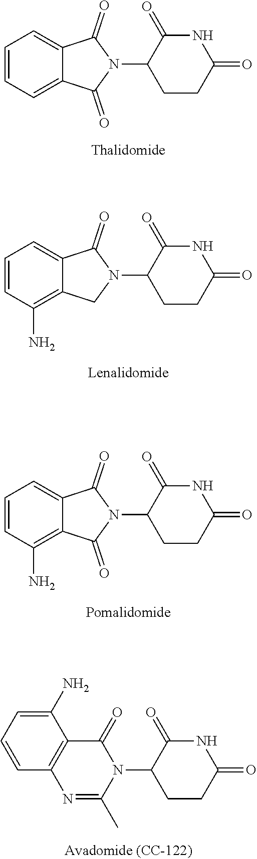 [SMILES: O=C1CCC(N2C(=O)c3ccccc3C2=O)C(=O)N1 ; Nc1cccc2c1CN(C1CCC(=O)NC1=O)C2=O ; Nc1cccc2c1C(=O)N(C1CCC(=O)NC1=O)C2=O ; Cc1nc2cccc(N)c2c(=O)n1C1CCC(=O)NC1=O]

Another example of the CRBN E3 ubiquitin ligase binding moiety according to the present invention is as follows (document [Burslem et al. 2018]).

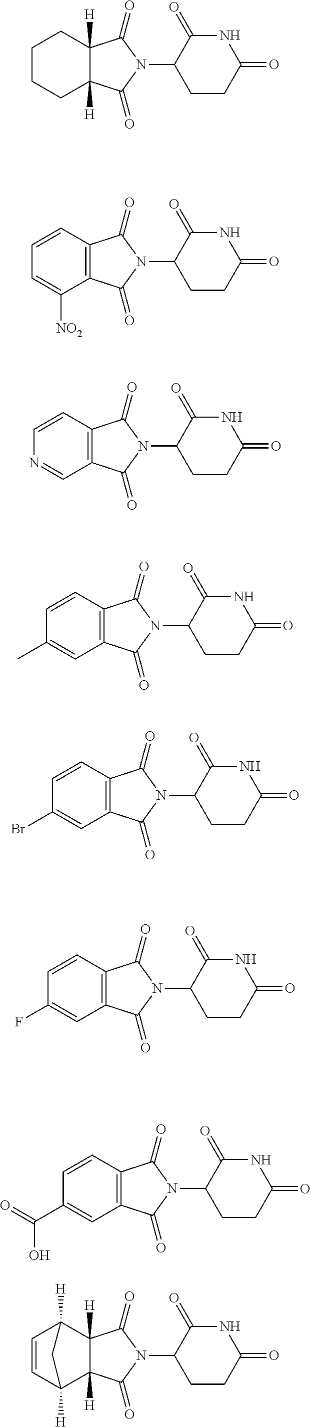

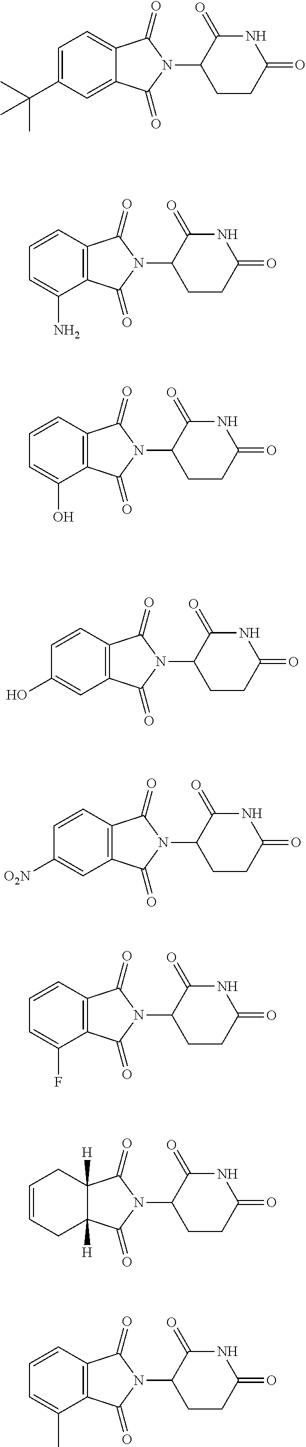

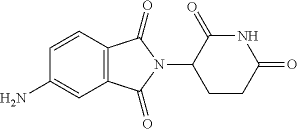 [SMILES: Nc1ccc2c(c1)C(=O)N(C1CCC(=O)NC1=O)C2=O]

According to still another embodiment of the invention, the ULM is a VHL E3 ubiquitin ligase binding moiety.

In the present invention, VHL means von Hippel-Lindau tumor suppressor. VHL together with Elongin B, Elongin C, CUL2, and Rbx1 constitutes a VCB E3 ubiquitin ligase complex, wherein VHL is a substrate recognition subunit of the complex. Some compounds capable of binding to VHL E3 ubiquitin ligase are known in the art.

For example, since peptides such as Ala-Leu-Ala-(Hy)Pro-Tyr-Ile-Pro heptapeptide (document [Schneekloth et al. 2004]) and Leu-Ala-(Hy)Pro-Tyr-Ile pentapeptide (document [Rodriguez-Gonzalez et al. 2008]) were known, the improved small-molecule VHL E3 ubiquitin ligase binding compounds have been reported (document [Buckley et al. J. Am. Chem. Soc. 2012], document [Buckley et al. Ang. Chem. Int. Ed. 2012], document [Galdeano et al. 2014], document [Soares et al. 2017], and the like). In addition, the binding structure of the moiety that binds to the VHL E3 ubiquitin ligase is known in the art as shown in FIG. 4 (PDB code: 4W9L), which allows the skilled person to appropriately select a structure capable of functioning as the VHL E3 ubiquitin ligase binding moiety (ULM) in Formula I.

In an embodiment, the VHL E3 ubiquitin ligase binding moiety is a compound represented by the following Formula B-1:

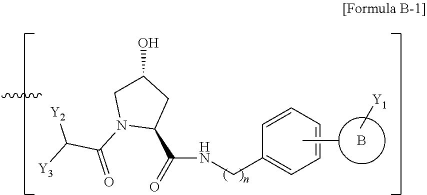

in Formula B-1 above,
n is an integer from 1 to 3;

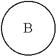

is 5-6 membered cycloalkyl, phenyl, 5-6 membered heterocyclyl or 5-6 membered heteroaryl {wherein the heterocyclyl or heteroaryl contains 1 to 3 N, O or S atoms};
Y₁ is hydrogen or C₁₋₄alkyl;
Y₂ is C₁₋₄alkyl, hydroxy (C₁₋₄alkyl), —(C₀₋₂alkyl)-COH, C₃₋₈cycloalkyl, or phenyl;
Y₃ is hydrogen or

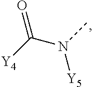

Y₄ is hydrogen, halogen, C₁₋₄alkyl, —O(C₁₋₄alkyl), C₃₋₆cycloalkyl or 4-6 membered heterocyclyl [wherein Y₄ may be substituted with halogen, —OH, —CN, —NHCOH, —NHCOCH₃, —COH, or —COCH₃]; and
Y₅ is hydrogen or C₁₋₄alkyl.

In an embodiment, the VHL E3 ubiquitin ligase binding moiety is a compound represented by the following Formula B-2:

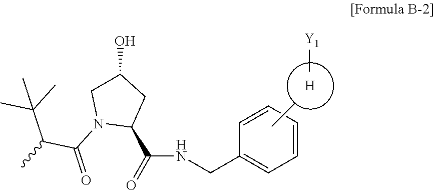

in Formula B-2 above,

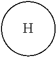

is a 5-membered heteroaryl ring selected from the group consisting of oxazole, isoxazole, thiazole, isothiazole, imidazole, pyrazole, triazole, oxadiazole, pyrrole, pyrrolidine, furan, dihydrofuran, and tetrahydrofuran; and
Y₁ is hydrogen or C₁₋₃alkyl.

Still another example of the VHL E3 ubiquitin ligase binding moiety according to the present invention is as follows (document [Galdeano et al. (2014)]).

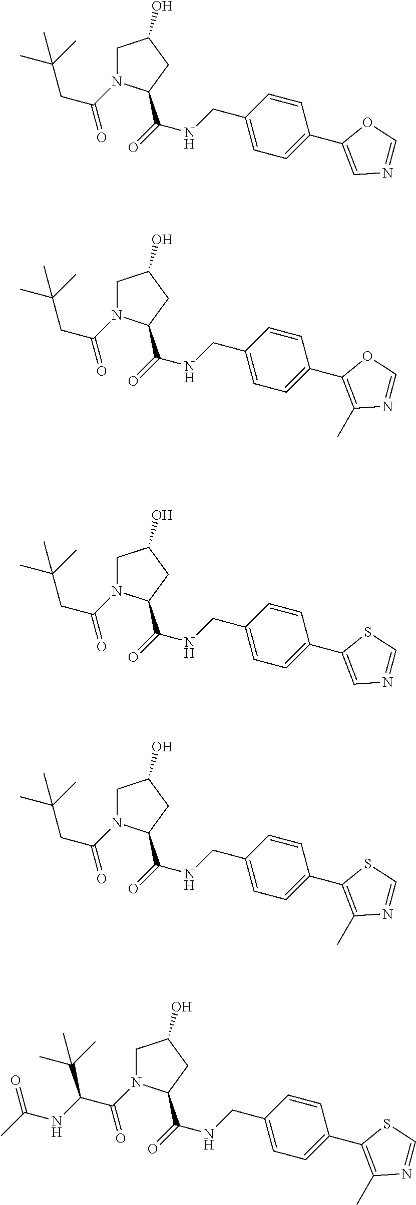 [SMILES: CC(C)(C)CC(=O)N1C[C@H](O)C[C@H]1C(=O)NCc1ccc(-c2cnco2)cc1 ; Cc1ncoc1-c1ccc(CNC(=O)[C@@H]2C[C@@H](O)CN2C(=O)CC(C)(C)C)cc1 ; CC(C)(C)CC(=O)N1C[C@H](O)C[C@H]1C(=O)NCc1ccc(-c2cncs2)cc1 ; Cc1ncsc1-c1ccc(CNC(=O)[C@@H]2C[C@@H](O)CN2C(=O)CC(C)(C)C)cc1 ; CC(=O)N[C@H](C(=O)N1C[C@H](O)C[C@H]1C(=O)NCc1ccc(-c2scnc2C)cc1)C(C)(C)C]

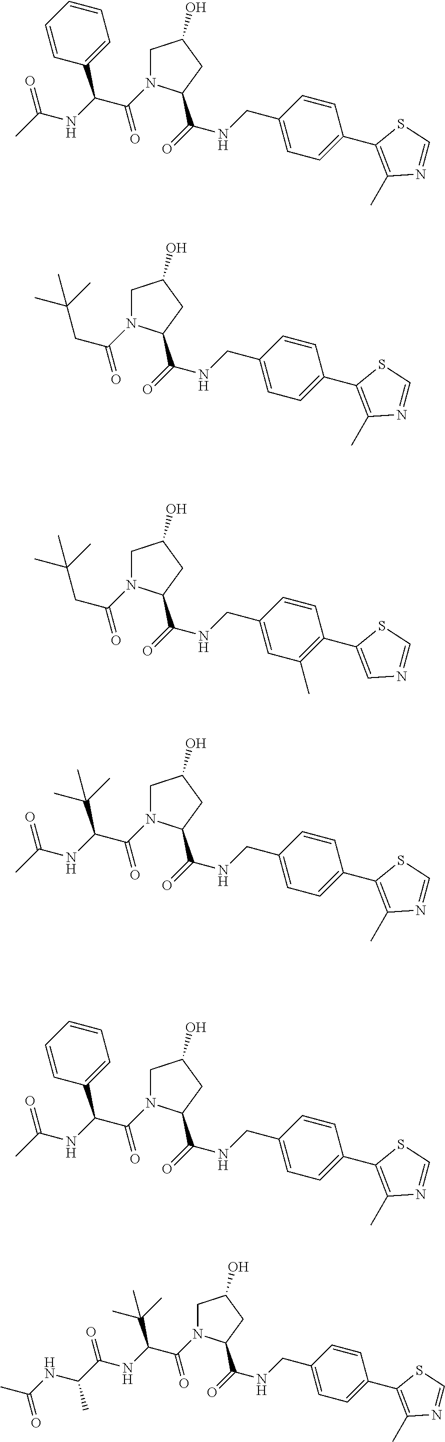 [SMILES: CC(=O)N[C@H](C(=O)N1C[C@H](O)C[C@H]1C(=O)NCc1ccc(-c2scnc2C)cc1)c1ccccc1 ; Cc1ncsc1-c1ccc(CNC(=O)[C@@H]2C[C@@H](O)CN2C(=O)CC(C)(C)C)cc1 ; Cc1cc(CNC(=O)[C@@H]2C[C@@H](O)CN2C(=O)CC(C)(C)C)ccc1-c1cncs1 ; CC(=O)N[C@H](C(=O)N1C[C@H](O)C[C@H]1C(=O)NCc1ccc(-c2scnc2C)cc1)C(C)(C)C ; CC(=O)N[C@H](C(=O)N1C[C@H](O)C[C@H]1C(=O)NCc1ccc(-c2scnc2C)cc1)c1ccccc1 ; CC(=O)N[C@@H](C)C(=O)N[C@H](C(=O)N1C[C@H](O)C[C@H]1C(=O)NCc1ccc(-c2scnc2C)cc1)C(C)(C)C]

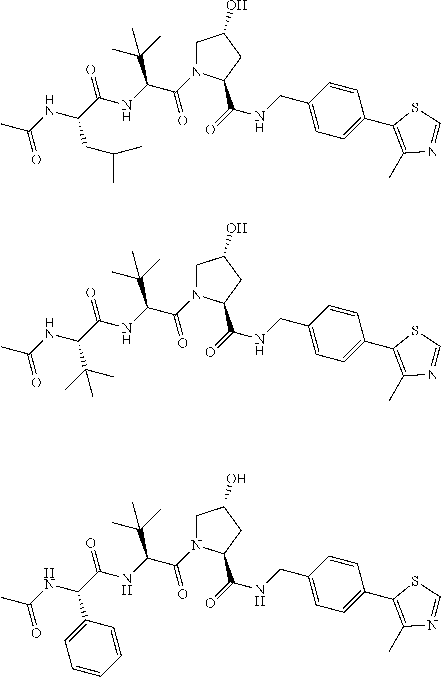 [SMILES: CC(=O)N[C@@H](CC(C)C)C(=O)N[C@H](C(=O)N1C[C@H](O)C[C@H]1C(=O)NCc1ccc(-c2scnc2C)cc1)C(C)(C)C ; CC(=O)N[C@H](C(=O)N[C@H](C(=O)N1C[C@H](O)C[C@H]1C(=O)NCc1ccc(-c2scnc2C)cc1)C(C)(C)C)C(C)(C)C ; CC(=O)N[C@H](C(=O)N[C@H](C(=O)N1C[C@H](O)C[C@H]1C(=O)NCc1ccc(-c2scnc2C)cc1)C(C)(C)C)c1ccccc1]

Still another example of the VHL E3 ubiquitin ligase binding moiety according to the present invention is as follows (document [Soares et al. 2017]).

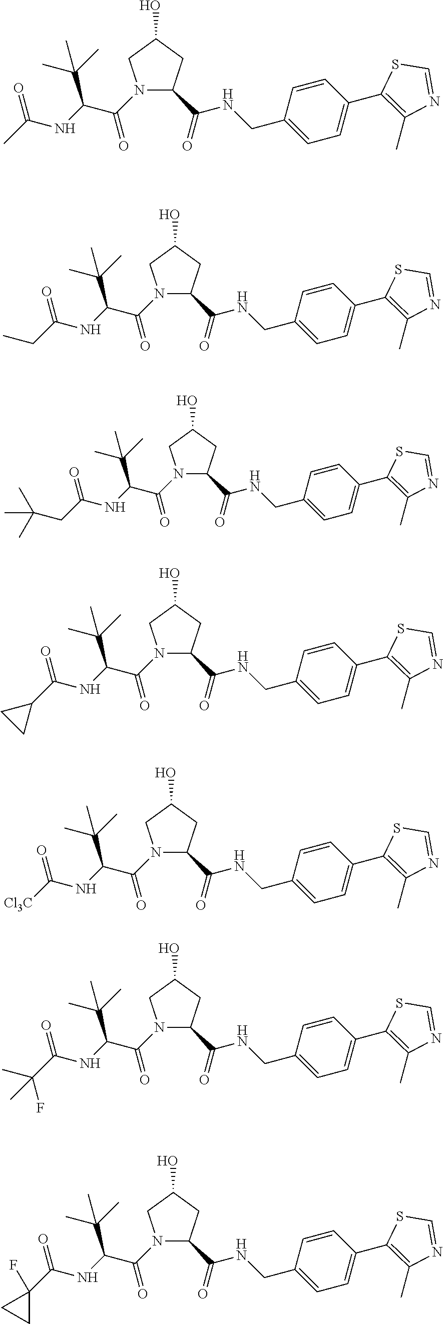

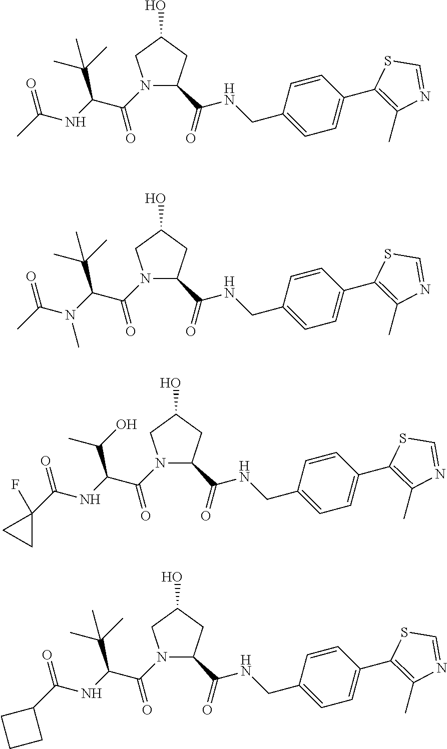 [SMILES: CC(=O)N[C@H](C(=O)N1C[C@H](O)C[C@H]1C(=O)NCc1ccc(-c2scnc2C)cc1)C(C)(C)C ; CC(=O)N(C)[C@H](C(=O)N1C[C@H](O)C[C@H]1C(=O)NCc1ccc(-c2scnc2C)cc1)C(C)(C)C ; Cc1ncsc1-c1ccc(CNC(=O)[C@@H]2C[C@@H](O)CN2C(=O)[C@@H](NC(=O)C2(F)CC2)C(C)O)cc1 ; Cc1ncsc1-c1ccc(CNC(=O)[C@@H]2C[C@@H](O)CN2C(=O)[C@@H](NC(=O)C2CCC2)C(C)(C)C)cc1]

### (3) Linker

In the compound represented by Formula I of the present invention, Linker is a group that chemically connects the ULM and NTM moieties. Through the Linker, the E3 ubiquitin ligase protein targeted by the ULM moiety and the NLRP3 protein targeted by the NTM moiety may interact with each other within an appropriate physical distance, thereby inducing ubiquitination of the target NLRP3 protein. Accordingly, the Linker is included in the present invention without limitation as long as it is a chemical group in the form in which the compound represented by Formula I of the present invention is capable of performing the function of PROTAC compounds that induce ubiquitination of the target NLRP3 protein.

In an embodiment, the Linker is represented by the following Formula L:

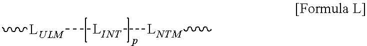

in Formula L above, [symbol] and [symbol] are bonds,
L_{ULM} binds to the ULM moiety through [symbol] linked thereto,
L_{NTM} binds to the NTM moiety through [symbol] linked thereto,
L_{ULM}, L_{NTM}, and L_{INT} are each independently selected from the group consisting of a single bond, —CH₂—, —NH—, —O—, —S—, —SO—, —SO₂—, —CO—, —CH₂CH₂—, —CHCH—, —CC—, —CH₂CH₂O—, —OCH₂CH₂—, —CH₂CH₂S—, —SCH₂CH₂—, —COO—, —CONH—, —NHCO—, and

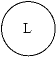

{wherein

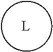

is cycloalkyl, heterocyclyl, aryl or heteroaryl};
L_{ULM}, L_{NTM}, and L_{INT} may each independently be substituted with at least one C₁₋₆alkyl, C₃₋₈cycloalkyl, halogen, hydroxy, amine, nitro, cyano or haloalkyl; and
p is an integer from 1 to 30.

In an embodiment, p is an integer of 1 or more, 5 or more, 10 or more, 15 or more, 20 or more, or 25 or more, and is an integer of 25 or less, 20 or less, 15 or less, 10 or less, or 5 or less.

In the present invention, L_{ULM} may be

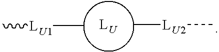

{wherein L_{U1} is selected from the group consisting of a single bond, —CH₂—, —CH₂CH₂—, —CH═CH—, —CC—, —NH—, —NCH₃—, —CO—, —NHCO—, and —O—;
L_{U2} is selected from the group consisting of a single bond, —CH₂—, —NH—, —O—, —CO—, and —CONH—, and

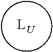

is a single bond or selected from the group consisting of C₁₋₆alkyl, 3-10 membered cycloalkyl, 4-10 membered heterocyclyl, 6-10 membered aryl or 5-10 membered heteroaryl}.

In the present invention, L_{NTM} may be

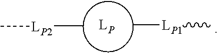

{wherein L_{P1} is selected from the group consisting of a single bond, —O—, —S—, —NH—, —N(C₁₋₄alkyl)-, —CH₂—, —CH(C₁₋₄alkyl)-, —CH₂NH—, and —CH₂CH₂—,
L_{P2} is selected from the group consisting of a single bond, —CO—, —COCH₂—, —NHCO—, —NHCOCH₂—, -HET-, and -HET-CH₂—, wherein HET is a 5-6 membered heterocyclyl or heteroaryl having at least one N, S or O atom, and

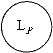

is a single bond, C₁₋₈alkyl substituted with amine group; or a ring selected from the group consisting of 3-10 membered cycloalkyl, 4-10 membered heterocyclyl, 6-10 membered aryl, and 5-10 membered heteroaryl}.

In the present invention,

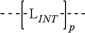

may be

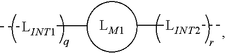

{wherein

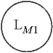

is a single bond; or a ring selected from the group consisting of 3-10 membered cycloalkyl, 4-10 membered heterocyclyl, 6-10 membered aryl, and 5-10 membered heteroaryl,
L_{INT1} and L_{INT2} are each independently selected from the group consisting of —CH₂—, —NH—, —NCH₃—, —O—, —S—, —SO—, —SO₂—, —CO—, —CH₂CH₂O—, —OCH₂CH₂—, —CH₂CH₂S—, —SCH₂CH₂—, —COO—, —CONH—, and —NHCO—, and
q and r are each independently an integer of 1 to 10}.

In an embodiment, the Linker is a Linker contained in the PROTAC compounds provided in Examples 1 to 21 of the present invention.

According to a specific embodiment of the present invention, the compound represented by Formula I is Compounds 1 to 21 provided in Examples 1 to 21 below, a stereoisomer thereof or a pharmaceutically acceptable salt thereof.

In the present invention as used herein, the term “compound” also includes, in addition to single compounds, tautomers, optical isomers (including racemic mixtures), specific enantiomers, or enantiomerically enriched mixtures. The substituent terms used to describe the structure of the compounds of the present invention have the same meanings as commonly used in the field of organic chemistry.

In the present invention, a pharmaceutically acceptable salt refers to any organic acid or inorganic acid addition salt with a concentration having a relatively non-toxic and harmless effective action to patients, wherein side effects caused by this salt do not reduce the beneficial efficacy of the compound represented by Formula I. For example, the pharmaceutically acceptable salt may be an inorganic acid such as hydrochloric acid, phosphoric acid, sulfuric acid, nitric acid, or the like, or an organic acid such as methanesulfonic acid, p-toluenesulfonic acid, formic acid, acetic acid, trifluoroacetic acid, maleic acid, succinic acid, oxalic acid, benzoic acid, tartaric acid, fumaric acid, manderic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, or hydroiodic acid, but is not limited thereto.

### Method for Preparing NLRP3 Protein Degradation Inducing Compound

The compound represented by Formula I of the present invention, a stereoisomer thereof, or a pharmaceutically acceptable salt thereof may be synthesized through reactions such as the following Reaction Schemes 1 to 3 by synthetic methods known in the technical field of organic chemistry or modification and derivatization techniques obvious to those skilled in the art:

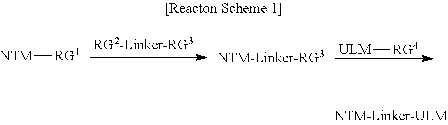

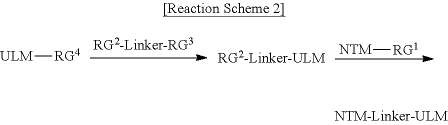

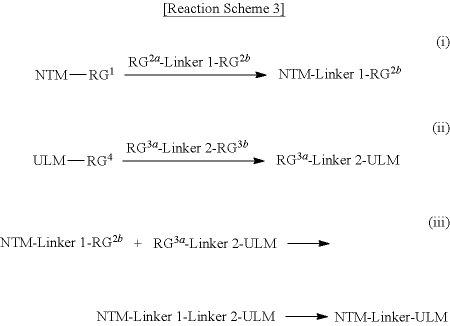

In Reaction Schemes 1 to 3 above, NTM, Linker, and ULM are the above-defined groups or reaction derivatives thereof, and RG¹, RG², RG^{2a}, RG^{2b}, RG³, RG^{3a}, RG^{3b}, and RG⁴ are moieties containing suitable reactive groups capable of linking together with the PROTAC compound intermediate represented by Formula I through covalent bond formation in the field of organic synthesis. The covalent bond formation may be performed through synthesis reactions such as amide formation, ester formation, carbamate formation, urea formation, ether formation, amine formation, and single bonds, double bonds formation between various carbons, click chemistry, and the like, depending on the specific reactive group, but not limited to.

Variations of each step in the Reaction Scheme above may include one or multiple synthetic steps. Isolation and purification of the product may be achieved by standard procedures known to those skilled in the art of organic chemistry.

In the process of preparing the compound represented by Formula I of the present invention, the compounds in the form of intermediates shown in Reaction Schemes 1 to 3 above are also included in the scope of the present invention.

Specifically, the present invention provides variants of NTM moieties in the form of NTM-RG¹, NTM-Linker-RG³ or NTM-Linker 1-RG^{2b}, and provides variants of ULM moieties in the form of ULM-RG⁴, RG^{3a}-Linker 2-ULM or RG²-Linker-ULM.

### Use of NLRP3 Protein Degradation Inducing Compound

In one aspect, the present invention provides a composition for degrading NLRP3 protein comprising a compound represented by Formula I, a stereoisomer thereof, or a pharmaceutically acceptable salt thereof.

As shown in FIG. 1, the compound represented by Formula I according to the present invention may recruit the NLRP3 protein, which is a target protein, to the E3 ubiquitin ligase and induce ubiquitination of the NLRP3 protein, thereby inducing degradation of NLRP3 protein by the ubiquitin-proteasome system (UPS) in cells. As a result of treating immune cells with the compounds according to Examples of the present invention, which represent the compound of Formula I, it was confirmed that the activity of IL-1β mediated by the NRLP3 protein that is not degraded was inhibited (Experimental Example 1), which indicates that the bifunctional compound according to the technical idea of the present invention, as PROTAC, effectively degrades the target protein, NLRP3 protein. Therefore, the compound represented by Formula I of the present invention may be usefully employed to induce degradation of NLRP3 protein.

In an embodiment, the composition for degrading NLRP3 protein may be administered to mammals, including humans, to degrade NLRP3 protein. In this case, the composition may be a pharmaceutical composition further comprising at least one type of pharmaceutically acceptable carrier, and may be specifically used for preventing or treating NLRP3 inflammasome-related diseases.

The therapeutic efficacy of the compound represented by Formula I of the present invention for NLRP3 inflammasome-related diseases may be judged from the level of inhibition of the activity of IL-1β, a potent inflammatory cytokine released by activation of NRLP3 inflammasome, which may be specifically confirmed through the method for measuring IL-1β activity presented in Experimental Example 2 of the present invention. In the present invention, the THP-1 cell line was treated with the compounds of Examples representing the compound of Formula I, and as a result, it was confirmed that IL-1β activity, which is an indicator of NLRP3 inflammasome-related diseases, was reduced. Accordingly, the compound represented by Formula I of the present invention may induce degradation of NLRP3 protein, thereby making it possible to be usefully employed for preventing or treating NLRP3 inflammasome-related diseases.

In the present invention, the term “NLRP3 inflammasome-related disease” refers to a disease capable of being mediated by an inflammatory pathway activated by the NLRP3 inflammasome, which means a disease or condition capable of being treated, alleviated, delayed, inhibited, or prevented by degradation of NLRP3 protein. The specific names and scope of NLRP3 inflammasome-related diseases are known in the art (document [Swanson et al. Nature Reviews Immunology 19.8 (2019): 477-489.], and the like).

For example, NLRP3 inflammasome-related diseases may include central nervous system diseases (such as Alzheimer's disease, multiple sclerosis, amyotrophic sclerosis or Parkinson's disease), metabolic disorders (such as type 1 diabetes, type 2 diabetes, hypertension, atherosclerosis, obesity or gout), cardiovascular diseases (such as myocardial infarction or giant cell arteritis), respiratory diseases (such as asthma, COPD, silicosis, pulmonary diuretic fibrosis, or allergic airway inflammation), liver diseases (such as non-alcoholic steatohepatitis [NASH], viral hepatitis, or cirrhosis), pancreatic diseases (such as acute pancreatitis or chronic pancreatitis), kidney diseases (such as nephropathy, acute kidney injury, or chronic kidney injury), bowel diseases (including inflammatory bowel disease such as Crohn's disease or ulcerative colitis), skin diseases (such as psoriasis), musculoskeletal diseases (such as scleroderma), bone diseases (such as osteoarthritis, osteoporosis, or osteopetrosis), ocular diseases (such as glaucoma or macular degeneration), inflammation after viral infection (such as inflammation after infection with HIV, influenza, chikungunya, or COVID-19 virus), autoimmune diseases (such as rheumatoid arthritis, systemic lupus erythematosus, graft-versus-host disease [GVHD], autoimmune thyroiditis, or autoimmune encephalitis), cancer or tumor (such as myelodysplastic syndrome, non-small cell lung cancer, metastatic lung cancer, gastric cancer, acute lymphocytic leukemia [ALL], acute myelogenous leukemia [AML], chronic myelogenous leukemia [CML], promyelocytic leukemia, Langerhans' cell histiocytosis, or multiple myeloma), and other inflammatory diseases (such as contact hypersensitivity, traumatic brain injury, or Cryopyrin-associated periodic syndromes [CAPS]), and the like, but not limited to.

The pharmaceutical composition of the present invention may be a combination composition further comprising at least one type of drug of the same or similar class capable of helping treat, alleviate, delay, inhibit, or prevent NLRP3 inflammasome-related diseases.

The pharmaceutical composition of the present invention may be formulated through conventional methods in the field of pharmaceuticals, and may be formulated in various forms depending on specific types of NLRP3 inflammasome-related diseases and drug components in combination therewith.

The pharmaceutical composition of the present disclosure may be administered orally or parenterally (for example, intravenous, subcutaneous, intraperitoneal, or topical application) according to the desired method, and the dosage varies depending on the patient's weight, age, sex, health condition, diet, administration time, administration method, excretion rate, and severity of the disease, and the like.

In still another embodiment, the composition for degrading NLRP3 protein may be treated with a sample in vitro to degrade NLRP3 protein in the sample. The sample may be cells, cell cultures, body fluids or tissues of mammals including humans, and may be used for diagnostic or therapeutic purposes.

### Advantageous Effects

The compound of the present invention exhibits an effect of inducing degradation of NLRP3 protein in cells, and thus may be usefully employed for preventing or treating NLRP3 inflammasome-related diseases.

### DESCRIPTION OF DRAWINGS

FIG. 1 shows the principle that the bifunctional compound (Proteolysis Targeting Chimeras; PROTAC) according to the present invention recruits the target protein, NLRP3, to an E3 ubiquitin ligase and then induces ubiquitination, thus leading to degradation;

FIG. 2 shows a binding structure of an NLRP3 protein binding moiety (NTM) binding to the Walker site in the NACHT domain of the NLRP3 protein;

FIG. 3 shows a binding structure of a CRBN E3 ubiquitin ligase binding moiety (ULM); and

FIG. 4 shows a binding structure of a VHL E3 ubiquitin ligase binding moiety (ULM).

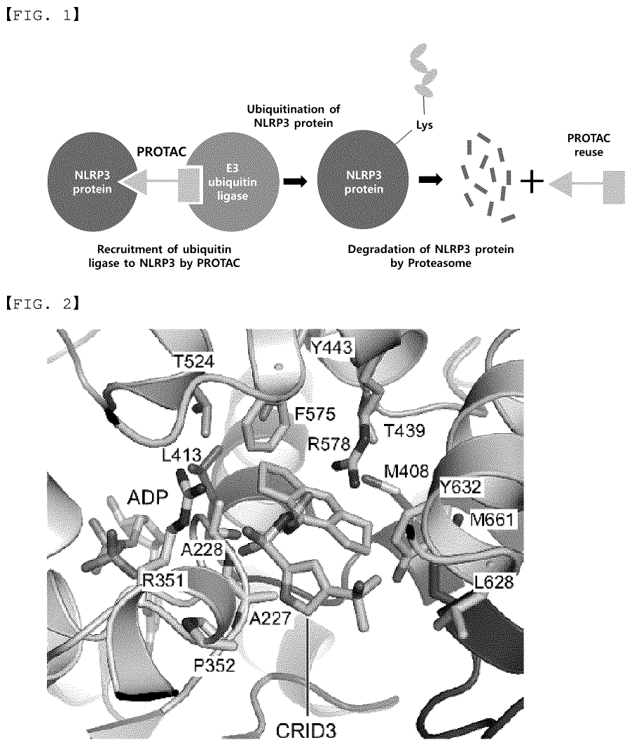

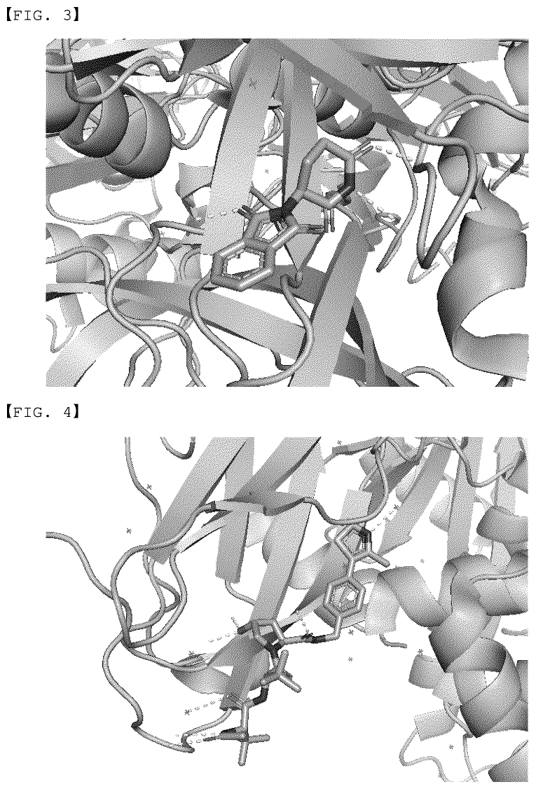

### BEST MODE

Hereinafter, the constitution and effects of the present disclosure will be described in more detail through Examples and Experimental Examples. These Examples and Experimental Examples are only provided for illustrating the present disclosure, but the scope of the present disclosure is not limited by these Examples. All documents cited throughout the present application are hereby expressly incorporated herein by reference in their entirety.

As the best mode for carrying out the present invention, provided are synthetic methods for compounds 1 to 21 listed in Table below.

TABLE 1

| Compound | Structure |
|---|---|
| 1 | 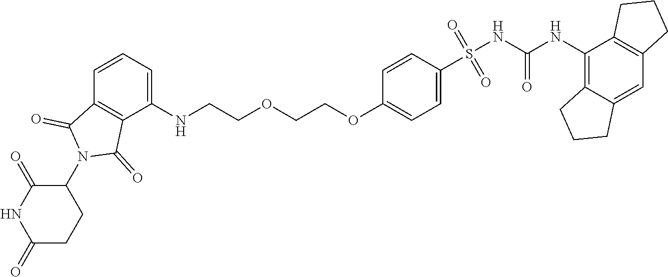 [SMILES: O=C1CCC(N2C(=O)c3cccc(NCCOCCOc4ccc(S(=O)(=O)NC(=O)Nc5c6c(cc7c5CCC7)CCC6)cc4)c3C2=O)C(=O)N1] |
| 2 | 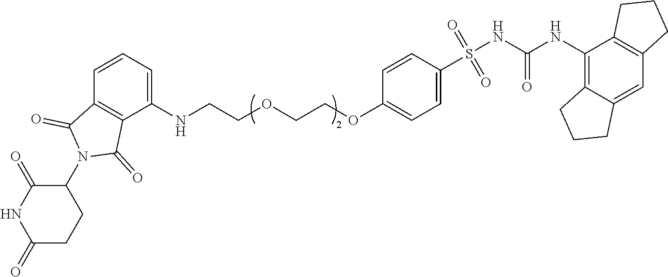 |
| 3 | 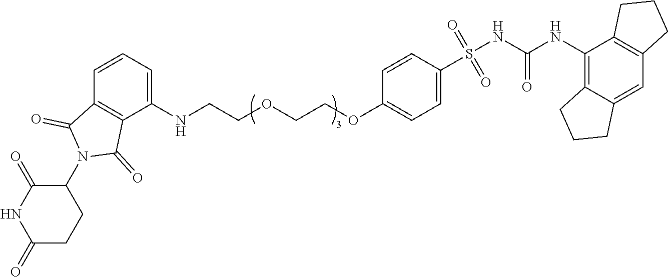 |
| 4 | 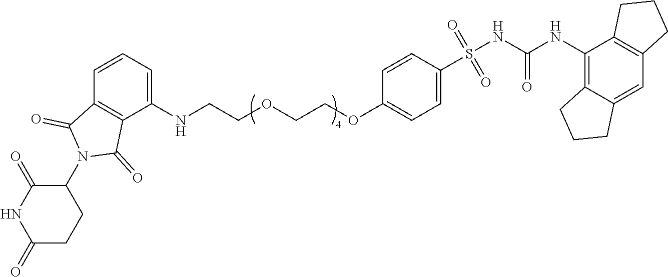 |
| 5 | 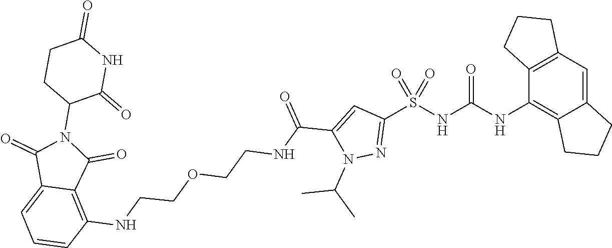 [SMILES: CC(C)n1nc(S(=O)(=O)NC(=O)Nc2c3c(cc4c2CCC4)CCC3)cc1C(=O)NCCOCCNc1cccc2c1C(=O)N(C1CCC(=O)NC1=O)C2=O] |
| 6 | 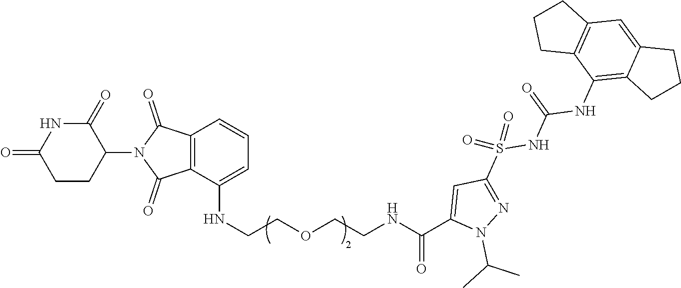 |
| 7 | 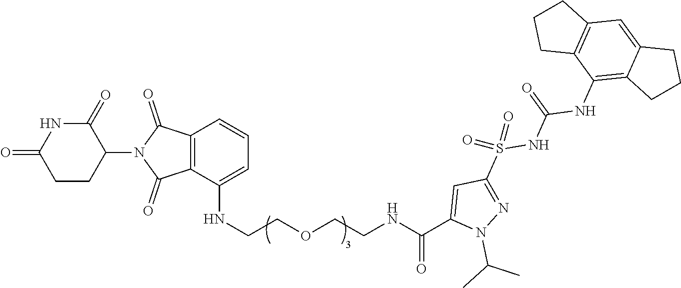 |
| 8 | 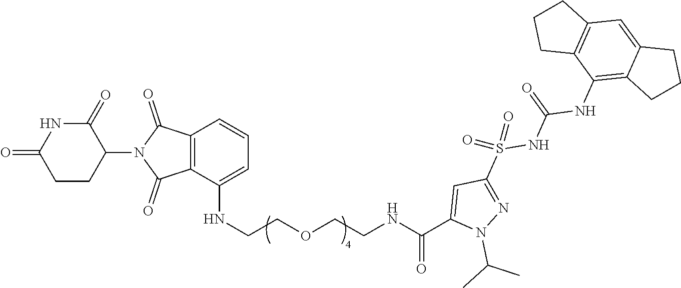 |
| 9 | 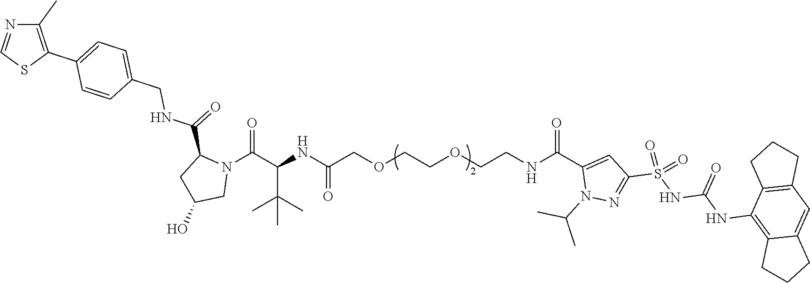 |
| 10 | 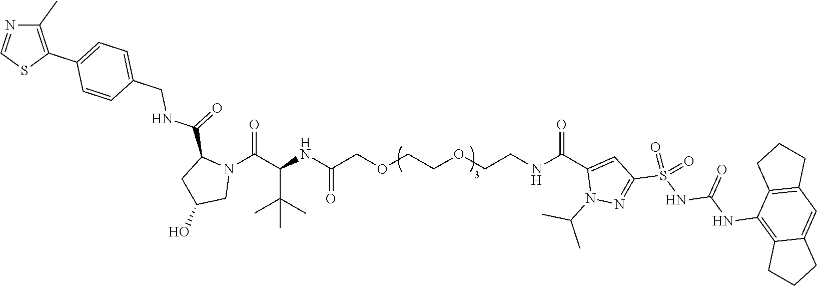 |
| 11 | 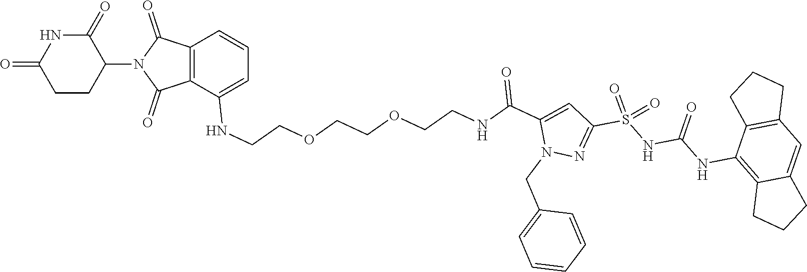 [SMILES: O=C1CCC(N2C(=O)c3cccc(NCCOCCOCCNC(=O)c4cc(S(=O)(=O)NC(=O)Nc5c6c(cc7c5CCC7)CCC6)nn4Cc4ccccc4)c3C2=O)C(=O)N1] |
| 12 | 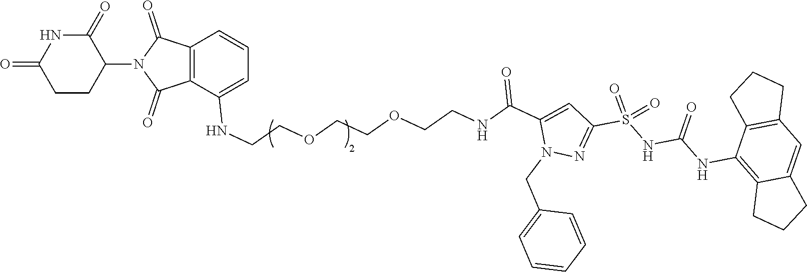 |
| 13 | 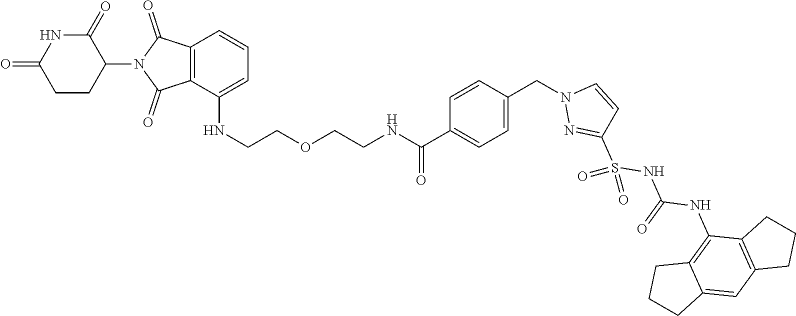 [SMILES: O=C1CCC(N2C(=O)c3cccc(NCCOCCNC(=O)c4ccc(Cn5ccc(S(=O)(=O)NC(=O)Nc6c7c(cc8c6CCC8)CCC7)n5)cc4)c3C2=O)C(=O)N1] |
| 14 | 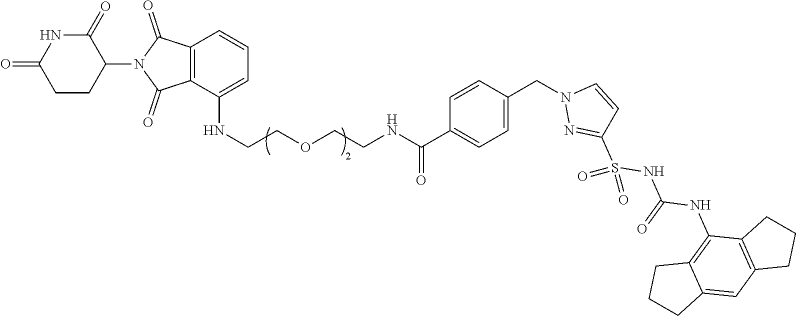 |
| 15 | 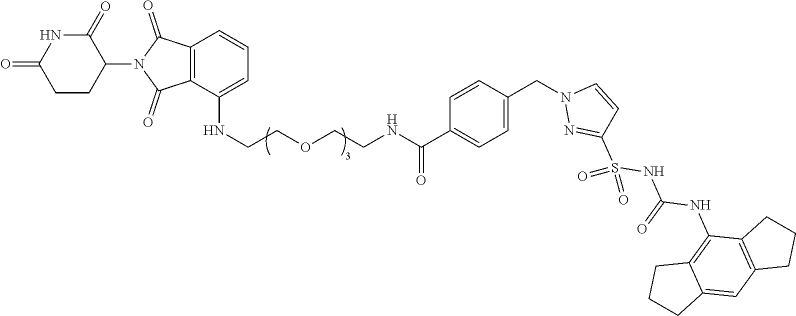 |
| 16 | 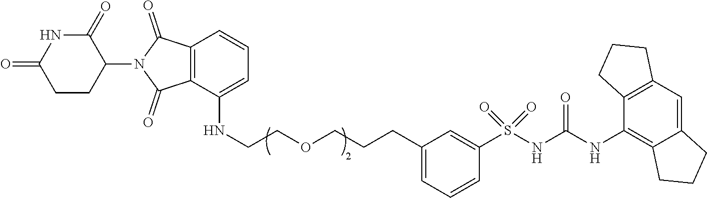 |
| 17 | 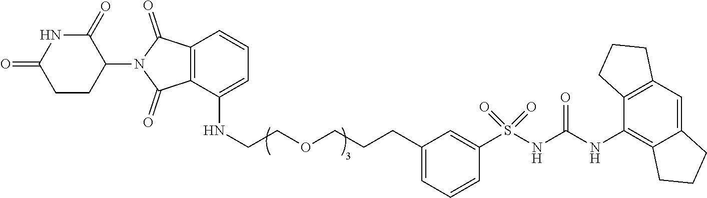 |
| 18 | 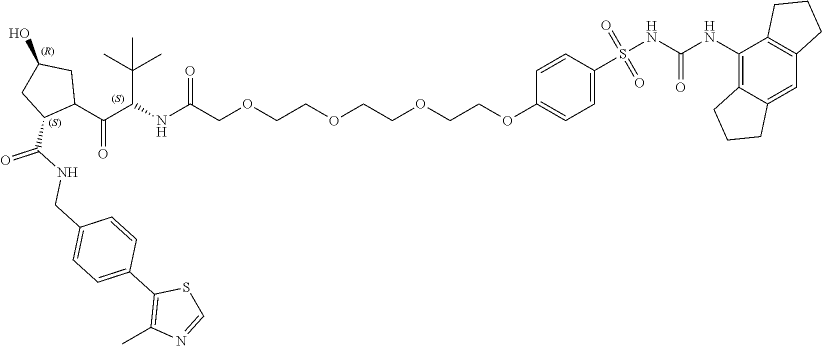 |
| 19 | 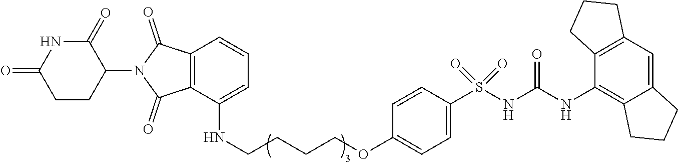 |
| 20 | 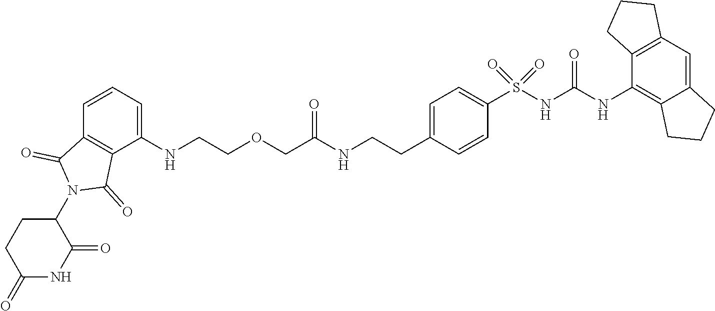 [SMILES: O=C(COCCNc1cccc2c1C(=O)N(C1CCC(=O)NC1=O)C2=O)NCCc1ccc(S(=O)(=O)NC(=O)Nc2c3c(cc4c2CCC4)CCC3)cc1] |
| 21 | 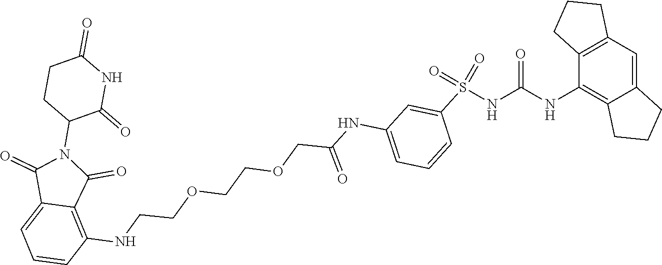 [SMILES: O=C1CCC(N2C(=O)c3cccc(NCCOCCOCC(=O)Nc4cccc(S(=O)(=O)NC(=O)Nc5c6c(cc7c5CCC7)CCC6)c4)c3C2=O)C(=O)N1] |

The names of the compounds 1 to 21 are as follows.
4-(2-(2-((2-(2,6-dioxopiperidin-3-yl)-1,3-dioxoisoindolin-4-yl)amino)ethoxy)ethoxy)-N-((1,2,3,5,6,7-hexahydro-s-indacen-4-yl)carbamoyl)benzenesulfonamide (Compound 1);
4-(2-(2-(2-((2-(2,6-dioxopiperidin-3-yl)-1,3-dioxoisoindolin-4-yl)amino)ethoxy)ethoxy)ethoxy)-N-((1,2,3,5,6,7-hexahydro-s-indacen-4-yl)carbamoyl)benzenesulfonamide (Compound 2);
4-(2-(2-(2-(2-((2-(2,6-dioxopiperidin-3-yl)-1,3-dioxoisoindolin-4-yl)amino)ethoxy)ethoxy)ethoxy)ethoxy)-N-((1,2,3,5,6,7-hexahydro-s-indacen-4-yl)carbamoyl)benzenesulfonamide (Compound 3);
4-((14-((2-(2,6-dioxopiperidin-3-yl)-1,3-dioxoisoindolin-4-yl)amino)-3,6,9,12-tetraoxatetradecyl)oxy)-N-((1,2,3,5,6,7-hexahydro-s-indacen-4-yl)carbamoyl)benzenesulfonamide (Compound 4);
N-(2-(2-((2-(2,6-dioxopiperidin-3-yl)-1,3-dioxoisoindolin-4-yl)amino)ethoxy)ethyl)-3-(N-((1,2,3,5,6,7-hexahydro-s-indacen-4-yl)carbamoyl)sulfamoyl)-1-isopropyl-1H-pyrazol-5-carboxamide (Compound 5);
N-(2-(2-(2-((2-(2,6-dioxopiperidin-3-yl)-1,3-dioxoisoindolin-4-yl)amino)ethoxy)ethoxy)ethyl)-3-(N-((1,2,3,5,6,7-hexahydro-s-indacen-4-yl)carbamoyl)sulfamoyl)-1-isopropyl-1H-pyrazol-5-carboxamide (Compound 6);
N-(2-(2-(2-(2-((2-(2,6-dioxopiperidin-3-yl)-1,3-dioxoisoindolin-4-yl)amino)ethoxy)ethoxy)ethoxy)ethyl)-3-(N-((1,2,3,5,6,7-hexahydro-s-indacen-4-yl)carbamoyl)sulfamoyl)-1-isopropyl-1H-pyrazol-5-carboxamide (Compound 7);
N-(14((2-(2,6-dioxopiperidin-3-yl)-1,3-dioxoisoindolin-4-yl)amino)-3,6,9,23-tetraoxatetradecyl)-3-(N-((1,2,3,5,6,7-hexahydro-s-indacen-4-yl)carbamoyl)sulfamoyl)-1-isopropyl-1H-pyrazol-5-carboxamide (Compound 8);
3-(N-((1,2,3,5,6,7-hexahydro-s-indacen-4-yl)carbamoyl)sulfamoyl)-N—((S)-13-((2S,4R)-4-hydroxy-2-((4-(4-methylthiazol-5-yl)benzyl)carbamoyl)pyrrolidine-1-carbonyl)-14,14-dimethyl-11-oxo-3,6,9-trioxa-12-azapentadecyl-1-isopropyl-1H-pyrazol-5-carboxamide (Compound 9);
3-(N-((1,2,3,5,6,7-hexahydro-s-indacen-4-yl)carbamoyl)sulfamoyl)-N—((S)-16-((2S,4R)-4-hydroxy-2-((4-(4-methylthiazol-5-yl)benzyl)carbamoyl)pyrrolidine-1-carbonyl)17,17-dimethyl-14-oxo-3,6,9,12-tetraoxa-15-azaoctadecyl)-1-isopropyl-1H-pyrazol-5-carboxamide (Compound 10);
1-benzyl-N-(2-(2-(2-((2-(2,6-dioxopiperidin-3-yl)-1,3-dioxoisoindolin-4-yl)amino)ethoxy)ethoxy)ethyl)-3-(N-((1,2,3,5,6,7-hexahydro-s-indacen-4-yl)carbamoyl)sulfamoyl)-1H-pyrazol-5-carboxamide (Compound 11);
1-benzyl-N-(2-(2-(2-(2-((2-(2,6-dioxoisoindolin-4-yl)amino)ethoxy)ethoxy)ethoxy)ethyl)-3-(N-((1,2,3,5,6,7-hexahydro-s-indacen-4-yl)carbamoyl)sulfamoyl)-1H-pyrazol-5-carboxamide (Compound 12);
N-(2-(2-((2-(2,6-dioxopiperidin-3-yl)-1,3-dioxoisoindolin-4-yl)amino)ethoxy)ethyl)-4-((3-(N-((1,2,3,5,6,7-hexahydro-s-indacen-4-yl)carbamoyl)sulfamoyl)-1H-pyrazol-1-yl)methyl)benzamide (Compound 13);
N-(2-(2-(2-((2-(2,6-dioxopiperidin-3-yl)-1,3-dioxoisoindolin-4-yl)amino)ethoxy)ethoxy)ethyl-4-((3-(N-((1,2,3,5,6,7-hexahydro-s-indacen-4-yl)carbamoyl)sulfamoyl)-1H-pyrazol-1-yl)methyl)benzamide (Compound 14);
N-(2-(2-(2-(2-((2-(2,6-dioxopiperidin-3-yl)-1,3-dioxoisoindolin-4-yl)amino)ethoxy)ethoxy)ethoxy)ethyl-4-((3-(N-((1,2,3,5,6,7-hexahydro-s-indacen-4-yl)carbamoyl)sulfamoyl)-1H-pyrazol-1-yl)methyl)benzamide (Compound 15);
3-(2-(2-(2-((2-(2,6-dioxopiperidin-3-yl)-1,3-dioxoisoindolin-4-yl)amino)ethoxy)ethoxyethoxy)-N-((1,2,3,5,6,7-hexahydro-s-indacen-4-yl)carbamoyl)benzenesulfonamide (Compound 16);
3-(2-(2-(2-(2-((2-(2,6-dioxopiperidin-3-yl)-1,3-dioxoisoindolin-4-yl)amino)ethoxy)ethoxy)ethoxy)ethoxy)-N-((1,2,3,5,6,7-hexahydro-s-indacen-4-yl)carbamoyl)benzenesulfonamide (Compound 17);
(2S,4R)-1-((S)-2-(tert-butyl)-14-(4-(N-((1,2,3,5,6,7-hexahydro-s-indacen-4)-yl)carbamoyl)sulfamoyl)phenoxy)-4-oxo-6,8,12-trioxa-3-azatetradecanoyl)-4-hydroxy-N-(4-(4-methylthiazol-5-yl)benzyl)pyrrolidine-2-carboxamide (Compound 18);
4-((10-((2-(2,6-dioxopiperidin-3-yl)-1,3-dioxoisoindolin-4-yl)amino)decyl)oxy)-N-((1,2,3,5,6,7-hexahydro-s-indacen-4-yl)carbamoyl)benzenesulfonamide (Compound 19);
2-(2-((2-(2,6-dioxopiperidin-3-yl)-1,3-dioxoisoindolin-4-yl)amino)ethoxy)-N-(4 (N-((1,2,3,5,6,7-hexahydro-s-indacen-4-yl)carbamoyl)sulfamoyl)phenethyl)acetamide (Compound 20); and
2-(2-(2-((2-(2,6-dioxopiperidin-3-yl)-1,3-dioxoisoindolin-4-yl)amino)ethoxy)ethoxy)-N-(3-(N-(1,2,3,5,6,7-hexahydro-s-indacen-4-yl)carbamoyl)sulfamoyl)phenyl)acetamide (Compound 21).

The compounds of the present invention were purified and structurally analyzed according to the method below.

### Analytical Instruments

LCMS: Agilent Single Quad system (1260)
NMR: BRUKER Advance Nanobay 400 MHz, Bruker AV-600 600 Mhz
HPLC: Agilent 1260 II LC

### LCMS Analysis Method

LCMS data were recorded by an Agilent Single Quad system (1260) equipped with an electron spray ionization device. 0.0375% TFA in water (solvent A) and 0.01875% TFA in acetonitrile (solvent B) were used as mobile phases. As a column, Poroshell 120 EC-C18 (2.1*50) mm, 2.7 μm was used.

### HPLC Analysis Method

As HPLC, Agilent 1260 II LC was used, and 0.0375% TFA in water (solvent A) and 0.01875% TFA in acetonitrile (solvent B) were used as mobile phases. As a column, Zobrax Eclipse Plus C18 (4.6*150) mm, 3.5 μm was used.

### NMR Analysis Method

¹H NMR spectra were recorded using a BRUKER Advance Nanobay 400 MHz/5 mm Probe (BBO) and a Bruker AV-600 600 MHz.

### <Example 1> Synthesis of 4-(2-(2-((2-(2,6-dioxopiperidin-3-yl)-1,3-dioxoisoindolin-4-yl)amino)ethoxy)ethoxy)-N-((1,2,3,5,6,7-hexahydro-s-indacen-4-yl)carbamoyl)benzenesulfonamide (Compound 1)

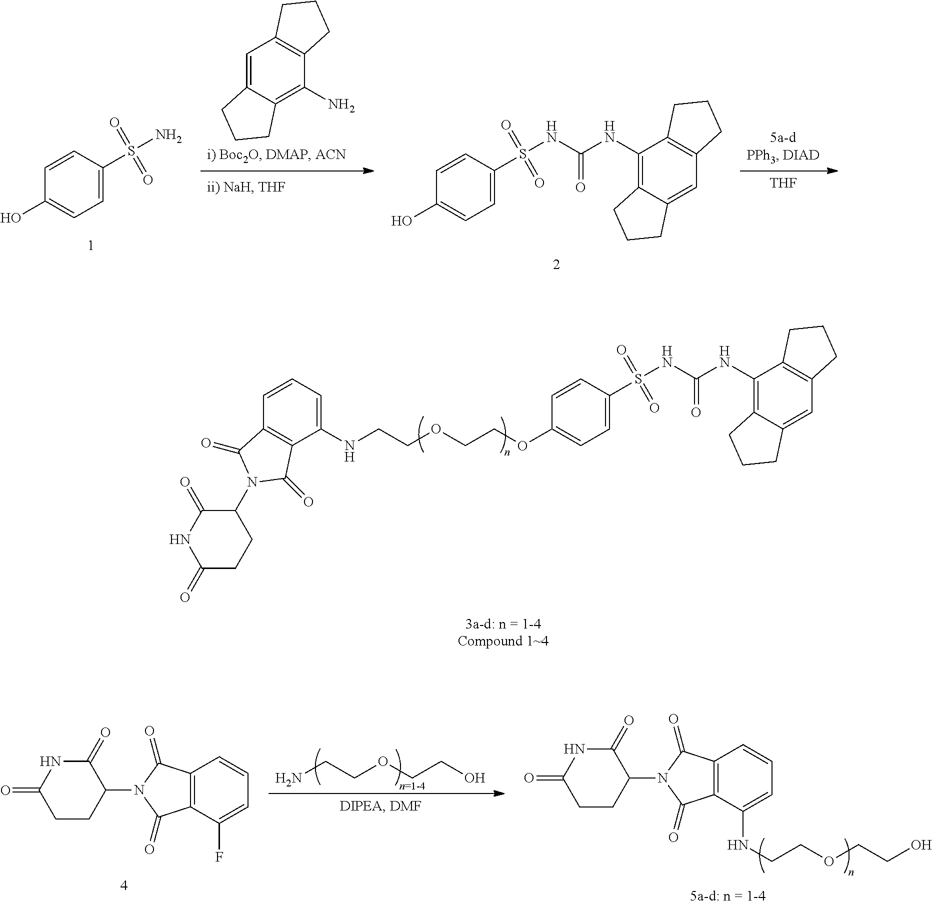

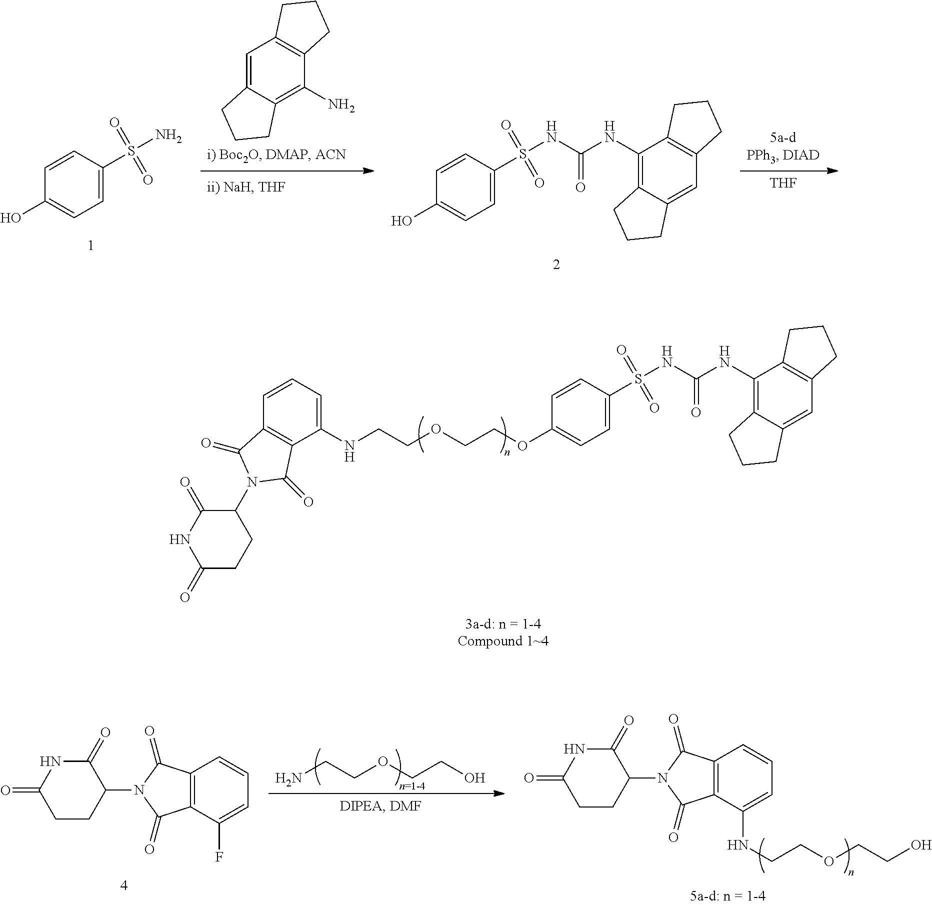

### Step 1. Synthesis of N-((1,2,3,5,6,7-hexahydro-s-indacen-4-yl)carbamoyl)-4-hydroxybenzenesulfonamide (2)

To a solution of di-t-butyldicarbonate (10.6 g, 48.48 mmol) in anhydrous ACN (60 mL) was added DMAP (2.12 g, 17.32 mmol), and stirred at room temperature for 5 min. 1,2,3,5,6,7-hexahydro-s-indacen-4-amine (6.00 g, 34.63 mmol) was dissolved in ACN and added to a resulting reaction mixture. The other flask was prepared for sulfonamide intermediate. To 4-hydroxybenzenesulfonamide (6.00 g, 34.63 mmol) in anhydrous THF (60 mL) was added NaH (1.39 g, 34.63 mmol, 60% dispersion in mineral oil) at 0° C. and stirred at room temperature for 30 min under nitrogen atmosphere. Again cooled to 0° C., a mixture of 1,2,3,5,6,7-hexahydro-s-indacen-4-amine was added and stirred at room temperature for 16 h. The main peak of the desired mass was confirmed by LCMS. The mixture was filtered and washed with ACN (30 mL). Water was added and acidified to pH 5 with 1N HCl aqueous solution. After stirring the mixture until a suspension occurred, the solid was filtered under reduced pressure and dried for 5 h. To the solid was added EtOAc (30 mL) for solidification and filtered. The filter cake was dried for 18 h to obtain the title compound as a white solid (6.92 g, 18.58 mmol, 54% yield).

MS(M+H)⁺=373.1

### Step 2. Synthesis of 4-(2-(2-((2-(2,6-dioxopiperidin-3-yl)-1,3-dioxoisoindolin-4-yl)amino)ethoxy)ethoxy)-N-((1,2,3,5,6,7-hexahydro-s-indacen-4-yl)carbamoyl)benzenesulfonamide (3a) (Compound 1)

To a solution of N-((1,2,3,5,6,7-hexahydro-s-indacen-4-yl)carbamoyl)-4-hydroxybenzenesulfonamide (150 mg, 0.40 mmol) in THF (3 mL) was added 2-(2,6-dioxopiperidin-3-yl)-4-((2-(2-hydroxyethoxy)ethyl)amino)isoindoline-1,3-dione (157 mg, 0.43 mmol), Triphenylphosphine (158 mg, 0.60 mmol) and DIAD (0.12 mL, 0.60 mmol) and the mixture was stirred at room temperature for 20 h. The main peak of the desired mass was confirmed by LCMS. EtOAc (30 mL) and water (30 mL) were added and layers were separated. The aqueous phase was extracted with EtOAc (10 mL×2). The combined organic phase was washed with brine (30 mL), dried over MgSO₄, filtered and concentrated under reduced pressure. The residue was purified by Medium pressure liquid chromatography (MC:EA=3:1→MC:MeOH=97:3) and Preparative-High Pressure Liquid Chromatography to obtain the title compound as a yellow solid (14 mg, 4.8%).

¹H NMR (600 MHz, (CD₃)₂SO) δ=11.10 (s, 1H), 10.35 (s, 1H), 8.90 (s, 1H), 7.79 (d, J=8.4 Hz, 2H), 7.55 (t, J=7.8 Hz, 1H), 7.04 (t, J=4.5 Hz, 2H), 6.96 (s, 1H), 6.89 (d, J=8.4 Hz, 2H), 6.53 (t, J=5.7 Hz, 1H), 5.07 (s, 1H), 4.23 (s, 1H), 3.53 (q, J=4.8 Hz, 2H), 3.40 (t, J=5.4 Hz, 3H), 3.30 (q, J=5.6 Hz, 2H), 2.92-2.86 (m, 1H), 2.76 (t, J=4.2 Hz, 3H), 2.61-2.56 (m, 1H), 2.54 (d, J=7.2 Hz, 4H), 2.51 (t, J=1.5 Hz, 2H), 2.04-2.00 (m, 1H), 1.93-1.88 (m, 4H).

MS(M+H)⁺=716.2

### <Example 2> Synthesis of 4-(2-(2-(2-((2-(2,6-dioxopiperidin-3-yl)-1,3-dioxoisoindolin-4-yl)amino)ethoxy)ethoxy)ethoxy)-N-((1,2,3,5,6,7-hexahydro-s-indacen-4-yl)carbamoyl)benzenesulfonamide (Compound 2)

### Step 3. Synthesis of 4-(2-(2-(2-((2-(2,6-dioxopiperidin-3-yl)-1,3-dioxoisoindolin-4-yl)amino)ethoxy)ethoxy)ethoxy)-N-((1,2,3,5,6,7-hexahydro-s-indacen-4-yl)carbamoyl)benzenesulfonamide (3b) (Compound 2)

To a solution of N-((1,2,3,5,6,7-hexahydro-s-indacen-4-yl)carbamoyl)-4-hydroxybenzenesulfonamide (120 mg, 0.323 mmol) in THF (3 mL) was added 2-(2,6-dioxopiperidin-3-yl)-4-((2-(2-(2-hydroxyethoxy)ethoxy)ethyl)amino)isoindoline-1,3-dione (131 mg, 0.323 mmol), Triphenylphosphine (127 mg, 0.485 mmol) and DIAD (95.4 μL, 0.485 mmol) and the mixture was stirred at room temperature for 20 h. The main peak of the desired mass was confirmed by LCMS. EtOAc (30 mL) and water (30 mL) were added and layers were separated. The aqueous phase was extracted with EtOAc (10 mL×2). The combined organic phase was washed with brine (30 mL), dried over MgSO₄, filtered and concentrated under reduced pressure. The residue was purified by silica gel chromatography (Hex:EA=1:3→MC:MeOH=97:3) to obtain the title compound as a yellow solid (18 mg, 7%).

¹H NMR (600 MHz, (CD₃)₂SO) δ=11.09 (br s, 1H), 7.63-7.54 (m, 3H), 7.40 (t, J=5.4 Hz, 1H), 7.14 (d, J=8.4 Hz, 1H), 7.03 (d, J=7.2 Hz, 1H), 6.88 (m, 2H), 6.59 (t, J=5.7 Hz, 1H), 6.33 (s, 1H), 5.05 (q, J=6.2 Hz, 1H), 4.52 (s, 1H), 3.59 (t, J=5.7 Hz, 2H), 3.53-3.49 (m, 2H), 3.47-3.42 (m, 4H), 3.38-3.34 (m, 4H), 3.36 (d, J=6.0 Hz, 2H), 2.91-2.81 (m, 3H), 2.71 (q, J=7.5 Hz, 3H), 2.61-2.52 (m, 5H), 2.04-2.00 (m, 1H), 1.98-1.90 (m, 4H).

MS(M+H)⁺=760.3

### <Example 3> Synthesis of 4-(2-(2-(2-(2-((2-(2,6-dioxopiperidin-3-yl)-1,3-dioxoisoindolin-4-yl)amino)ethoxy)ethoxy)ethoxy)ethoxy)-N-((1,2,3,5,6,7-hexahydro-s-indacen-4-yl)carbamoyl)benzenesulfonamide (Compound 3)

### Step 4. Synthesis of 4-(2-(2-(2-(2-((2-(2,6-dioxopiperidin-3-yl)-1,3-dioxoisoindolin-4-yl)amino)ethoxy)ethoxy)ethoxy)ethoxy)-N-((1,2,3,5,6,7-hexahydro-s-indacen-4-yl)carbamoyl)benzenesulfonamide (3c) (Compound 3)

The title compound (18 mg, 5% yield) was obtained as a yellow solid in a similar manner to the synthesis scheme of Compound 3a of Example 1.

¹H NMR (600 MHz, (CD₃)₂SO) δ=11.89 (br s, 1H), 7.60 (q, J=3.4 Hz, 2H), 7.58 (d, J=8.4 Hz, 1H), 7.40 (t, J=5.4 Hz, 1H), 7.15 (d, J=8.4 Hz, 1H), 7.05 (d, J=7.2 Hz, 1H), 6.89 (d, J=8.4 Hz, 2H), 6.60 (t, J=5.7 Hz, 1H), 6.34 (s, 1H), 5.06 (q, J=6.2 Hz, 1H), 4.52 (s, 1H), 3.62 (t, J=5.7 Hz, 2H), 3.56 (q, J=3.2 Hz, 2H), 3.51 (q, J=3.0 Hz, 2H), 3.47-3.45 (m, 4H), 3.42-3.39 (m, 4H), 3.36 (d, J=6.0 Hz, 2H), 2.91-2.85 (m, 1H), 2.83 (q, J=5.8 Hz, 2H), 2.71 (t, J=7.5 Hz, 4H), 2.58 (t, J=7.2 Hz, 4H), 2.51 (t, J=7.5 Hz, 2H), 2.04-2.00 (m, 1H), 1.98-1.93 (m, 4H).

MS(M+H)⁺=804.3

### <Example 4> Synthesis of 4-((14-((2-(2,6-dioxopiperidin-3-yl)-1,3-dioxoisoindolin-4-yl)amino)-3,6,9,12-tetraoxatetradecyl)oxy)-N-((1,2,3,5,6,7-hexahydro-s-indacen-4-yl)carbamoyl)benzenesulfonamide (Compound 4)

### Step 5. Synthesis of 4-((14-((2-(2,6-dioxopiperidin-3-yl)-1,3-dioxoisoindolin-4-yl)amino)-3,6,9,12-tetraoxatetradecyl)oxy)-N-((1,2,3,5,6,7-hexahydro-s-indacen-4-yl)carbamoyl)benzenesulfonamide (3d) (Compound 4)

The title compound (50 mg, 14% yield) was obtained as a yellow solid in a similar manner to the synthesis scheme of Compound 3a of Example 1.

¹H NMR (600 MHz, (CD₃)₂SO) δ=11.10 (s, 1H), 10.36 (s, 1H), 7.62-7.60 (m, 2H), 7.58 (t, J=4.2 Hz, 1H), 7.40 (t, J=6.0 Hz, 1H), 7.15 (d, J=8.4 Hz, 1H), 7.05 (d, J=7.2 Hz, 1H), 6.91-6.88 (m, 2H), 6.61 (t, J=5.7 Hz, 1H), 6.34 (s, 1H), 5.06 (q, J=6.2 Hz, 1H), 4.53 (s, 1H), 3.62 (t, J=5.4 Hz, 2H), 3.57-3.56 (m, 2H), 3.53-3.52 (m, 2H), 3.51-3.49 (m, 2H), 3.49-3.46 (m, 4H), 3.46-3.44 (m, 2H), 3.36 (t, J=6.0 Hz, 4H), 2.80 (q, J=6.9 Hz, 2H), 2.71 (t, J=7.5 Hz, 4H), 2.58 (t, J=7.2 Hz, 4H), 2.04-2.01 (m, 1H), 1.98-1.93 (m, 4H)

MS(M+H)⁺=848.3

### Step 6. Synthesis of 2-(2,6-dioxopiperidin-3-yl)-4-((2-(2-hydroxyethoxy)ethyl)amino)isoindoline-1,3-dione (5a)

To a solution of 2-(2,6-dioxo-3-piperidyl)-4-fluoro-isoindoline-1,3-dione (0.30 g, 1.09 mmol) and 2-(2-aminoethoxy)ethan-1-ol (540 mg, 3.60 mmol) in DMF (3 mL) was added DIPEA (0.31 mL, 2.17 mmol) and the resulting mixture was stirred at 80° C. for 16 h. The reaction was confirmed to completion by LCMS. The reaction mixture was cooled to room temperature, EtOAc (30 mL) and water (30 mL) were added and layers were separated. The organic phase was washed with water (10 mL×2) and brine (100 mL), dried over Na₂SO₄, filtered and the filtrate was concentrated under reduced pressure. The residue was purified by silica gel chromatography (Dichloromethane:Methanol=9:1) to obtain the title compound as a yellow oil (157 mg, 0.434 mmol, 40% yield).

MS(M+H)⁺=362.1

### Step 7. Synthesis of 2-(2,6-dioxopiperidin-3-yl)-4-((2-(2-(2-hydroxyethoxy)ethoxy)ethyl)amino)isoindoline-1,3-dione (5b)

To a solution of 2-(2,6-dioxo-3-piperidyl)-4-fluoro-isoindoline-1,3-dione (0.30 g, 1.09 mmol) and 2-[2-(2-aminoethoxy)ethoxy]ethanol (0.162 g, 1.09 mmol) in DMF (3 mL) was added DIPEA (0.31 mL, 2.17 mmol) and the resulting mixture was stirred at 80° C. for 16 h. The reaction was confirmed to completion by LCMS. The reaction mixture was cooled to room temperature, EtOAc (30 mL) and water (30 mL) were added and layers were separated. The organic phase was washed with water (10 mL×2) and brine (100 mL), dried over Na₂SO₄, filtered and the filtrate was concentrated under reduced pressure. The residue was purified by silica gel chromatography (Dichloromethane:Methanol=9:1) to obtain the title compound as a yellow oil (164 mg, 0.404 mmol, 37% yield).

MS(M+H)⁺=406.1

### Step 8. Synthesis of 2-(2,6-dioxopiperidin-3-yl)-4-((2-(2-(2-(2-hydroxyethoxy)ethoxy)ethoxy)ethyl)amino)isoindoline-1,3-dione (5c)

The title compound (860 mg, 52% yield) was obtained as a yellow oil in a similar manner to the synthesis scheme of Compound 5a.

MS(M+H)⁺=450.1

### Step 9. Synthesis of 2-(2,6-dioxopiperidin-3-yl)-4-((14-hydroxy-3,6,9,12-tetraoxatetradecyl)amino)isoindoline-1,3-dione (5d)

To a solution of 2-(2,6-dioxo-3-piperidyl)-4-fluoro-isoindoline-1,3-dione (1.00 g, 3.62 mmol) and 14-amino-3,6,9,12-tetraoxatetradecan-1-ol (0.902 g, 3.80 mmol) in DMF (10 mL) was added DIPEA (1.03 mL, 7.24 mmol) and the resulting mixture was stirred at 80° C. for 16 h. The reaction was confirmed to completion by LCMS. The reaction mixture was cooled to room temperature, EtOAc (100 mL) and water (100 mL) were added and layers were separated. The organic phase was washed with water (50 mL×2) and brine (100 mL), dried over Na₂SO₄, filtered and the filtrate was concentrated under reduced pressure. The residue was purified by silica gel chromatography (Dichloromethane:Methanol=9:1) to obtain the title compound as a yellow oil (388 mg, 0.787 mmol, 22% yield).

MS(M+H)⁺=494.2

### <Example 5> Synthesis of N-(2-(2-((2-(2,6-dioxopiperidin-3-yl)-1,3-dioxoisoindolin-4-yl)amino)ethoxy)ethyl)-3-(N-((1,2,3,5,6,7-hexahydro-s-indacen-4-yl)carbamoyl)sulfamoyl)-1-isopropyl-1H-pyrazol-5-carboxamide (Compound 5)

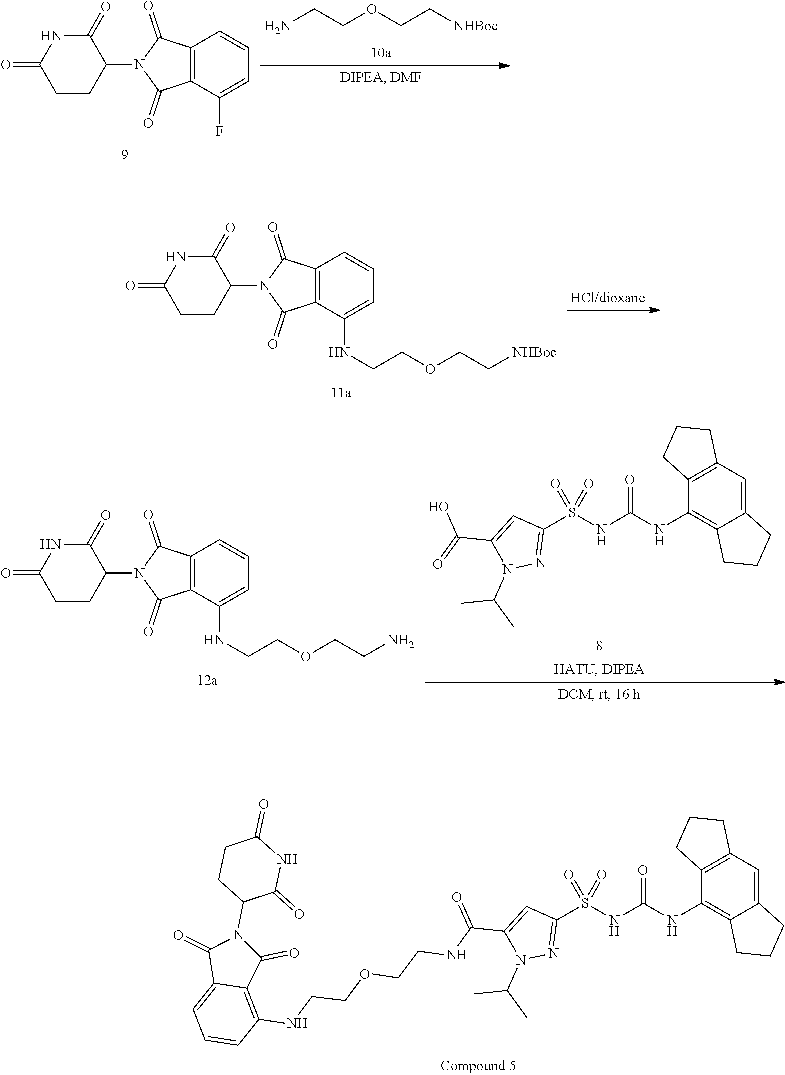

### Step 1. Synthesis of ethyl-3-nitro-1H-pyrazol-5-carboxylate (2)

To a solution of 3-nitro-1H-pyrazol-5-carboxylic acid (5.0 g, 31.83 mmol) in EtOH (41 mL), SOCl₂ (8.9 mL, 127.32 mmol) was added, and the mixture was stirred at 70° C. for 6 hours. The main peak of the desired mass was confirmed by LCMS. After the reaction mixture was concentrated under reduced pressure, water (100 mL) was poured into the residue, followed by extraction with EtOAc (200 mL×3). The organic phase was washed with sat. NaHCO₃ (100 mL×3), dried over Na₂SO₄, filtered and concentrated under reduced pressure. The residue was purified by silica gel column chromatography (Hex:EtOAc=100:0 to 95:5) to obtain the title compound as a brown solid (5 g, 84% yield).

MS[M+H]=186.1

### Step 2. Synthesis of ethyl-1-isopropyl-3-nitro-1H-pyrazol-5-carboxylate (3)

To a solution of ethyl-3-nitro-1H-pyrazol-5-carboxylate (2.0 g, 10.80 mmol) in DMF (50 mL), 2-iodopropane (1.30 mL, 12.96 mmol) and K₂CO₃ (2.90 g, 21.60 mmol) were added and stirred at 90° C. for 4 hours. The main peak of the desired mass was confirmed by LCMS. Water (100 mL) was poured into the mixture, extracted with EtOAc (20 mL×3), dried over Na₂SO₄, filtered, and concentrated under reduced pressure. The residue was purified by silica gel column chromatography (Hex:EtOAc=100:0 to 90:10) to obtain the title compound as a yellow solid (700 mg, 30% yield).

MS[M+H]=228.1

### Step 3. Synthesis of ethyl-3-amino-1-isopropyl-1H-pyrazol-5-carboxylate (4)

To a solution of ethyl-1-isopropyl-3-nitro-1H-pyrazol-5-carboxylate (700 mg, 3.08 mmol) in MeOH (15 mL), 5% Pd/C (3.0 g, 30.80 mmol) was added. The mixture was degassed and purged with N₂ three times. The resulting mixture was stirred at 25° C. under hydrogen gas for 6 hours. The main peak of the desired mass was confirmed by LCMS. The reaction mixture was filtered through celite and concentrated under reduced pressure. Water (50 mL) was poured into the residue, followed by extraction with EtOAc (50 mL×3), and then the organic phase was washed with sat. NaHCO₃ (50 mL), dried over Na₂SO₄, filtered, and concentrated under reduced pressure. The white title compound was obtained (510 mg, 84% yield).

MS[M+H]=198.1

### Step 4. Synthesis of ethyl-1-isopropyl-3-sulfamoyl-1H-pyrazol-5-carboxylate (5)

Ethyl-3-amino-1-isopropyl-1H-pyrazol-5-carboxylate (510 mg, 2.58 mmol) was placed in a two-necked flask and dissolved in ACN (32 mL). To the resulting mixture, conc HCl (5.8 mL) and NaNO₂ (214 mg, 3.10 mmol) were added and stirred at 0° C. for 1 hour. After adding AcOH (5.8 mL) and CuCl₂·2H₂O (441 mg, 2.58 mmol) to the reaction mixture, the mixture was stirred for 3 hours at 0° C. under SO₂ gas. The main peak of the desired intermediate mass was confirmed by LCMS. The reaction mixture was quenched by adding H₂O (500 mL) and extracted with DCM (200 mL×3). The extract was dried over Na₂SO₄, filtered, and concentrated under reduced pressure. The residue was dissolved in acetone, and NH₄OH was added, followed by stirring at 0° C. for 30 minutes. The main peak of the desired mass was confirmed by LCMS. After concentration of the reaction mixture in vacuo, water (50 mL) was poured into the residue, extracted with EtOAc (50 mL×3), dried over Na₂SO₄, filtered, and concentrated under reduced pressure. The residue was purified by silica gel column chromatography (Hex:EtOAc=100:0 to 70:30) to obtain the title compound as a white solid (110 mg, 16% yield).

MS[M+H]=262.0

### Step 5. Synthesis of ethyl-3-(N-((1,2,3,5,6,7-hexahydro-s-indacen-4-yl)carbamoyl)sulfamoyl)-1-isopropyl-1H-pyrazol-5-carboxylate (7)

To a solution of di-tert-butyl dicarbonate (135 μL, 0.59 mmol) in ACN (2 mL), DMAP (36 mg, 0.29 mmol) was added, and stirred at 25° C. for 30 minutes. To the resulting reaction mixture, 1,2,3,5,6,7-hexahydro-s-indacen-4-amine (73 mg, 0.42 mmol) was added. In another flask, ethyl-1-isopropyl-3-sulfamoyl-1H-pyrazol-5-carboxylate (110 mg, 0.42 mmol) was dissolved in ACN (2 mL), and then NaOMe (23 mg, 0.42 mmol) was added. The resulting reaction mixture was added to a mixture of di-tert-butyl dicarbonate and 1,2,3,5,6,7-hexahydro-s-indacen-4-amine, followed by stirring at 25° C. for 16 hours. The main peak of the desired mass was confirmed by LCMS. Water (10 mL) was poured into the mixture, extracted with EtOAc (10 mL×3), dried over Na₂SO₄, filtered, and concentrated under reduced pressure to obtain the title compound in the form of a brown foam (230 mg, crude).

MS[M+H]=461.1

### Step 6. Synthesis of 3-(N-((1,2,3,5,6,7-hexahydro-s-indacen-4yl)carbamoyl)sulfamoyl)-1-isopropyl-1H-pyrazol-5-carboxylic acid (8)

To a solution of ethyl-3-(N-((1,2,3,5,6,7-hexahydro-s-indacen-4-yl)carbamoyl)sulfamoyl)-1-isopropyl-1H-pyrazol-5-carboxylate (230 mg, 0.49 mmol) in THF/MeOH (0.4 mL:0.1 mL), LiOH·H₂O (25 mg, 0.60 mmol) in water (0.2 mL) was added and the resulting mixture was stirred at 25° C. for 3 hours. The main peak of the desired mass was confirmed by LCMS. The reaction mixture was concentrated in vacuo. The residue was acidified to pH 3 with 1M HCl. The title compound was obtained as a white solid (122 mg, 67% in 2 step yield).

MS[M+H]=433.1

### Step 7. Synthesis of tert-butyl(2-(2-((2-(2,6-dioxopiperidin-3-yl)-1,3-dioxoisoindolin-4-yl)amino)ethoxy)ethyl)carbamate (11a)

To a solution of 2-(2,6-dioxo-3-piperidyl)-4-fluoro-isoindoline-1,3-dione (1.01 g, 3.65 mmol, 1 eq) and tert-butyl (2-(2-aminoethoxy)ethyl) carbamate (0.75 g, 3.65 mmol) in DMF (8 mL), DIPEA (7.30 mmol, 1.04 mL) was added, and the mixture was stirred at 90° C. for 12 hours. The main peak of the desired mass was confirmed by LCMS. Water (20 mL) was poured into the mixture, followed by extraction with EtOAc (20 mL×3), and the organic phase was washed with 1N HCl (10 mL×3), and brine (10 mL×2), and concentrated under reduced pressure. The residue was purified by silica gel column chromatography (Hex:EtOAc=1:1 to 1:3) to obtain the title compound as a black oil (0.472 g, 1.02 mmol, 28% yield).

MS[M+Na]=483.1

### Step 8. Synthesis of 4-((2-(2-aminoethoxy)ethyl)amino)-2-(2,6-dioxopiperidin-3-yl)isoindoline-1,3-dione (12a)

To a solution of tert-butyl(2-(2-((2-(2,6-dioxopiperidin-3-yl)-1,3-dioxoisoindolin-4-yl)amino)ethoxy)ethyl)carbamate (0.472 g, 1.02 mmol) in dioxane (5 mL), HCl/dioxane (4 M, 0.513 mL) was added, and the resulting mixture was stirred at 25° C. for 4 hours. The main peak of the desired mass was confirmed by LCMS. The reaction mixture was concentrated in vacuo to obtain the title compound as a brown solid (0.410 g, crude, HCl salt).

MS[M+H]=361.1

### Step 9. Synthesis of N-(2-(2-((2-(2,6-dioxopiperidin-3-yl)-1,3-dioxoisoindolin-4-yl)amino)ethoxy)ethyl)-3-(N-((1,2,3,5,6,7-hexahydro-s-indacen-4-yl)carbamoyl)sulfamoyl)-1-isopropyl-1H-pyrazol-5-carboxamide (Compound 5)

To a solution of 3-(N-((1,2,3,5,6,7-hexahydro-s-indacen-4yl)carbamoyl)sulfamoyl)-1-isopropyl-1H-pyrazol-5-carboxylic acid (100 mg, 0.23 mmol) in DCM (5 mL), HATU (96 mg, 0.25 mmol), DIPEA (201 μL, 0.58 mmol) and 4-((2-(2-aminoethoxy)ethyl)amino)-2-(2,6-dioxopiperidin-3-yl)isoindoline-1,3-dione (110 mg, 0.28 mmol) were added. The resulting mixture was stirred at room temperature for 3 hours. The main peak of the desired mass was confirmed by LCMS. Water (10 mL) was poured into the mixture, extracted with DCM (10 mL×3), dried over Na₂SO₄, and filtered. The residue was purified by silica gel column chromatography (DCM:MeOH=100:0 to 90:10) to obtain the yellow title compound (36 mg, 46.4 μmol, 96% purity).

¹H NMR (400 MHz, DMSO-d₆) δ=11.10 (s, 1H), 8.78 (brs, 1H) 8.19 (brs, 1H), 7.58-7.54 (m, 1H), 7.14 (d, J=8.4 Hz, 1H), 7.02 (d, J=7.0 Hz, 1H), 6.90 (brs, 1H), 6.62 (t, J=6.0 Hz, 1H), 5.54-5.50 (m, 1H), 5.05 (dd, J=12.5, 5.4 Hz, 1H), 3.65-3.56 (m, 8H), 3.48-3.46 (m, 2H), 3.17-3.13 (m, 2H), 2.78-2.75 (m, 4H), 2.60-2.56 (m, 4H), 1.93-1.89 (m, 4H), 1.39 (s, 3H), 1.38 (s, 3H).

MS[M+H]=775.2

### <Example 6> Synthesis of N-(2-(2-(2-((2-(2,6-dioxopiperidin-3-yl)-1,3-dioxoisoindolin-4-yl)amino)ethoxy)ethoxy)ethyl)-3-(N-((1,2,3,5,6,7-hexahydro-s-indacen-4-yl)carbamoyl)sulfamoyl)-1-isopropyl-1H-pyrazol-5-carboxamide (Compound 6)

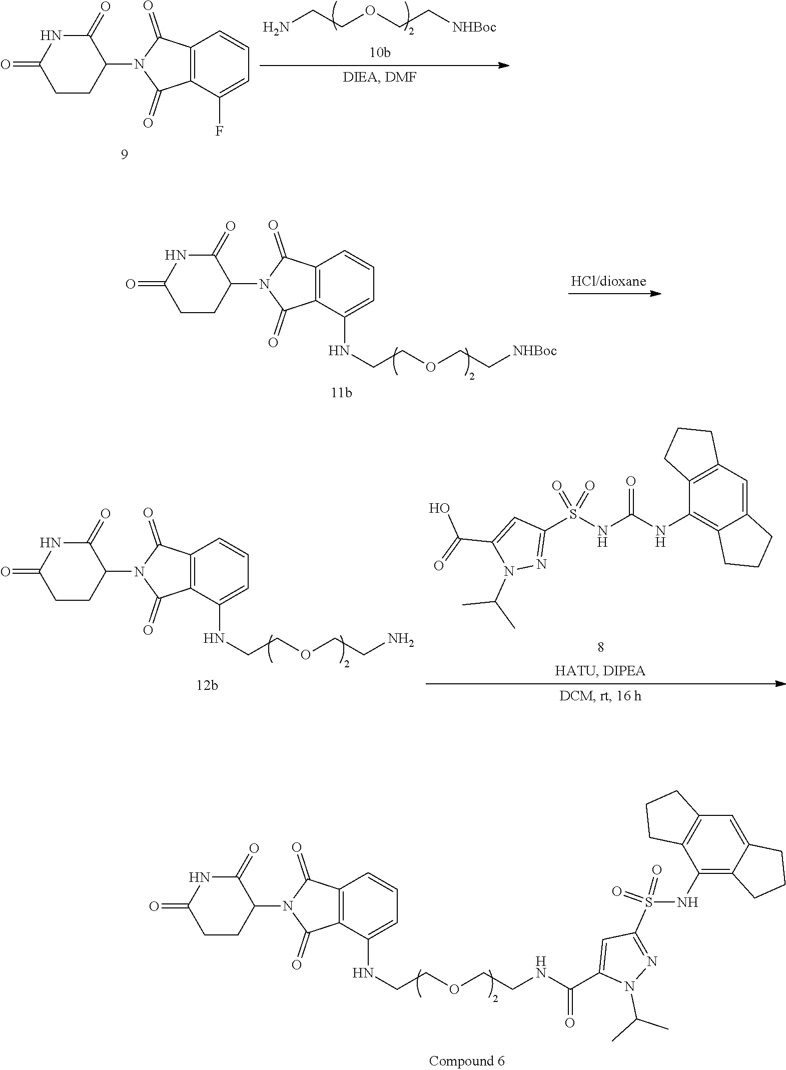

### Step 1. Synthesis of tert-butyl(2-(2-(2-((2-(2,6-dioxopiperidin-3-yl)-1,3-dioxoisoindolin-4-yl)amino)ethoxy)ethoxy)ethyl)carbamate (11b)

The title compound (1.05 g, 2.07 mmol, 40% yield) was obtained as a black oil in a similar manner to Step 7 of the synthesis scheme of Compound 1.

MS[M+Na]=527.2

### Step 2. Synthesis of 4-((2-(2-aminoethoxy)ethyl)amino)-2-(2,6-dioxopiperidin-3-yl)isoindoline-1,3-dione (12b)

The title compound (880 mg, crude, HCl salt) was obtained as a brown solid in a similar manner to Step 8 of the synthesis scheme of Compound 1.

MS[M+H]=405.2

### Step 3. Synthesis of N-(2-(2-(2-((2-(2,6-dioxopiperidin-3-yl)-1,3-dioxoisoindolin-4-yl)amino)ethoxy)ethoxy)ethyl)-3-(N-((1,2,3,5,6,7-hexahydro-s-indacen-4-yl)carbamoyl)sulfamoyl)-1-isopropyl-1H-pyrazol-5-carboxamide (Compound 6)

To a solution of 3-(N-((1,2,3,5,6,7-hexahydro-s-indacen-4yl)carbamoyl)sulfamoyl)-1-isopropyl-1H-pyrazol-5-carboxylic acid (50 mg, 0.11 mmol) in DCM (1 mL), HATU (48 mg, 0.13 mmol), DIPEA (100 μL, 0.58 mmol) and 4-((2-(2-(2-aminoethoxy)ethoxy)ethyl)amino)-2-(2,6-dioxopiperidin-3-yl)isoindoline-1,3-dione (76 mg, 0.17 mmol) were added. The resulting mixture was stirred at room temperature for 1 hour. The main peak of the desired mass was confirmed by LCMS. Water (10 mL) was poured into the mixture, extracted with DCM (10 mL×3), dried over Na₂SO₄, and filtered. The residue was purified by silica gel column chromatography (Hex:EtOAc=100:0 to 0:100, DCM:MeOH=100:0 to 90:10) to obtain the yellow title compound (40.0 mg, 48.8 μmol, 42% yield, 93% purity).

¹H NMR (600 MHz, DMSO-d₆) δ=11.08 (s, 1H), 10.2 (s, 1H), 7.95 (s, 1H), 7.59-7.53 (m, 1H), 7.14-7.11 (m, 1H), 7.04-7.02 (m, 1H), 6.80 (brs, 1H), 6.60-6.58 (m, 1H), 6.33 (s, 1H), 5.54-5.50 (m, 1H), 5.04 (dd, J=12.8, 5.5 Hz, 1H), 3.62-3.55 (m, 4H), 3.53-3.50 (m, 6H), 3.32-3.30 (m, 6H), 2.90-2.86 (m, 2H), 2.75-2.71 (m, 2H), 2.64-2.57 (m, 4H), 2.03-1.86 (m, 4H), 1.39 (s, 3H), 1.36 (s, 3H).

MS[M+H]=819.2

### <Example 7> Synthesis of N-(2-(2-(2-(2-((2-(2,6-dioxopiperidin-3-yl)-1,3-dioxoisoindolin-4-yl)amino)ethoxy)ethoxy)ethoxy)ethyl)-3-(N-((1,2,3,5,6,7-hexahydro-s-indacen-4-yl)carbamoyl)sulfamoyl)-1-isopropyl-1H-pyrazol-5-carboxamide (Compound 7)

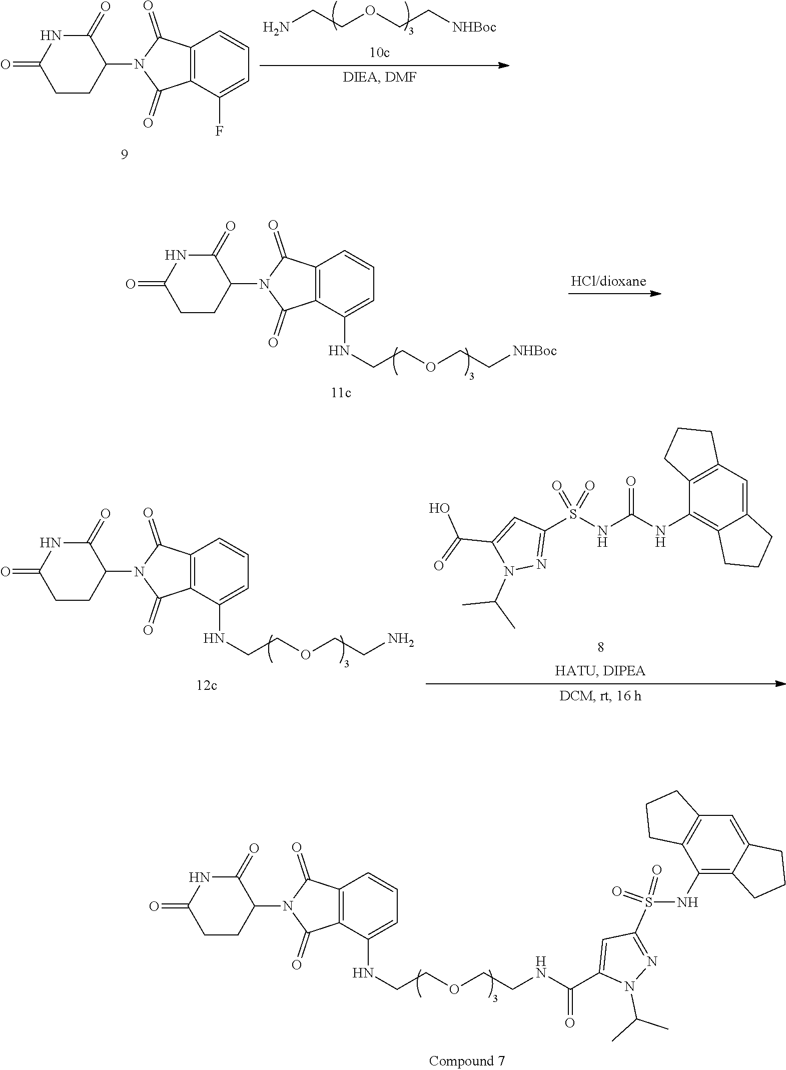

### Step 1. Synthesis of tert-butyl(2-(2-(2-(2-(2-(2-(2,6-dioxopiperidin-3-yl)-1,3-dioxoisoindolin-4-yl)amino)ethoxy)ethoxy)ethoxy)ethyl)carbamate (11c)

The title compound (916 mg, 1.67 mmol, 41% yield) was obtained as a black oil in a similar manner to Step 7 of the synthesis scheme of Compound 1.

MS[M+Na]=571.2

### Step 2. Synthesis of 4-((2-(2-(2-(2-(2-(2-aminoethoxy)ethoxy)ethyl)amino)-2-(2,6-dioxopiperidin-3-yl)isoindoline-1,3-dione (12c)

The title compound (773 mg, crude, HCl salt) was obtained as a brown oil in a similar manner to Step 8 of the synthesis scheme of Compound 1.

MS[M+H]=449.1

### Step 3. Synthesis of N-(2-(2-(2-(2-((2-(2,6-dioxopiperidin-3-yl)-1,3-dioxoisoindolin-4-yl)amino)ethoxy)ethoxy)ethoxy)ethyl)-3-(N-((1,2,3,5,6,7-hexahydro-s-indacen-4-yl)carbamoyl)sulfamoyl)-1-isopropyl-1H-pyrazol-5-carboxamide (Compound 7)

To a solution of 3-(N-((1,2,3,5,6,7-hexahydro-s-indacen-4yl)carbamoyl)sulfamoyl)-1-isopropyl-1H-pyrazol-5-carboxylic acid (30 mg, 0.069 mmol) in DCM (1 mL), HATU (29 mg, 0.0763 mmol), DIPEA (18 μL, 0.10 mmol) and 4-((2-(2-(2-aminoethoxy)ethoxy)ethoxy)ethyl)amino)-2-(2,6-dioxopiperidin-3-yl)isoindoline-1,3-dione (40 mg, 0.083 mmol) were added. The resulting mixture was stirred at room temperature for 1 hour. The main peak of the desired mass was confirmed by LCMS. Water (10 mL) was poured into the mixture, extracted with DCM (10 mL×3), dried over Na₂SO₄, and filtered. The residue was purified by silica gel column chromatography (DCM:MeOH=100:0 to 80:20) to obtain the yellow title compound (2.0 mg, 2.3 μmol, 3.4% yield, 90% purity).

MS[M+H]=863.2

### <Example 8> Synthesis of N-(14((2-(2,6-dioxopiperidin-3-yl)-1,3-dioxoisoindolin-4-yl)amino)-3,6,9,23-tetraoxatetradecyl)-3-(N-((1,2,3,5,6,7-hexahydro-s-indacen-4-yl)carbamoyl)sulfamoyl)-1-isopropyl-1H-pyrazol-5-carboxamide (Compound 8)

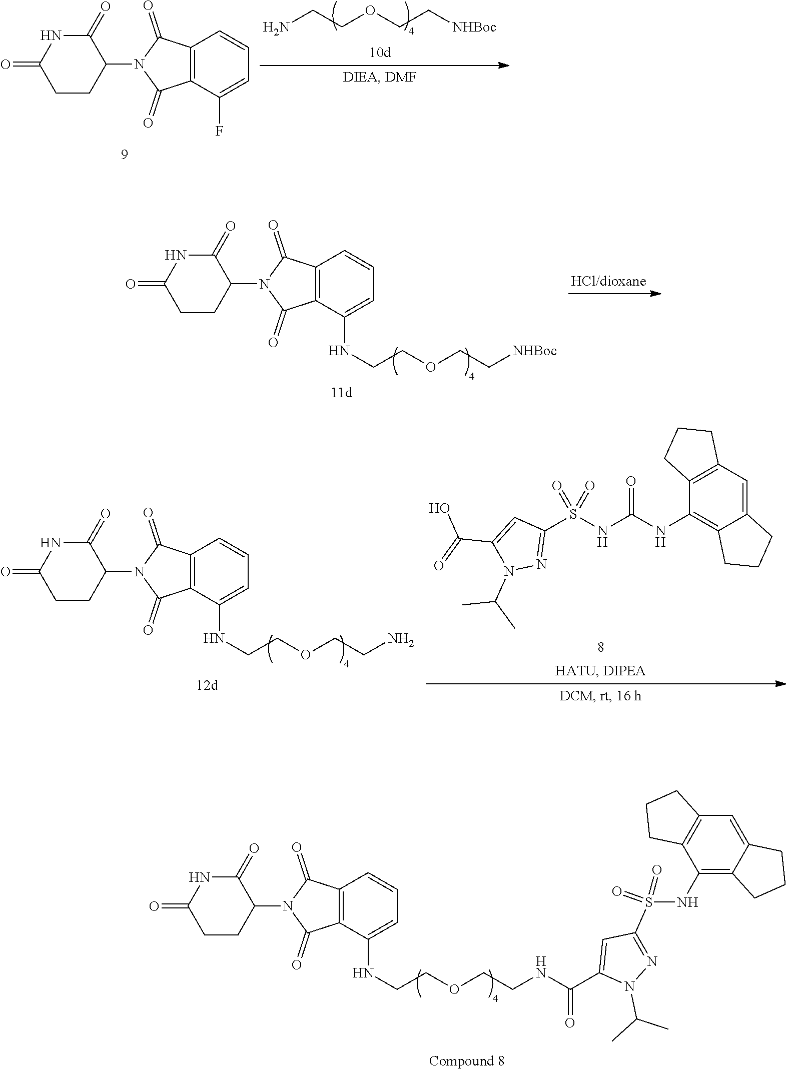

### Step 1. Synthesis of tert-butyl (14-(2-(2,6-dioxopiperidin-3-yl)-1,3-dioxoisoindolin-4-yl)amino)-3,6,9,12-tetraoxatetradecyl)carbamate (11d)

The title compound (0.8 g, 1.35 mmol, 22% yield) was obtained as a black oil in a similar manner to Step 7 of the synthesis scheme of Compound 1.

MS[M+H]=593.2

### Step 2. Synthesis of 4-(14-amino-3,6,9,12-tetraoxatetradecyl)amino)-2-(2,6-dioxopiperidin-3-yl) isoindoline-1,3-dione (12d)

The title compound (0.33 g, 0.37 mmol, 46% yield, HCl salt) was obtained as a brown oil in a similar manner to Step 8 of the synthesis scheme of Compound 1.

MS[M+H]=493.2

### Step 3. Synthesis of N-(14((2-(2,6-dioxopiperidin-3-yl)-1,3-dioxoisoindolin-4-yl)amino)-3,6,9,23-tetraoxatetradecyl)-3-(N-((1,2,3,5,6,7-hexahydro-s-indacen-4-yl)carbamoyl)sulfamoyl)-1-isopropyl-1H-pyrazol-5-carboxamide (Compound 8)

To a solution of 3-(N-((1,2,3,5,6,7-hexahydro-s-indacen-4yl)carbamoyl)sulfamoyl)-1-isopropyl-1H-pyrazol-5-carboxylic acid (40 mg, 0.092 mmol) in DCM (1 mL), HATU (38 mg, 0.10 mmol), DIPEA (24 μL, 0.14 mmol) and 4-((14-amino-3,6,9,12-tetraoxatetradecyl)amino-2-(2,6-dioxopiperidin-3-yl)isoindoline-1,3-dione (54 mg, 0.11 mmol) were added. The resulting mixture was stirred at room temperature for 4 hours. The main peak of the desired mass was confirmed by LCMS. Water (10 mL) was poured into the mixture, extracted with DCM (10 mL×3), dried over Na₂SO₄, and filtered. The residue was purified by silica gel column chromatography (DCM:MeOH=100:0 to 90:10) to obtain the yellow title compound (4.0 mg, 4.4 μmol, 5% yield, 93% purity).

¹H NMR (400 MHz, DMSO-d₆) δ=11.10 (s, 1H), 8.78 (brs, 1H), 7.60-7.56 (m, 1H), 7.16 (d, J=8.5 Hz, 1H), 7.05 (d, J=7.1 Hz, 1H), 6.90 (brs, 1H), 6.61 (t, J=6.4 Hz, 1H), 5.54-5.50 (m, 1H), 5.05 (dd, J=12.5, 5.4 Hz, 1H), 3.65-3.46 (m, 22H), 3.17-3.13 (m, 2H), 2.76-2.74 (m, 4H), 2.60-2.51 (m, 4H), 1.92-1.89 (m, 4H), 1.39 (s, 3H), 1.38 (s, 3H).

MS[M+H]=907.2

### <Example 9> Synthesis of 3-(N-((1,2,3,5,6,7-hexahydro-s-indacen-4-yl)carbamoyl)sulfamoyl)-N—((S)-13-((2S,4R)-4-hydroxy-2-((4-(4-methylthiazol-5-yl)benzyl)carbamoyl)pyrrolidine-1-carbonyl)-14,14-dimethyl-11-oxo-3,6,9-trioxa-12-azapentadecyl-1-isopropyl-1H-pyrazol-5-carboxamide (Compound 9)

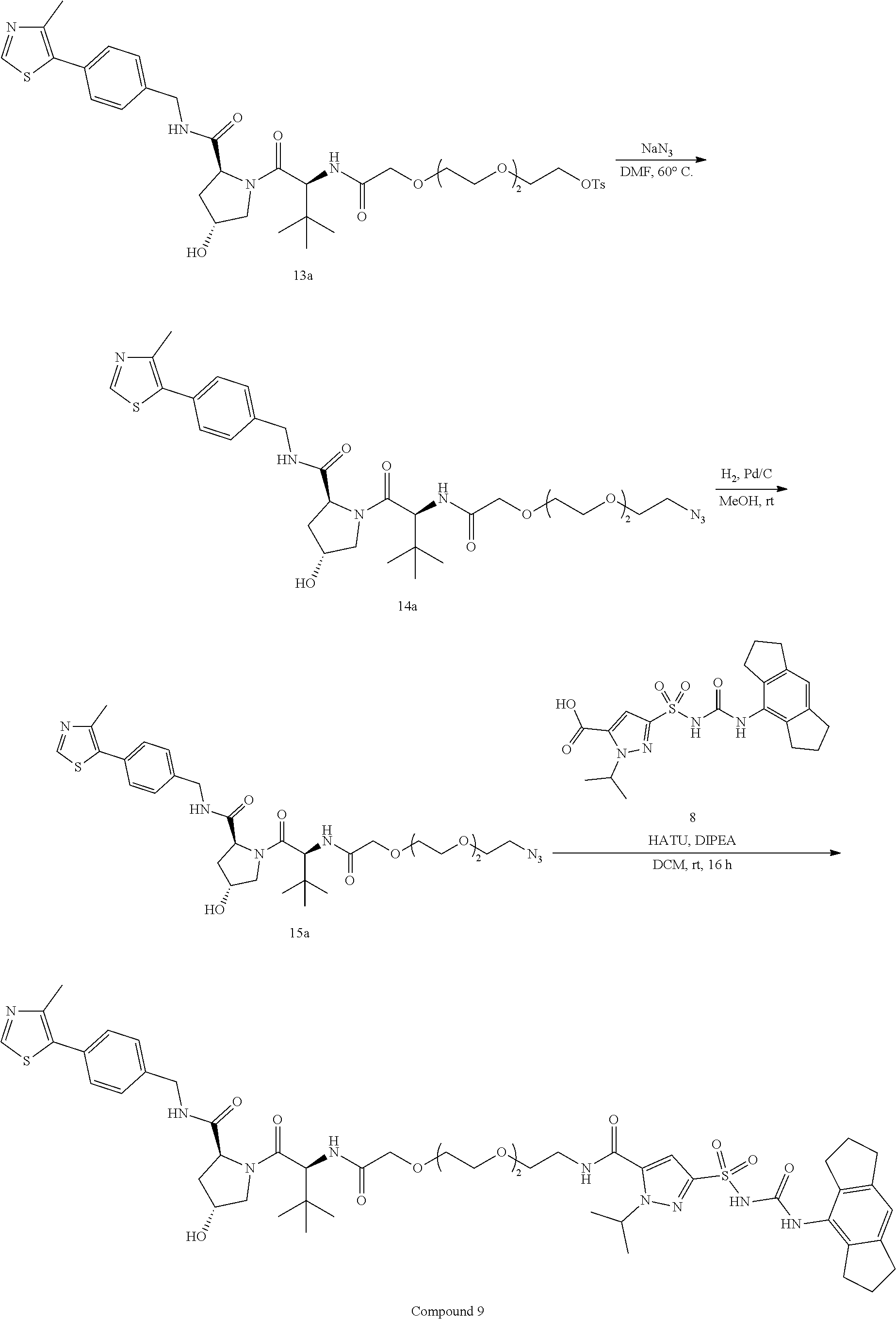

### Step 1. Synthesis of (2S,4R)-1-((S)-14-azido-2-(tert-butyl)4-oxo-6,9,12-trioxa-3-azatetradecanoyl)-4-hydroxy-N-(4-(4-methylthiazol-5-yl)benzyl)pyrrolidine-2-carboxamide (14a)

To a solution of (S)-13-((2S,4R)-4-hydroxy-2-((4-(4-methylthiazol-5-yl)benzyl)carbamoyl)pyrrolidine-1-carbonyl)-14,14-dimethyl-11-oxo-3,6,9-trioxa-12-azapentadecyl-4-methylbenzenesulfonate (1.00 g, 1.37 mmol) in DMF (10 mL), NaN₃ (196 mg, 3.01 mmol) was added in one portion at 25° C. and the reaction mixture was stirred at 60° C. for 8 hours. Complete consumption of the starting material was confirmed by LCMS and the desired mass was detected. The mixture was diluted with H₂O (10 mL) and extracted with EtOAc (10 mL×3). The organic layer was washed with brine (10 mL), dried over Na₂SO₄, filtered and concentrated under reduced pressure to obtain the title compound as a yellow oil (850 mg, crude).

### Step 2. Synthesis of (2S,4R)-1-((S)-14-amino-2-(tert-butyl)-4-oxo-6,9,12-trioxa-3-azatetradecanoyl)-4-hydroxy-N-(4-(4-methylthiazol-5-yl)benzyl)pyrrolidine-2-carboxamide (15a)

To a solution of (2S,4R)-1-((S)-14-azido-2-(tert-butyl)4-oxo-6,9,12-trioxa-3-azatetradecanoyl)-4-hydroxy-N-(4-(4-methylthiazol-5-yl)benzyl)pyrrolidine-2-carboxamide (850 mg, crude) in MeOH (10 mL), Pd/C (200 mg, 20 wt %, 5% purity) was added. The mixture was then degassed and purged with N₂ three times. The reaction mixture was stirred at room temperature for 16 hours under H₂ atmosphere. The reaction mixture was filtered and concentrated under reduced pressure to obtain the title compound as a colorless oil (780 mg, crude).

MS[M+H]=620.2

### Step 3. Synthesis of 3-(N-((1,2,3,5,6,7-hexahydro-s-indacen-4-yl)carbamoyl)sulfamoyl)-N—((S)-13-((2S,4R)-4-hydroxy-2-((4-(4-methylthiazol-5-yl)benzyl)carbamoyl)pyrrolidine-1-carbonyl)-14,14-dimethyl-11-oxo-3,6,9-trioxa-12-azapentadecyl-1-isopropyl-1H-pyrazol-5-carboxamide (Compound 9)

To a solution of 3-(N-((1,2,3,5,6,7-hexahydro-s-indacen-4yl)carbamoyl)sulfamoyl)-1-isopropyl-1H-pyrazol-5-carboxylic acid (60 mg, 0.14 mmol) in DCM (1 mL), HATU (58 mg, 0.15 mmol), DIPEA (36 μL, 0.21 mmol) and (2S,4R)-1-((S)-14-amino-2-(tert-butyl)-4-oxo-5,9,12-trioxa-3-azatetradecanoyl)-4-hydroxy-N-(4-(4-methylthiazol-5-yl)benzyl)pyrrolidine-2-carboxamide (54 mg, 0.11 mmol) were added. The resulting mixture was stirred at room temperature for 3 hours. The main peak of the desired mass was confirmed by LCMS. Water (10 mL) was poured into the mixture, extracted with DCM (10 mL×3), dried over Na₂SO₄, and filtered. The residue was purified by silica gel column chromatography (EtOAc:MeOH=100:0 to 90:10) to obtain the white title compound (56 mg, 54.1 μmol, 37% yield, 92% purity).

¹H NMR (400 MHz, DMSO-d₆) δ=8.98 (s, 1H), 8.62 (t, J=5.6 Hz, 2H), 7.45-7.40 (m, 6H), 6.81 (brs, 1H), 5.53-5.48 (m, 1H), 5.17 (d, J=3.6 Hz, 1H), 4.58-4.56 (d, J=9.6 Hz, 1H), 4.47-4.40 (m, 2H), 4.38-4.36 (m, 2H), 4.28-4.27 (m, 1H), 3.96 (s, 2H), 3.66-3.47 (m, 14H), 2.77-2.71 (m, 4H), 2.64-2.58 (m, 4H), 2.45 (s, 3H), 1.94-1.88 (m, 5H), 1.38 (s, 3H), 1.36 (s, 3H), 0.95 (s, 9H).

MS[M+H]=1034.3

### <Example 10> Synthesis of 3-(N-((1,2,3,5,6,7-hexahydro-s-indacen-4-yl)carbamoyl)sulfamoyl)-N—((S)-16-((2S,4R)-4-hydroxy-2-((4-(4-methylthiazol-5-yl)benzyl)carbamoyl)pyrrolidine-1-carbonyl)17,17-dimethyl-14-oxo-3,6,9,12-tetraoxa-15-azaoctadecyl)-1-isopropyl-1H-pyrazol-5-carboxamide (Compound 10)

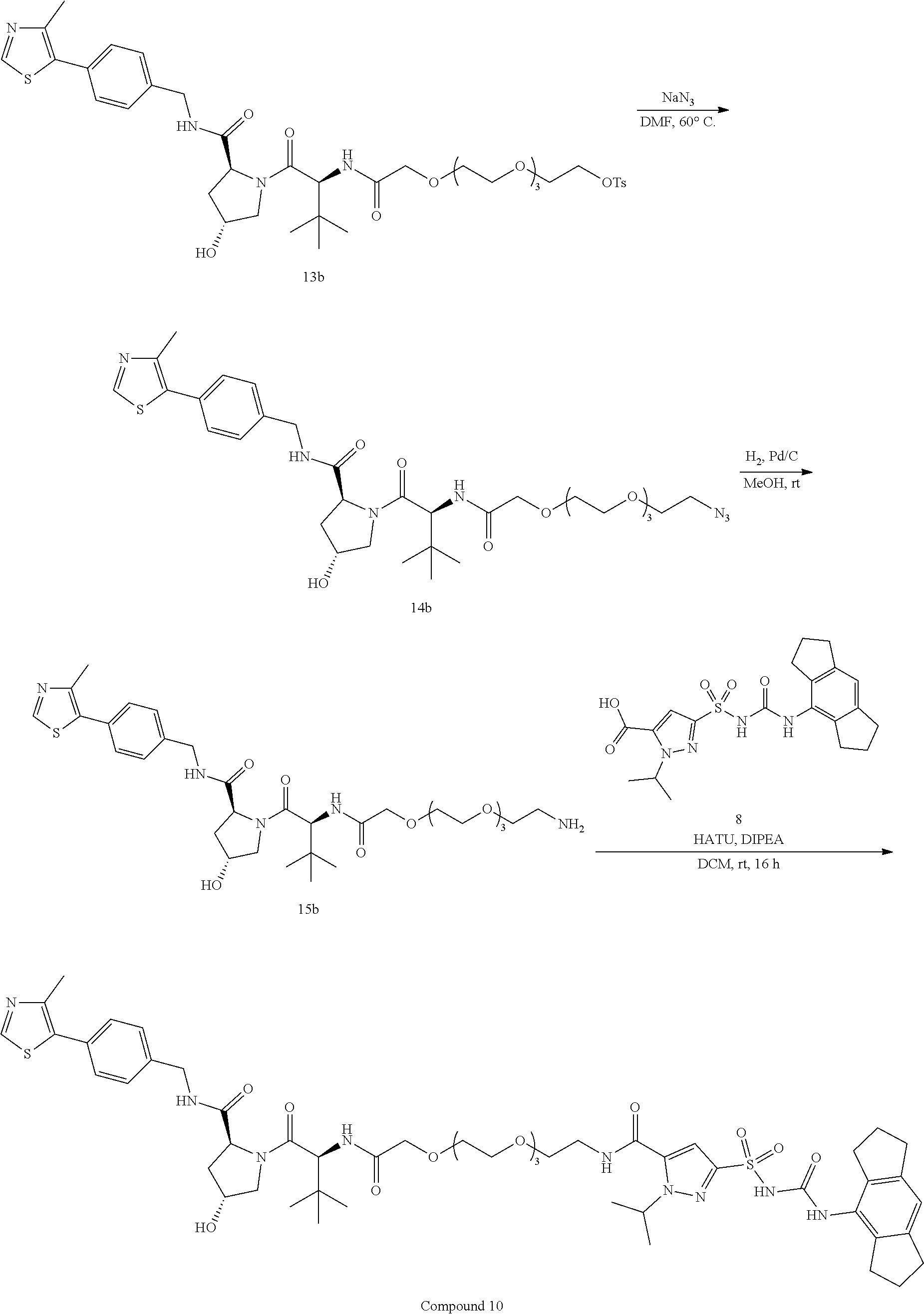

### Step 1. Synthesis of (2S,4R)-1-((S)-17-azido-2-(tert-butyl)-4-oxo-6,9,12,15-tetraoxa-3-azaheptadecanoyl)-4-hydroxy-N-(4-(4-methylthiazol-5-yl)benzyl)pyrrolidine-2-carboxamide (14b)

The title compound (800 mg, crude) was obtained as a yellow oil in a similar manner to Step 1 of the synthesis scheme of Compound 5.

### Step 2. Synthesis of (2S,4R)-1-((S)-17-amino-2-(tert-butyl)-4-oxo-6,9,12,15-tetraoxa-3-azaheptadecanoyl)-4-hydroxy-N-(4-(4-methylthiazol-5-yl)benzyl)pyrrolidine-2-carboxamide (15b)

The title compound (725 mg, crude) was obtained as a colorless oil in a similar manner to Step 2 of the synthesis scheme of Compound 5.

MS[M+H]=664.3

### Step 3. Synthesis of 3-(N-((1,2,3,5,6,7-hexahydro-s-indacen-4-yl)carbamoyl)sulfamoyl)-N—((S)-16-((2S,4R)-4-hydroxy-2-((4-(4-methylthiazol-5-yl)benzyl)carbamoyl)pyrrolidine-1-carbonyl)17,17-dimethyl-14-oxo-3,6,9,12-tetraoxa-15-azaoctadecyl)-1-isopropyl-1H-pyrazol-5-carboxamide (Compound 10)

To a solution of 3-(N-((1,2,3,5,6,7-hexahydro-s-indacen-4yl)carbamoyl)sulfamoyl)-1-isopropyl-1H-pyrazol-5-carboxylic acid (60 mg, 0.14 mmol) in DCM (1 mL), HATU (58 mg, 0.15 mmol), DIPEA (36 μL, 0.21 mmol), and (2S,4R)-1-((S)-17-amino-2-(tert-butyl)-4-oxo-6,9,12,15-tetraoxa-3-azaheptadecanoyl)-4-hydroxy-N-(4-(4-methylthiazol-5-yl)benzyl)pyrrolidine-2-carboxamide (73 ma, 0.11 mmol) were added. The resulting mixture was stirred at room temperature for 3 hours. The main peak of the desired mass was confirmed by LCMS. Water (10 mL) was poured into the mixture, extracted with DCM (10 mL×3), dried over Na₂SO₄, and filtered. The residue was purified by silica gel column chromatography (EtOAc:MeOH=100:0 to 90:10) to obtain the white title compound (56 mg, 54.1 μmol, 37% yield, 92% purity).

MS[M+H]=1078.3

### Total Synthesis of Compound 11˜Compound 12

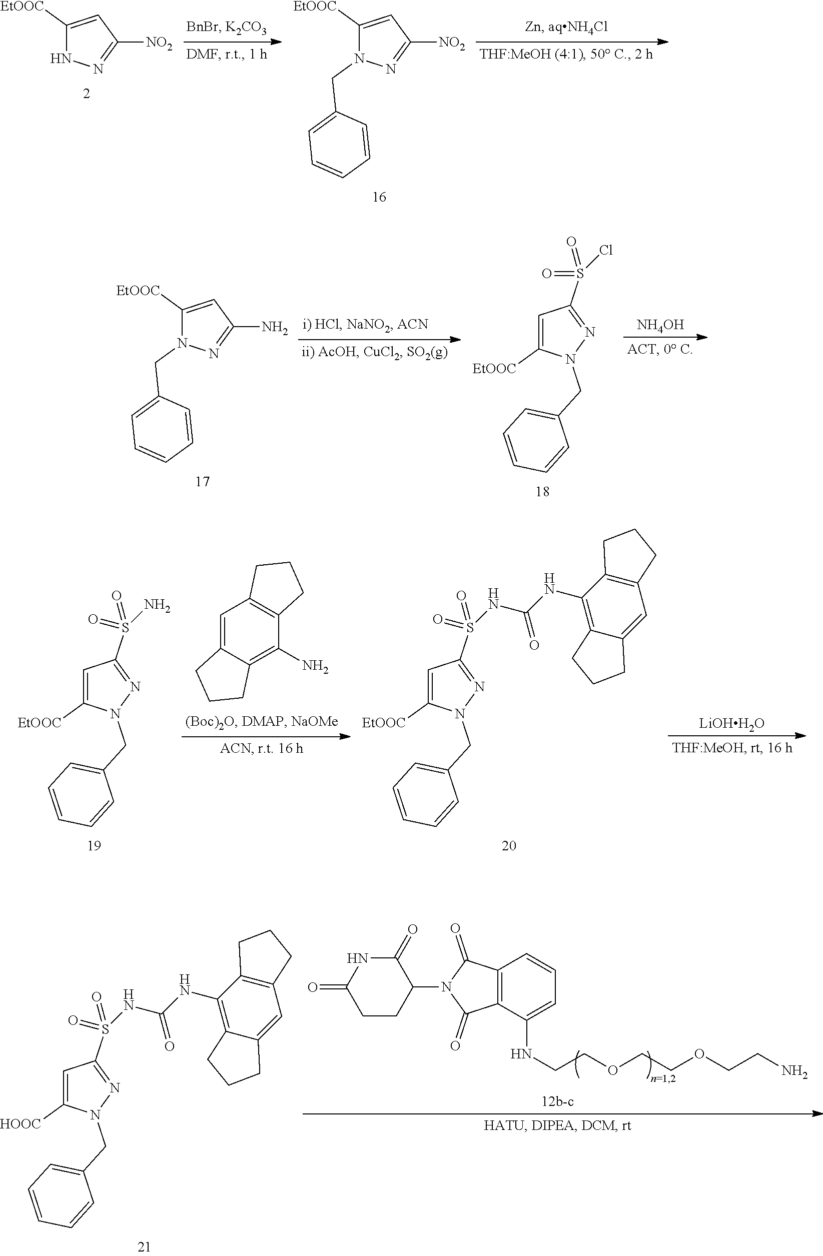

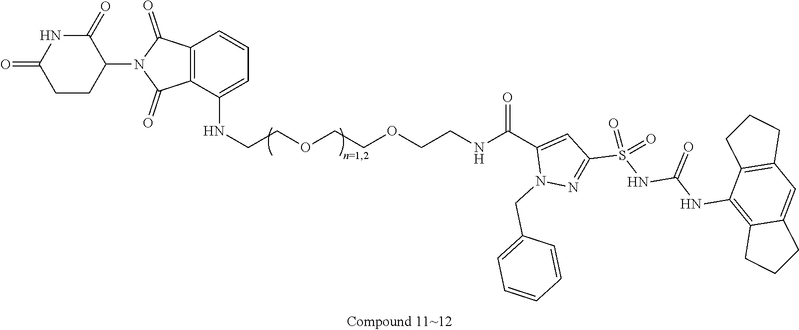

### <Example 11> Synthesis of 1-benzyl-N-(2-(2-(2-((2-(2,6-dioxopiperidin-3-yl)-1,3-dioxoisoindolin-4-yl)amino)ethoxy)ethoxy)ethyl)-3-(N-((1,2,3,5,6,7-hexahydro-s-indacen-4-yl)carbamoyl)sulfamoyl)-1H-pyrazol-5-carboxamide (Compound 11)

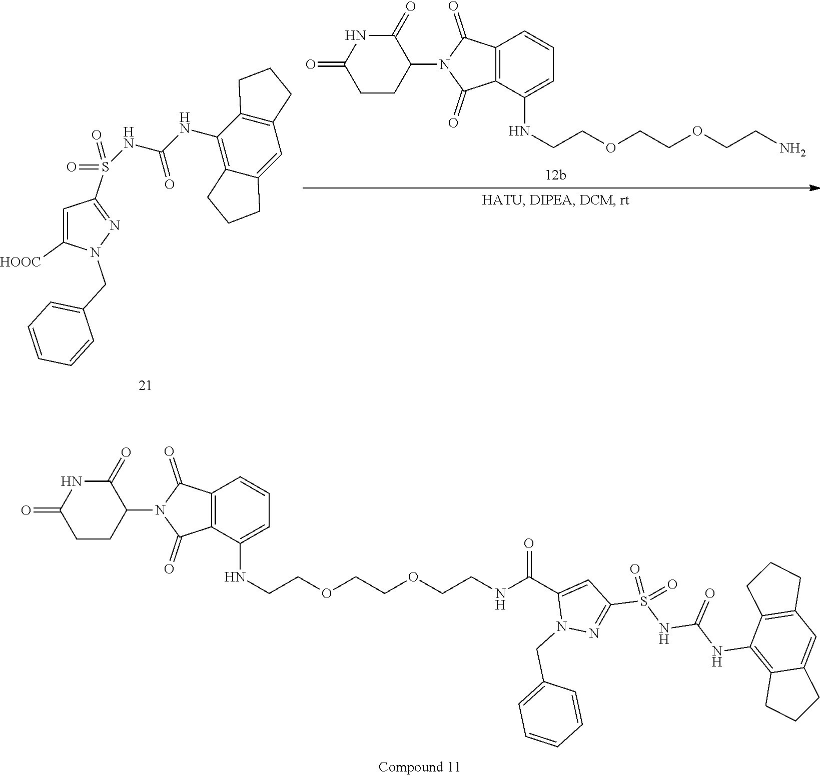

### Step 1. Synthesis of ethyl-1-benzyl-3-nitro-1H-pyrazol-5-carboxylate (16)

To a solution of ethyl-3-nitro-1H-pyrazol-5-carboxylate (1.63 g, 8.80 mmol) in DMF (29 mL), BnBr (1.25 mL, 10.56 mmol) and K₂CO₃ (1.82 g, 13.21 mmol) were added and stirred at 25° C. for 1 hour. The main peak of the desired mass was confirmed by LCMS. Water (100 mL) was poured into the mixture, extracted with EtOAc (20 mL×3), dried over Na₂SO₄, filtered, and concentrated under reduced pressure. The residue was purified by silica gel column chromatography (Hex:EtOAc=100:0 to 80:20) to obtain the title compound as a white solid (1.85 g, 76% yield).

MS[M+H]=276.6

### Step 2. Synthesis of ethyl-3-amino-1-benzyl-1H-pyrazol-5-carboxylate (17)

To a solution of ethyl-1-benzyl-3-nitro-1H-pyrazol-5-carboxylate (1.85 g, 6.73 mmol) in THF/MeOH (18 mL:4.5 mL), Zinc (2.20 g, 33.66 mmol) was added. NH₄Cl (1.80 g, 33.66 mmol) in H₂O (4.5 mL) was added to the resulting mixture and stirred at 50° C. for 2 hours. The main peak of the desired mass was confirmed by LCMS. The reaction mixture was filtered with celite using EtOAc and concentrated under reduced pressure. The residue was dissolved in EtOAc (1 mL), and Hex (10 mL) was added dropwise to precipitate a solid to obtain a pale yellow title compound (1.18 g, 71%).

MS[M+H]=246.5

### Step 3. Synthesis of ethyl-1-benzyl-3-sulfamoyl-1H-pyrazole-5-carboxylate (19)

Ethyl-3-amino-1-benzyl-1H-pyrazol-5-carboxylate (1.18 g, 4.81 mmol) was placed in a two-necked flask and dissolved in ACN (60 mL). To the resulting mixture, conc HCl (10.6 mL) and NaNO₂ (0.40 g, 5.83 mmol) were added and stirred at 0° C. for 1 hour. After adding AcOH (10.6 mL) and CuCl₂·2H₂O (0.82 g, 4.8 mmol) to the reaction mixture, the mixture was stirred for 3 hours at 0° C. under SO₂ gas. The main peak of the desired intermediate mass was confirmed by LCMS. The reaction mixture was quenched by adding H₂O (500 mL) and extracted with DCM (200 mL×3). The extract was dried over Na₂SO₄, filtered, and concentrated under reduced pressure. The residue was dissolved in acetone (34 mL), then NH₄OH (17.48 mL) was added and stirred at 0° C. for 30 minutes. The main peak of the desired mass was confirmed by LCMS. After concentration of the reaction mixture in vacuo, water (50 mL) was poured into the residue, extracted with EtOAc (50 mL×3), dried over Na₂SO₄, filtered, and concentrated under reduced pressure. The residue was purified by silica gel column chromatography (Hex:EtOAc=100:0 to 50:50) to obtain the title compound as a white solid (846 mg, 56% yield).

MS[M+H]=310.1

### Step 4. Synthesis of ethyl-1-benzyl-3-(N-((1,2,3,5,6,7-hexahydro-s-indacen-4-yl)carbamoyl)sulfamoyl)-1H-pyrazol-5-carboxylate (20)

To a solution of di-tert-butyl dicarbonate (0.88 mL, 3.8 mmol) in ACN (7 mL), DMAP (0.23 g, 1.91 mmol) was added, and stirred at 25° C. for 30 minutes. To the resulting reaction mixture, 1,2,3,5,6,7-hexahydro-s-indacen-4-amine (0.47 g, 2.74 mmol) was added. In another flask, ethyl-1-isopropyl-3-sulfamoyl-1H-pyrazol-5-carboxylate (0.85 g, 2.74 mmol) was dissolved in ACN (7 mL), and then NaOMe (0.15 g, 2.74 mmol) was added. The resulting reaction mixture was added to a mixture of di-tert-butyl dicarbonate and 1,2,3,5,6,7-hexahydro-s-indacen-4-amine, followed by stirring at 25° C. for 16 hours. The main peak of the desired mass was confirmed by LCMS. Water (20 mL) was poured into the mixture, extracted with EtOAc (20 mL×3), dried over Na₂SO₄, filtered, and concentrated under reduced pressure. The residue was purified by silica gel column chromatography (Hex:EtOAc=100:0 to 50:50) to obtain the title compound as a pale yellow solid (250 mg, 18% yield).

¹H NMR (400 MHz, DMSO-d₆) δ=11.12 (brs, 1H), 8.10 (s, 1H), 7.30-7.27 (m, 3H), 7.15-7.13 (m, 2H), 6.93 (s, 1H), 5.78 (s, 2H), 4.28 (q, J=6.8 Hz, 2H), 2.77 (t, J=7.2 Hz, 4H), 2.55 (t, J=6.8 Hz, 4H), 1.94-1.86 (m, 4H), 1.25 (t, J=7.2 Hz, 3H).

MS[M+H]=509.1

### Step 5. Synthesis of 1-benzyl-3-(N-((1,2,3,5,6,7-hexahydro-s-indacen-4-yl)carbamoyl)sulfamoyl)-1H-pyrazol-5-carboxylic acid (21)

To a solution of ethyl-1-benzyl-3-(N-((1,2,3,5,6,7-hexahydro-s-indacen-4-yl)carbamoyl)sulfamoyl)-1H-pyrazol-5-carboxylate (250 mg, 0.49 mmol) in THF/H₂O (1.31 mL:0.33 mL), LiOH·H₂O (25 mg, 0.60 mmol) in water (0.5 mL) was added and the resulting mixture was stirred at 40° C. for 3 hours. The main peak of the desired mass was confirmed by LCMS. The reaction mixture was concentrated in vacuo. To the residue, 1M HCl (10 mL) was added dropwise to precipitate a solid to obtain the title compound as a white solid (154 mg, 65% yield).

¹H NMR (600 MHz, DMSO-d₆) δ=8.15 (brs, 1H), 7.58 (s, 1H), 7.34-7.31 (m, 1H), 7.27-7.25 (m, 1H), 7.19-7.17 (m, 1H), 7.13-7.11 (m, 1H), 7.06 (s, 1H), 6.92 (s, 1H), 6.32 (s, 1H), 5.81 (d, J=19.7 Hz, 2H), 2.80-2.75 (m, 2H), 2.70-2.66 (m, 2H), 2.58-2.53 (m, 4H), 1.97-1.87 (m, 4H).

MS[M+H]=481.1

### Step 6. Synthesis of 1-benzyl-N-(2-(2-(2-((2-(2,6-dioxopiperidin-3-yl)-1,3-dioxoisoindolin-4-yl)amino)ethoxy)ethoxy)ethyl)-3-(N-((1,2,3,5,6,7-hexahydro-s-indacen-4-yl)carbamoyl)sulfamoyl)-1H-pyrazol-5-carboxamide (Compound 11)

To a solution of 1-benzyl-3-(N-((1,2,3,5,6,7-hexahydro-s-indacen-4-yl)carbamoyl)sulfamoyl)-1H-pyrazol-5-carboxylic acid (120 mg, 0.25 mmol) in DMF (1.5 mL), HATU (114 mg, 0.30 mmol), DIPEA (217 μL, 1.25 mmol) and 4-((2-(2-(2-aminoethoxy)ethoxy)ethyl)amino)-2-(2,6-dioxopiperidin-3-yl)isoindoline-1,3-dione (220 mg, 0.50 mmol) were added. The resulting mixture was stirred at room temperature for 2 hours. The main peak of the desired mass was confirmed by LCMS. Water (10 mL) was poured into the mixture, extracted with DCM (10 mL×3), dried over Na₂SO₄, and filtered. The residue was purified by silica gel column chromatography (DCM:MeOH=100:0 to 90:10) to obtain the yellow title compound (90 mg, 103.8 μmol, 41% yield, 93% purity).

¹H NMR (600 MHz, DMSO-d₆) δ=11.10 (s, 1H), 8.66 (brs, 1H), 7.59-7.55 (m, 2H), 7.27-7.24 (m, 5H), 7.14-7.12 (m, 1H), 7.03 (d, J=6.9 Hz, 1H), 6.79 (brs, 1H), 6.60 (t, J=6.0 Hz, 1H), 5.74 (s, 2H), 5.05 (dd, J=12.8, 5.5 Hz, 1H), 3.61-3.51 (m, 4H), 3.48-3.36 (m, 6H), 3.32-3.30 (m, 4H), 2.90-2.86 (m, 2H), 2.75-2.70 (m, 2H), 2.64-2.57 (m, 6H), 2.03-1.86 (m, 4H).

MS[M+H]=867.2

### <Example 12> Synthesis of 1-benzyl-N-(2-(2-(2-(2-((2-(2,6-dioxoisoindolin-4-yl)amino)ethoxy)ethoxy)ethoxy)ethyl)-3-(N-((1,2,3,5,6,7-hexahydro-s-indacen-4-yl)carbamoyl)sulfamoyl)-1H-pyrazol-5-carboxamide (Compound 12)

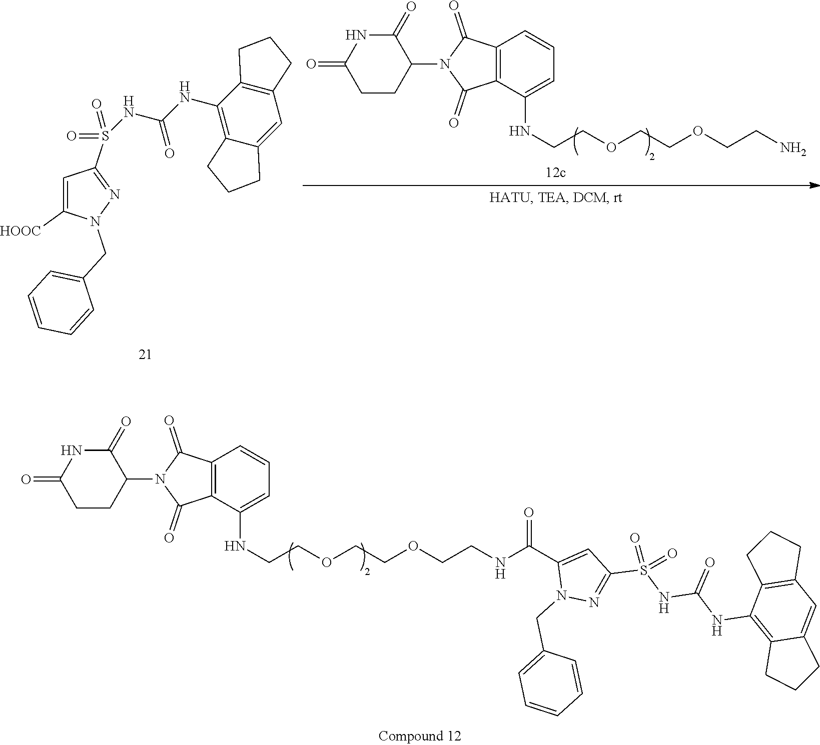

### Step 1. Synthesis of 1-benzyl-N-(2-(2-(2-(2-((2-(2,6-dioxoisoindolin-4-yl)amino)ethoxy)ethoxy)ethoxy)ethyl)-3-(N-((1,2,3,5,6,7-hexahydro-s-indacen-4-yl)carbamoyl)sulfamoyl)-1H-pyrazol-5-carboxamide (Compound 12)

To a solution of 1-benzyl-3-(N-((1,2,3,5,6,7-hexahydro-s-indacen-4-yl)carbamoyl)sulfamoyl)-1H-pyrazol-5-carboxylic acid (154 mg, 0.32 mmol) in DCM (2.6 mL), HATU (146 ma, 0.39 mmol), TEA (89 μL, 0.64 mmol) and 4-((2-(2-(2-aminoethoxy)ethoxy)ethoxy)ethyl)amino)-2-(2,6-dioxopiperidin-3-yl)isoindoline-1,3-dione (158 mg, 0.35 mmol) were added. The resulting mixture was stirred at room temperature for 2 hours. The main peak of the desired mass was confirmed by LCMS. Water (10 mL) was poured into the mixture, and the resulting mixture was extracted with DCM (10 mL×3), dried over Na₂SO₄, and filtered. The residue was purified by silica gel column chromatography (DCM:MeOH=100:0 to 90:10) to obtain the yellow title compound (2.5 mg, 2.7 μmol, 0.86% yield, 94% purity).

[M+H]=911.2

### Total Synthesis of Compound 13˜Compound 15

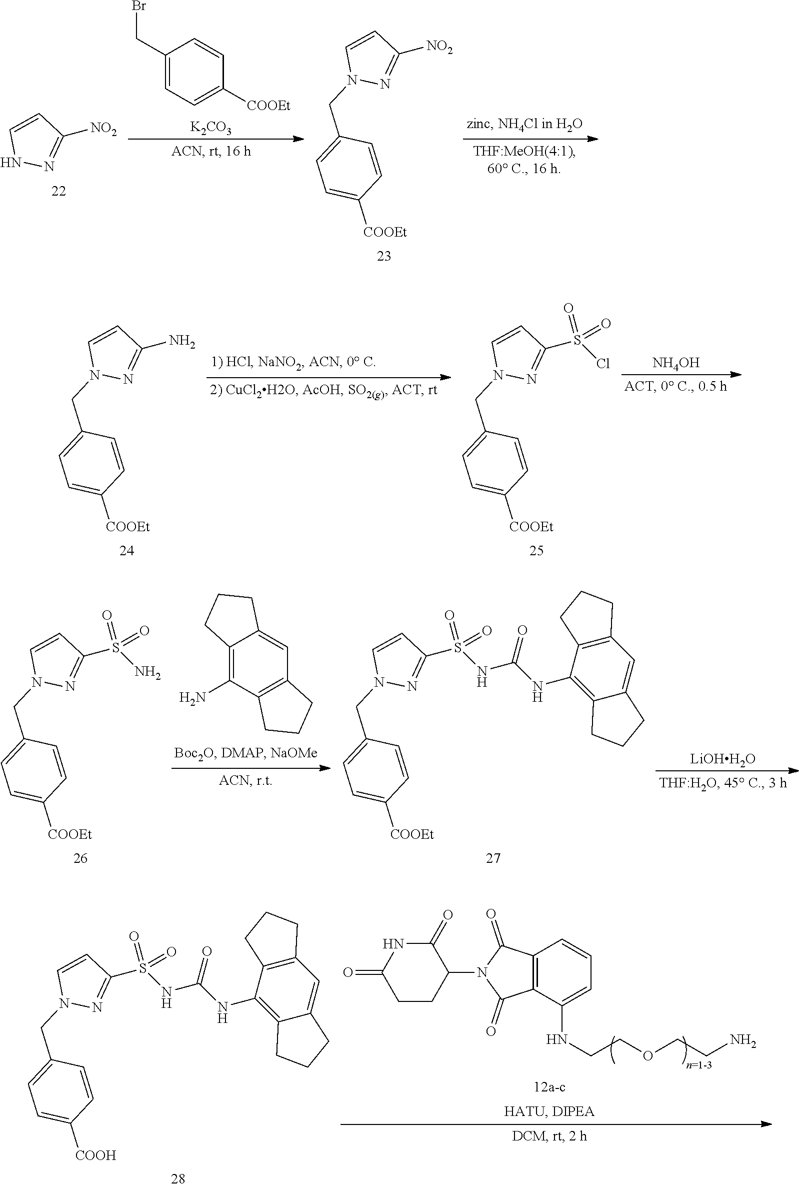

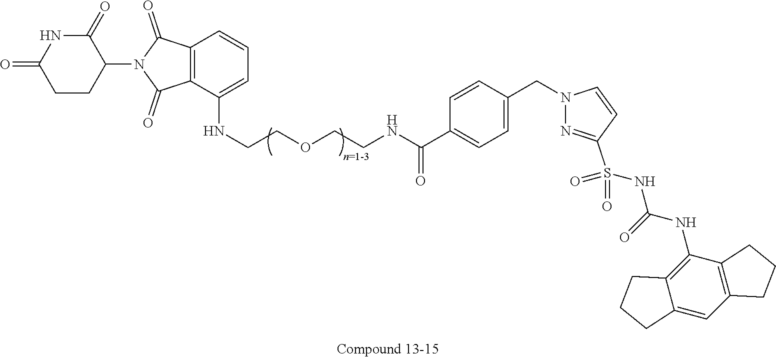

### <Example 13> Synthesis of N-(2-(2-((2-(2,6-dioxopiperidin-3-yl)-1,3-dioxoisoindolin-4-yl)amino)ethoxy)ethyl)-4-((3-(N-((1,2,3,5,6,7-hexahydro-s-indacen-4-yl)carbamoyl)sulfamoyl)-1H-pyrazol-1-yl)methyl)benzamide (Compound 13)

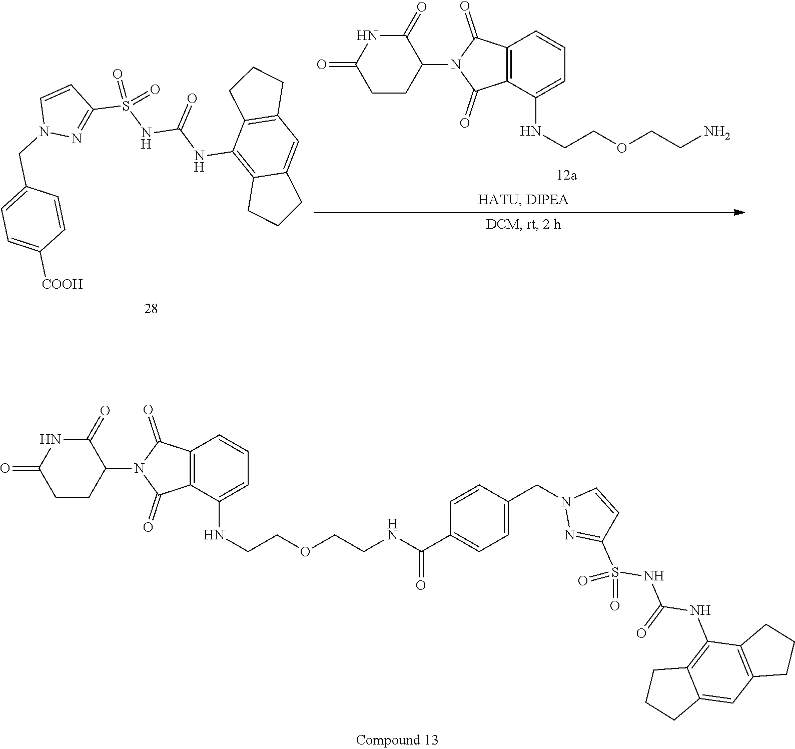

### Step 1. Synthesis of ethyl-4-((3-nitro-1H-pyrazol-1-yl)methyl)benzoate (23)

To a solution of 3-nitro-1H-pyrazole (1 g, 8.80 mmol) in ACN (29 mL), ethyl-4-(bromomethyl)benzoate (2.15 g, 8.80 mmol) and K₂CO₃ (1.83 g, 13.26 mmol) were added and stirred at 25° C. for 16 hours. The main peak of the desired mass was confirmed by LCMS. Water (100 mL) was poured into the mixture, extracted with EtOAc (20 mL×3), dried over Na₂SO₄, filtered, and concentrated under reduced pressure. The residue was purified by silica gel column chromatography (Hex:EtOAc=100:0 to 50:50) to obtain the title compound as a white solid (1.65 g, 68% yield).

MS[M+H]=276.1

### Step 2. Synthesis of ethyl-4-((3-amino-1H-pyrazol-1-yl)methyl)benzoate (24)

To a solution of ethyl-4-((3-nitro-1H-pyrazol-1-yl)methyl)benzoate (1.65 g, 5.99 mmol) in THF/MeOH (16 mL: 4 mL), Zinc (2.0 g, 29.96 mmol) was added. NH₄Cl (1.60 g, 29.96 mmol) in H₂O (4.5 mL) was added to the resulting mixture and stirred at 60° C. for 16 hours. The main peak of the desired mass was confirmed by LCMS. The reaction mixture was filtered with celite using EtOAc and concentrated under reduced pressure. The residue was purified by silica gel column chromatography (DCM:MeOH=100:0 to 90:10) to obtain the title compound as a yellow solid (0.98 g, 67% yield).

MS[M+H]=246.1

### Step 3. Synthesis of ethyl-4-((3-sulfamoyl-1H-pyrazol-1-yl)methyl)benzoate (26)

Ethyl-4-((3-amino-1H-pyrazol-1-yl)methyl)benzoate (1.5 g, 6.11 mmol) was placed in a two-necked flask and dissolved in ACN (61 mL). To the resulting mixture, conc HCl (13.5 mL) and NaNO₂ (0.51 g, 7.39 mmol) were added and stirred at 0° C. for 1 hour. After adding AcOH (13.5 mL) and CuCl₂·2H₂O (1.04 g, 6.11 mmol) to the reaction mixture, the mixture was stirred for 3 hours at 0° C. under SO₂ gas. The main peak of the desired intermediate mass was confirmed by LCMS. The reaction mixture was quenched by adding H₂O (500 mL) and extracted with DCM (200 mL×3). The extract was dried over Na₂SO₄, filtered, and concentrated under reduced pressure. The residue was dissolved in acetone (44 mL), then NH₄OH (22 mL) was added and stirred at 0° C. for 30 minutes. The main peak of the desired mass was confirmed by LCMS. After concentration of the reaction mixture in vacuo, water (50 mL) was poured into the residue, extracted with EtOAc (50 mL×3), dried over Na₂SO₄, filtered, and concentrated under reduced pressure. The residue was purified by silica gel column chromatography (Hex:EtOAc=50:50 to 20:80) to obtain the title compound as a white solid (278 mg, 15% yield).

MS[M+H]=310.0

### Step 4. Synthesis of ethyl-4-(93-(N-((1,2,3,5,6,7-hexahydro-s-indacen-4-yl)carbamoyl)sulfamoyl)-1H-pyrazol-1-yl)methyl)benzoate (27)

To a solution of di-tert-butyl dicarbonate (0.29 g, 1.26 mmol) in ACN (4.5 mL), DMAP (77 mg, 0.63 mmol) was added, and stirred at 25° C. for 30 minutes. To the resulting reaction, 1,2,3,5,6,7-hexahydro-s-indacen-4-amine (0.15 g, 0.90 mmol) was added. In another flask, ethyl-4-((3-amino-1H-pyrazol-1-yl)methyl)benzoate (0.28 g, 0.90 mmol) was dissolved in ACN (4.5 mL), and NaOMe (49 mg, 0.90 mmol) was added. The resulting reaction mixture was added to a mixture of di-tert-butyl dicarbonate and 1,2,3,5,6,7-hexahydro-s-indacen-4-amine, followed by stirring at 25° C. for 16 hours. The main peak of the desired mass was confirmed by LCMS. Water (20 mL) was poured into the mixture, extracted with EtOAc (20 mL×3), dried over Na₂SO₄, filtered, and concentrated under reduced pressure. The residue was purified by silica gel column chromatography (DCM:MeOH=100:0 to 90:10) to obtain the title compound as a white solid (230 mg, 51% yield).

¹H NMR (400 MHz, DMSO-d₆) δ 10.86 (s, 1H), 8.08 (s, 1H), 7.86 (d, J=8.4 Hz, 2H), 7.29 (d, J=8.4 Hz, 2H), 6.91 (s, 1H), 6.80 (s, 1H), 5.54 (s, 2H), 4.29 (q, J=7.2 Hz, 2H), 2.76 (t, J=7.6 Hz, 4H), 2.54-2.50 (m, 4H), 1.93-1.85 (m, 4H), 1.30 (t, J=7.2 Hz, 3H).

MS[M+H]=509.1

### Step 5. Synthesis of 4-((3-(N-((1,2,3,5,6,7-hexahydro-s-indacen-4-yl)carbamoyl)sulfamoyl)-1-H-pyrazol-1-yl)methyl)benzoic acid (28)

To a solution of ethyl-4-(93-(N-((1,2,3,5,6,7-hexahydro-s-indacen-4-yl)carbamoyl)sulfamoyl)-1H-pyrazol-1-yl)methyl)benzoate (233 mg, 0.49 mmol) in THF/H₂O (1.2 mL: 0.3 mL), LiOH·H₂O (23 mg, 0.59 mmol) in water (0.5 mL) was added and the resulting mixture was stirred at 45° C. for 3 hours. The main peak of the desired mass was confirmed by LCMS. The reaction mixture was concentrated in vacuo. To the residue, 1M HCl (10 mL) was added dropwise to precipitate a solid to obtain the title compound as a white solid (187 mg, 79% yield).

¹H NMR (400 MHz, DMSO-d₆) δ 13.06 (s, 1H), 8.04 (s, 1H), 7.92 (s, 1H), 7.86 (d, J=8.4 Hz, 2H), 7.28 (d, J=8.4 Hz, 2H), 6.89 (s, 1H), 6.75 (s, 1H), 2.75 (t, J=7.2 Hz, 4H), 2.53 (t, J=7.2 Hz, 4H), 1.93-1.88 (m, 4H).

MS[M+H]=481.1

### Step 6. Synthesis of N-(2-(2-((2-(2,6-dioxopiperidin-3-yl)-1,3-dioxoisoindolin-4-yl)amino)ethoxy)ethyl)-4-((3-(N-((1,2,3,5,6,7-hexahydro-s-indacen-4-yl)carbamoyl)sulfamoyl)-1H-pyrazol-1-yl)methyl)benzamide (Compound 13)

To a solution of 4-((3-(N-((1,2,3,5,6,7-hexahydro-s-indacen-4-yl)carbamoyl)sulfamoyl)-1-H-pyrazol-1-yl)methyl)benzoic acid (93 mg, 0.19 mmol) in DCM (1.5 mL), HATU (89 mg, 0.23 mmol), DIPEA (54 μL, 0.38 mmol) and 4-((2-(2-aminoethoxy)ethyl)amino)-2-(2,6-dioxopiperidin-3-yl)isoindoline-1,3-dione (84 mg, 0.20 mmol) were added. The resulting mixture was stirred at room temperature for 2 hours. The main peak of the desired mass was confirmed by LCMS. Water (10 mL) was poured into the mixture, extracted with DCM (10 mL×3), dried over Na₂SO₄, and filtered. The residue was purified by silica gel column chromatography (DCM:MeOH=100:0 to 90:10) to obtain the yellow title compound (3.9 mg, 4.7 μmol, 2.4% yield, 95% purity).

¹H NMR (400 MHz, DMSO-d₆) δ11.11 (s, 1H), 7.87-7.80 (m, 2H), 7.56 (t, J=8.0 Hz, 1H), 7.39 (d, J=5.6 Hz, 1H), 7.15 (d, J=8.8 Hz, 1H), 7.03 (d, J=6.8 Hz, 1H), 6.88 (s, 1H), 6.66 (t, J=5.3 Hz, 1H), 5.05 (dd, J=12.8, 5.6 Hz, 1H), 3.86 (s, 2H), 3.57-3.54 (m, 2H), 3.50-3.47 (m, 2H), 2.81-2.78 (m, 4H), 2.76-2.72 (m, 4H), 2.54-2.52 (m, 4H), 1.93-1.87 (m, 4H).

MS[M+H]=823.2

### <Example 14> Synthesis of N-(2-(2-(2-((2-(2,6-dioxopiperidin-3-yl)-1,3-dioxoisoindolin-4-yl)amino)ethoxy)ethoxy)ethyl-4-((3-(N-((1,2,3,5,6,7-hexahydro-s-indacen-4-yl)carbamoyl)sulfamoyl)-1H-pyrazol-1-yl)methyl)benzamide (Compound 14)

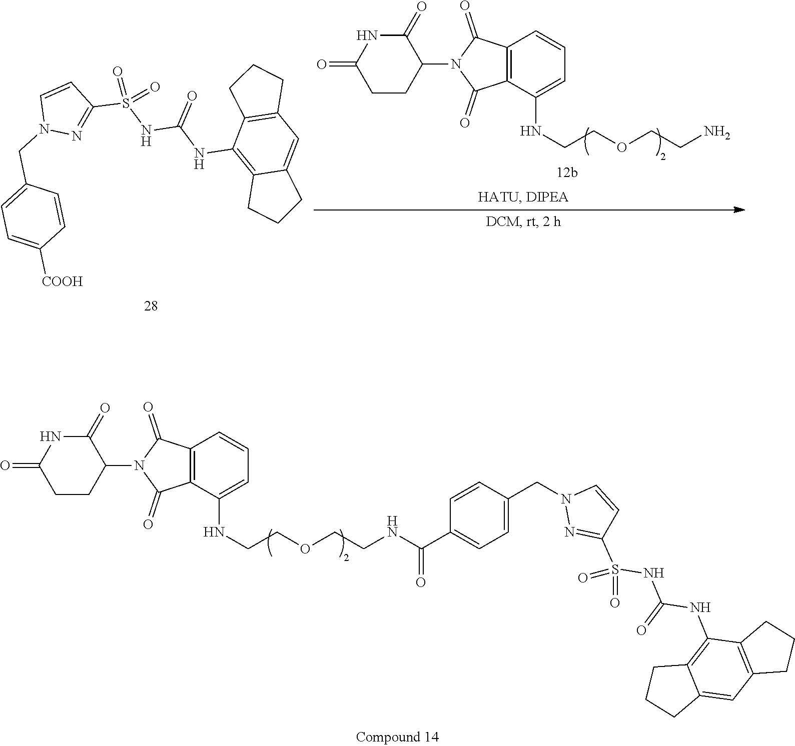

### Step 1. Synthesis of N-(2-(2-(2-((2-(2,6-dioxopiperidin-3-yl)-1,3-dioxoisoindolin-4-yl)amino)ethoxy)ethoxy)ethyl-4-((3-(N-((1,2,3,5,6,7-hexahydro-s-indacen-4-yl)carbamoyl)sulfamoyl)-1H-pyrazol-1-yl)methyl)benzamide (Compound 14)

To a solution of 4-((3-(N-((1,2,3,5,6,7-hexahydro-s-indacen-4-yl)carbamoyl)sulfamoyl)-1-H-pyrazol-1-yl)methyl)benzoic acid (93 mg, 0.19 mmol) in DCM (1.5 mL), HATU (89 mg, 0.23 mmol), DIPEA (54 μL, 0.38 mmol) and 4-((2-(2-(2-aminoethoxy)ethoxy)ethyl)amino)-2-(2,6-dioxopiperidin-3-yl)isoindoline-1,3-dione (86 mg, 0.20 mmol) were added. The resulting mixture was stirred at room temperature for 2 hours. The main peak of the desired mass was confirmed by LCMS. Water (10 mL) was poured into the mixture, extracted with DCM (10 mL×3), dried over Na₂SO₄, and filtered. The residue was purified by silica gel column chromatography (DCM:MeOH=100:0 to 90:10) to obtain the yellow title compound (9.4 mg, 10.8 μmol, 5.7% yield, 93% purity).

¹H NMR (400 MHz, DMSO-d₆) δ 11.09 (s, 1H), 8.46 (t, J=5.6 Hz, 1H), 8.04 (s, 1H), 7.94 (s, 1H), 7.76 (d, J=8.4 Hz, 2H), 7.58-7.54 (m, 1H), 7.26 (d, J=8.4 Hz, 2H), 7.10 (d, J=8.4 Hz, 1H), 7.02 (d, J=7.2 Hz, 1H), 6.89 (s, 1H), 6.77 (s, 1H), 6.59 (t, J=5.2 Hz, 1H), 5.48 (s, 2H), 3.61-3.50 (m, 8H), 3.43-3.38 (m, 4H), 2.75 (t, J=7.2 Hz, 2.52 (t, J=7.2 Hz, 4H), 1.91-1.85 (m, 4H) MS[M+H]=867.2

### <Example 15> Synthesis of N-(2-(2-(2-(2-((2-(2,6-dioxopiperidin-3-yl)-1,3-dioxoisoindolin-4-yl)amino)ethoxy)ethoxy)ethoxy)ethyl-4-((3-(N-((1,2,3,5,6,7-hexahydro-s-indacen-4-yl)carbamoyl)sulfamoyl)-1H-pyrazol-1-yl)methyl)benzamide (Compound 15)

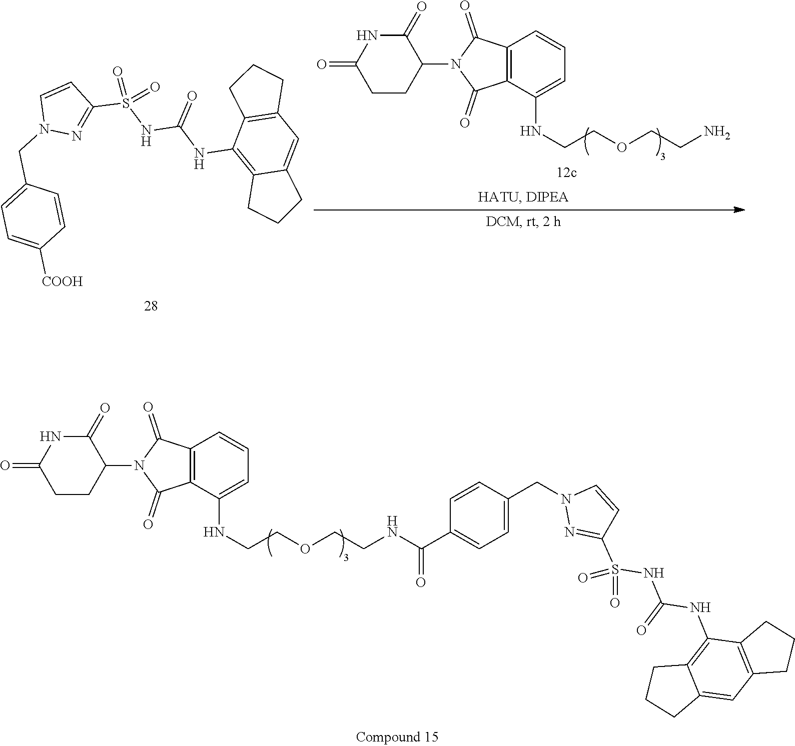

### Step 1. Synthesis of N-(2-(2-(2-(2-((2-(2,6-dioxopiperidin-3-yl)-1,3-dioxoisoindolin-4-yl)amino)ethoxy)ethoxy)ethoxy)ethyl-4-((3-(N-((1,2,3,5,6,7-hexahydro-s-indacen-4-yl)carbamoyl)sulfamoyl)-1H-pyrazol-1-yl)methyl)benzamide (Compound 15)

To a solution of 4-((3-(N-((1,2,3,5,6,7-hexahydro-s-indacen-4-yl)carbamoyl)sulfamoyl)-1-H-pyrazol-1-yl)methyl)benzoic acid (153 mg, 0.32 mmol) in DCM (2.5 mL), HATU (145 mg, 0.38 mmol), DIPEA (89 μL, 0.64 mmol), and 4-((2-(2-(2-(2-aminoethoxy)ethoxy)ethoxy)ethyl)amino)-2-(2,6-dioxopiperidin-3-yl)isoindoline-1,3-dione (157 mg, 0.35 mmol) were added. The resulting mixture was stirred at room temperature for 2 hours. The main peak of the desired mass was confirmed by LCMS. Water (10 mL) was poured into the mixture, extracted with DCM (10 mL×3), dried over Na₂SO₄, and filtered. The residue was purified by silica gel column chromatography (DCM:MeOH=100:0 to 90:10) to obtain the yellow title compound (8.1 mg, 8.8 μmol, 97% purity).

¹H NMR (400 MHz, DMSO-d₆) δ 11.09 (s, 1H), 8.46-8.45 (m, 1H), 7.76 (d, J=8.4 Hz, 2H), 7.57 (t, J=8.0 Hz, 1H), 7.26 (d, J=8.0 Hz, 2H), 7.12 (d, J=8.4 Hz, 1H), 7.03 (d, J=7.2 Hz, 1H), 6.82 (brs, 1H), 6.58 (t, J=5.2 Hz, 1H), 5.41 (s, 2H), 5.05 (dd, J=12.8, 5.2 Hz, 1H), 3.58-3.57 (m, 2H), 3.52-3.48 (m, 12H), 3.38-3.30 (m, 6H), 2.74 (t, J=7.2 Hz, 4H), 2.58-2.55 (m, 4H), 1.90-1.86 (m, 4H).

MS[M+H]=911.2

### Total Synthesis of Compound 16˜Compound 17

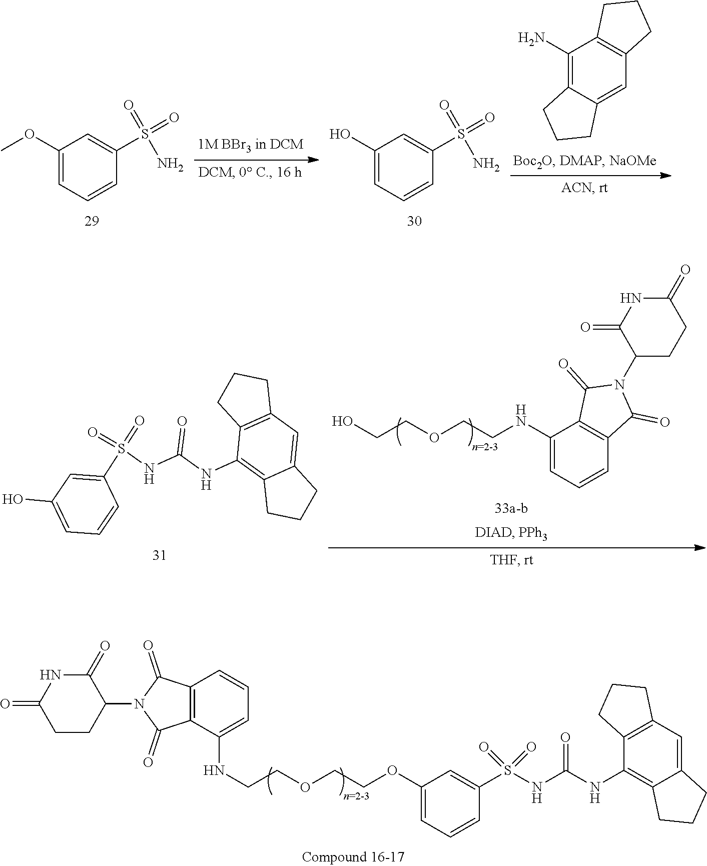

### <Example 16> Synthesis of 3-(2-(2-(2-((2-(2,6-dioxopiperidin-3-yl)-1,3-dioxoisoindolin-4-yl)amino)ethoxy)ethoxyethoxy)-N-((1,2,3,5,6,7-hexahydro-s-indacen-4-yl)carbamoyl)benzenesulfonamide (Compound 16)

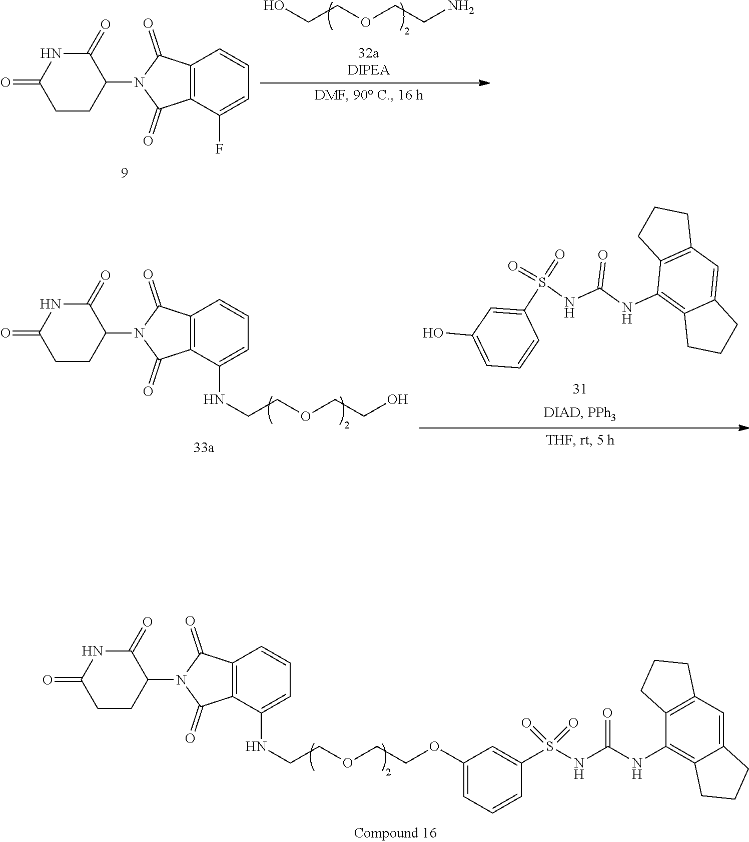

### Step 1. Synthesis of 3-hydroxybenzenesulfonamide (30)

To a solution of 3-methoxybenzenesulfonamide (1 g, 5.34 mmol) in DCM (100 mL), 1.0 M BBr₃ in DCM (27 mL, 27.2 mmol) was added dropwise at 0° C. The resulting mixture was stirred at room temperature for 16 hours. The main peak of the desired mass was confirmed by LCMS. The mixture was quenched by adding MeOH (100 mL) at 0° C. and extracted with EtOAc (100 mL×3). The organic phase was washed with H₂O (100 mL×3), dried over Na₂SO₄, filtered, and concentrated under reduced pressure. The title compound was obtained as a white solid (710 mg, 76%).

MS [M+H]=174.0

### Step 2. Synthesis of N-((1,2,3,5,6,7-hexahydro-s-indacen-4-yl)carbamoyl-3-hydroxybenzenesulfonamide (31)

To a solution of di-tert-butyl dicarbonate (1.3 mL, 5.74 mmol) in ACN (10 mL), DMAP (330 mg, 2.87 mmol) was added, and stirred at 25° C. for 30 minutes. To the resulting reaction mixture, 1,2,3,5,6,7-hexahydro-s-indacen-4-amine (0.71 g, 4.10 mmol) was added. In another flask, 3-hydroxybenzenesulfonamide (0.71 g, 4.10 mmol) was dissolved in ACN (10 mL), and then NaOMe (221 mg, 4.10 mmol) was added. The resulting reaction mixture was added to a mixture of di-tert-butyl dicarbonate and 1,2,3,5,6,7-hexahydro-s-indacen-4-amine, followed by stirring at 25° C. for 16 hours. The main peak of the desired mass was confirmed by LCMS. Water (50 mL) was poured into the mixture, extracted with EtOAc (50 mL×3), dried over Na₂SO₄, filtered, and concentrated under reduced pressure. The residue was purified by silica gel column chromatography (Hex:EtOAc=100:0 to 30:70) to obtain the title compound as a yellow solid (330 mg, 22% yield).

¹H NMR (400 MHz, DMSO-d₆) δ 10.75 (d, J=6.0 Hz, 1H), 8.08 (s, 1H), 7.43-7.32 (m, 3H), 7.06-7.03 (m, 1H), 6.93 (s, 1H), 2.76 (t, J=7.2 Hz, 4H), 2.54 (t, J=7.2 Hz, 4H), 1.93-1.90 (m, 4H).

MS[M+H]=373.7

### Step 3. Synthesis of 2-(2,6-dioxopiperidin-3-yl)-4-(92-(2-(2-hydroxyethoxy)ethoxy)ethyl)amino)isoindoline-1,3-dione (33a)

To a solution of 2(2,6-dioxopiperidin-3-yl)-4-fluoroisoindoline-1,3-dione (1 g, 3.60 mmol) and 2-(2-(2-aminoethoxy)ethoxy)ethan-1-ol (540 mg, 3.60 mmol) in DMF (20 mL), DIPEA (630 μL, 3.62 mmol) was added. The resulting mixture was stirred at 90° C. for 16 hours. Water (100 mL) was poured into the mixture, followed by extraction with EtOAc (100 mL×3). The organic phase was washed with H₂O (100 mL×3), dried over Na₂SO₄, filtered, and concentrated under reduced pressure. The residue was purified by silica gel column chromatography (DCM:MeOH=100:0 to 95:5) to obtain the title compound as a yellow liquid (200 mg, 14% yield).

### Step 4. Synthesis of 3-(2-(2-(2-((2-(2,6-dioxopiperidin-3-yl)-1,3-dioxoisoindolin-4-yl)amino)ethoxy)ethoxyethoxy)-N-((1,2,3,5,6,7-hexahydro-s-indacen-4-yl)carbamoyl)benzenesulfonamide (Compound 16)

To a solution of N-((1,2,3,5,6,7-hexahydro-s-indacen-4-yl)carbamoyl-3-hydroxybenzenesulfonamide (60 mg, 0.16 mmol), 2-(2,6-dioxopiperidin-3-yl)-4-((2-(2-(2-hydroxyethoxy)ethoxy)ethyl)amino)isoindoline-1,3-dione (65 mg, 0.16 mmol) in THF (2 mL), PPh₃ (63 mg, 0.24 mmol) was added. DIAD (48 μL, 0.24 mmol) was added to the resulting mixture at 0° C. The resulting mixture was stirred at room temperature for 5 hours. The main peak of the desired mass was confirmed by LCMS. Water (10 mL) was poured into the mixture, followed by extraction with EtOAc (10 mL×3). The organic phase was washed with H₂O (10 mL×3), dried over Na₂SO₄, filtered, and concentrated under reduced pressure. The residue was purified by silica gel column chromatography (EtOAc:MeOH=100:0 to 90:10) to obtain the title compound as a yellow solid (2 mg, 2.6 μmol, 1.6% yield).

¹H NMR (400 MHz, CDCl3) δ=8.70 (s, 1H), 8.07-8.05 (m, 1H), 7.87-7.85 (m, 2H), 7.55-7.49 (m, 1H), 7.12 (d, J=7.0 Hz, 1H), 7.00 (s, 1H), 6.93-6.88 (m, 3H), 4.94 (dd, J=12.1, 5.5 Hz, 1H), 3.99-3.97 (m, 2H), 3.76-3.72 (m, 4H), 3.43 (t, J=5.2 Hz, 3H), 2.92-2.66 (m, 14H), 2.07-2.00 (m, 5H).

MS[M+H]=760.2

### <Example 17> Synthesis of 3-(2-(2-(2-(2-((2-(2,6-dioxopiperidin-3-yl)-1,3-dioxoisoindolin-4-yl)amino)ethoxy)ethoxy)ethoxy)ethoxy)-N-((1,2,3,5,6,7-hexahydro-s-indacen-4-yl)carbamoyl)benzenesulfonamide (Compound 17)

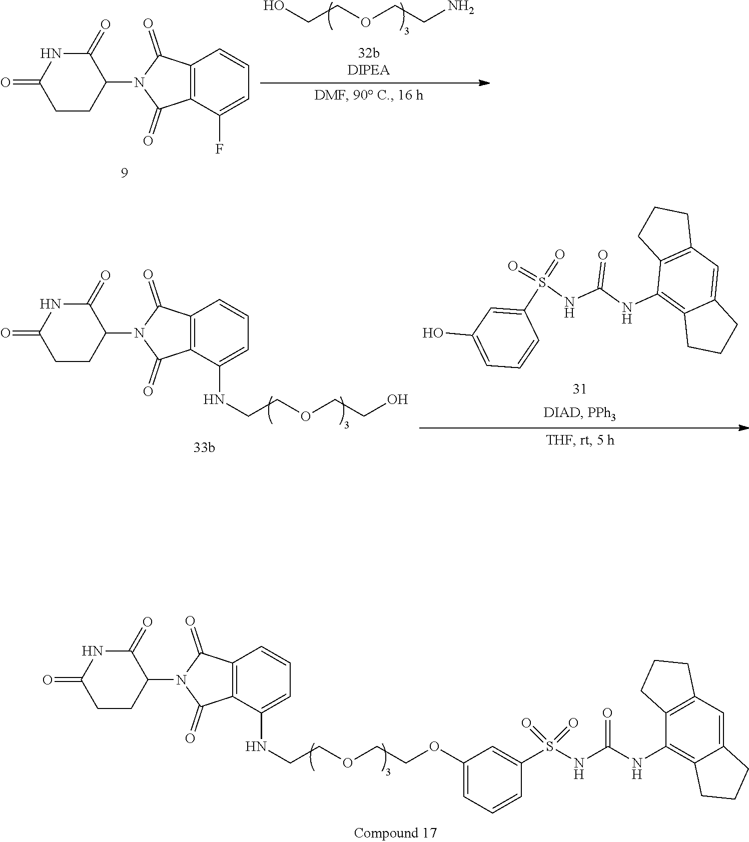

### Step 1. Synthesis of 2-(2,6-dioxopiperidin-3-yl)-4-((2-(2-(2-(2-hydroxyethoxy)ethoxy)ethoxy)ethyl)amino)isoindoline-1,3-dione (33b)

The title compound (500 mg, 31% yield) was obtained as a yellow solid in a similar manner to Step 3 of the synthesis scheme of Compound 12.

MS[M+H]=450.1

### Step 2. Synthesis of 3-(2-(2-(2-(2-((2-(2,6-dioxopiperidin-3-yl)-1,3-dioxoisoindolin-4-yl)amino)ethoxy)ethoxy)ethoxy)ethoxy)-N-((1,2,3,5,6,7-hexahydro-s-indacen-4-yl)carbamoyl)benzenesulfonamide (Compound 17)

To a solution of N-((1,2,3,5,6,7-hexahydro-s-indacen-4-yl)carbamoyl-3-hydroxybenzenesulfonamide (100 mg, 0.27 mmol), 2-(2,6-dioxopiperidin-3-yl)-4-((2-(2-(2-(2-hydroxyethoxy)ethoxy)ethoxy)ethyl)amino)isoindoline-1,3-dione (120 mg, 0.27 mmol) in THF (1 mL), PPh₃ (105 mg, 0.40 mmol) was added. DIAD (79 μL, 0.40 mmol) was added to the resulting mixture at 0° C. The resulting mixture was stirred at room temperature for 5 hours. The main peak of the desired mass was confirmed by LCMS. Water (10 mL) was poured into the mixture, followed by extraction with EtOAc (10 mL×3). The organic phase was washed with H₂O (10 mL×3), dried over Na₂SO₄, filtered, and concentrated under reduced pressure. The residue was purified by silica gel column chromatography (EtOAc:MeOH=100:0 to 90:10) to obtain the title compound as a yellow solid (54 mg, 67 μmol, 26% yield).

¹H NMR (400 MHz, DMSO-d₆) δ=11.11 (brs, 1H), 10.10 (brs, 1H), 7.66-7.64 (m, 1H), 7.58 (dd, J=8.6, 7.1 Hz, 1H), 7.37 (t, J=7.9 Hz, 1H), 7.21-7.14 (m, 3H), 7.04 (d, J=7.0 Hz, 1H), 7.01-6.98 (m, 1H), 6.97 (s, 1H), 6.60 (t, J=5.8 Hz, 1H), 5.05 (dd, J=12.9, 5.3 Hz, 1H), 3.63-3.38 (m, 18H), 2.87-2.80 (m, 10H), 2.08-2.00 (m, 4H).

MS[M+H]=804.2

### <Example 18> Synthesis of (2S,4R)-1-((S)-2-(tert-butyl)-14-(4-(N-((1,2,3,5,6,7-hexahydro-s-indacen-4)-yl)carbamoyl)sulfamoyl) phenoxy)-4-oxo-6,8,12-trioxa-3-azatetradecanoyl)-4-hydroxy-N-(4-(4-methylthiazol-5-yl)benzyl)pyrrolidine-2-carboxamide (Compound 18)

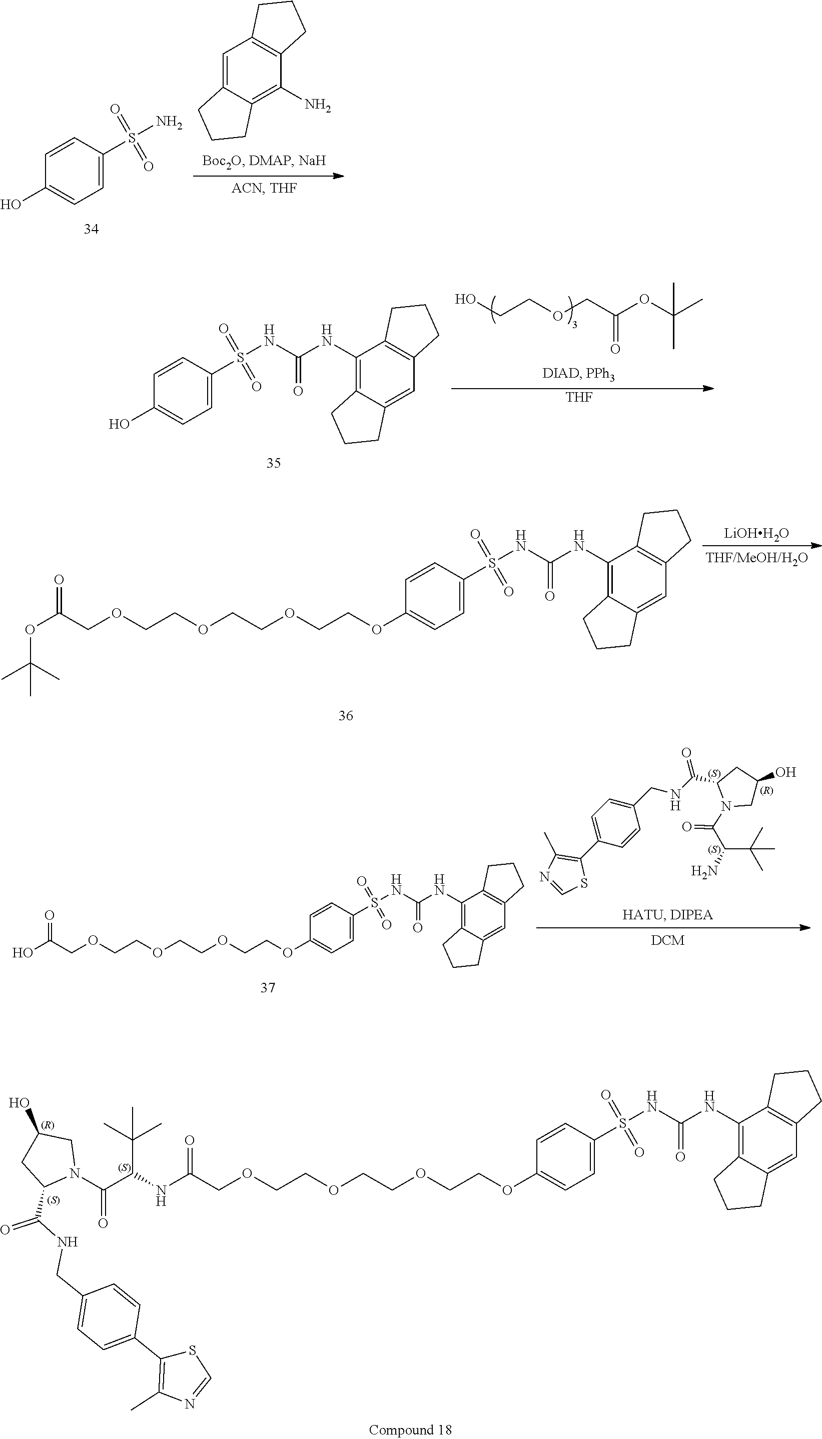

### Step 1. Synthesis of N-((1,2,3,5,6,7-hexahydro-s-indacen-4-yl)carbamoyl-4-hydroxybenzenesulfonamide (35)

To a solution of di-tert-butyl dicarbonate (10.6 g, 48.48 mmol) in ACN (60 mL), DMAP (2.12 g, 17.32 mmol) was added, and stirred at 25° C. for 30 minutes. To the resulting reaction mixture, 1,2,3,5,6,7-hexahydro-s-indacen-4-amine (6.0 g, 34.63 mmol) was added. In another flask, 4-hydroxybenzenesulfonamide (6.0 g, 34.63 mmol) was dissolved in ACN (60 mL), then NaH (1.39 g, 34.63 mmol) was added under nitrogen gas at 0° C., and stirred at room temperature for 30 minutes. The resulting reaction mixture was added to a mixture of di-tert-butyl dicarbonate and 1,2,3,5,6,7-hexahydro-s-indacen-4-amine, followed by stirring at 25° C. for 16 hours. The main peak of the desired mass was confirmed by LCMS. The reaction mixture was filtered and washed with ACN (30 mL). Water was added to the solid and acidified to pH 5 with 1N HCl solution. The mixture was stirred until a suspension occurred, and the solid was dried for 5 hours after filtration under reduced pressure. The solid was washed with EtOAc (30 mL), and dried for 18 hours after filtration under reduced pressure. The title compound was obtained as a white solid (6.92 g, 54% yield).

MS[M+H]=373.1

### Step 2. Synthesis of tert-butyl-2-(2-(2-(2-(4-(N-((1,2,3,5,6,7-hexahydro-s-indacen-4-yl)carbamoyl)sulfamoyl)phenoxy)ethoxy)ethoxy)ethoxy)acetate (36)

To a solution of N-((1,2,3,5,6,7-hexahydro-s-indacen-4-yl)carbamoyl)-4-hydroxybenzenesulfonamide (500 mg, 1.34 mmol) in THF (10 mL), tert-butyl-2-(2-(2-(2-hydroxyethoxy)ethoxy)ethoxy)acetate (355 mg, 1.34 mmol), PPh₃ (5.28 mg, 2.01 mmol) and DIAD (0.395 mL, 2.01 mmol) were added. The resulting mixture was stirred at room temperature for 20 hours. The main peak of the desired mass was confirmed by LCMS. Water (30 mL) was poured into the mixture, followed by extraction with EtOAc (10 mL×3). The organic phase was washed with brine (10 mL×3), dried over Na₂SO₄, filtered, and concentrated under reduced pressure. The residue was purified by silica gel column chromatography (Hex:EtOAc=50:50 to 30:70→DCM:MeOH=97:3) to obtain the title compound as a clear liquid (108 mg, 13% yield).

MS[M+Na]=641.2

### Step 3. Synthesis of 2-(2-(2-(2-(4-(N-((1,2,3,5,6,7-hexahydro-s-indacen-4-yl)carbamoyl)sulfamoyl)phenoxy)ethoxy)ethoxy)ethoxy)acetic acid (37)

To a solution of tert-butyl-2-(2-(2-(2-(4-(N-((1,2,3,5,6,7-hexahydro-s-indacen-4-yl)carbamoyl)sulfamoyl)phenoxy)ethoxy)ethoxy)ethoxy)acetate (100 mg, 0.162 mmol) in THF:MeOH (0.8 mL:0.2 mL), lithium hydroxide monohydrate (8.00 mg, 0.178 mmol) in water (0.2 mL) was added and stirred at room temperature for 24 hours. The main peak of the desired mass was confirmed by LCMS. Ethyl ether (10 mL) and water (10 mL) were added and layers were separated. The pH of the aqueous phase was acidified to pH 4-5 with 1N HCl solution and then extracted with EtOAc (10 mL×2). The organic phase was washed with brine (10 mL), dried over Na₂SO₄, filtered, and concentrated under reduced pressure. The title compound was obtained as a white solid (81 mg, 87% yield).

MS[M+H]=563.2

### Step 4. Synthesis of (2S,4R)-1-((S)-2-(tert-butyl)-14-(4-(N-((1,2,3,5,6,7-hexahydro-s-indacen-4)-yl)carbamoyl)sulfamoyl)phenoxy)-4-oxo-6,8,12-trioxa-3-azatetradecanoyl)-4-hydroxy-N-(4-(4-methylthiazol-5-yl)benzyl)pyrrolidine-2-carboxamide (Compound 18)

To a solution of 2-(2-(2-(2-(4-(N-((1,2,3,5,6,7-hexahydro-s-indacen-4-yl)carbamoyl)sulfamoyl)phenoxy)ethoxy)ethoxy)ethoxy)acetic acid (81 mg, 0.144 mmol) in DCM (2.0 mL), HATU (85 mg, 0.22 mmol), DIPEA (103 μL, 0.72 mmol) and (2S,4R)-1-((S)-2-amino-3,3-dimethylbutanoyl)-4-hydroxy-N-(4-(4-methylthiazol)-5-yl)benzyl)pyrrolidine-2-carboxamide (65 mg, 0.151 mmol) were added. The resulting mixture was stirred at room temperature for 3 hours. The main peak of the desired mass was confirmed by LCMS. Water (1 mL) was poured into the mixture, extracted with DCM (2 mL×2), dried over Na₂SO₄, and filtered. The residue was purified by silica gel column chromatography (DCM:MeOH=97:3 to 95:5) to obtain the white title compound (52.1 mg, 53.4 μmol, 57% yield, 92% purity).

¹H NMR (600 MHz, DMSO-d₆) δ 10.33 (s, 1H), 8.96 (s, 1H), 8.88 (s, 1H), 8.58 (s, 1H), 7.80-7.75 (m, 2H), 7.41-7.38 (m, 4H), 6.98 (s, 1H), 6.90-6.86 (m, 2H), 5.15 (d, J=3.6 Hz, 1H), 4.56 (d, J=9.6 Hz, 1H), 4.46-4.41 (m, 2H), 4.41-4.37 (m, 1H), 4.35 (s, 1H), 4.25 (dd, J=15.8, 5.7 Hz, 1H), 4.18 (dd, J=6.1, 3.1 Hz, 2H), 3.93 (s, 2H), 3.68-3.64 (m, 1H), 3.60 (d, J=10.8 Hz, 1H), 3.57-3.52 (m, 2H), 3.50-3.43 (m, 4H), 3.42-3.36 (m, 4H), 2.82-2.77 (m, 4H), 2.53 (t, J=7.3 Hz, 4H), 2.45-2.42 (m, 3H), 2.08-2.03 (m, 2H), 1.95-1.87 (m, 5H), 0.96-0.89 (m, 9H).

MS[M+H]=975.25

### <Example 19> Synthesis of 4-((10-((2-(2,6-dioxopiperidin-3-yl)-1,3-dioxoisoindolin-4-yl)amino) decyl)oxy)-N-((1,2,3,5,6,7-hexahydro-s-indacen-4-yl)carbamoyl)benzenesulfonamide (Compound 19)

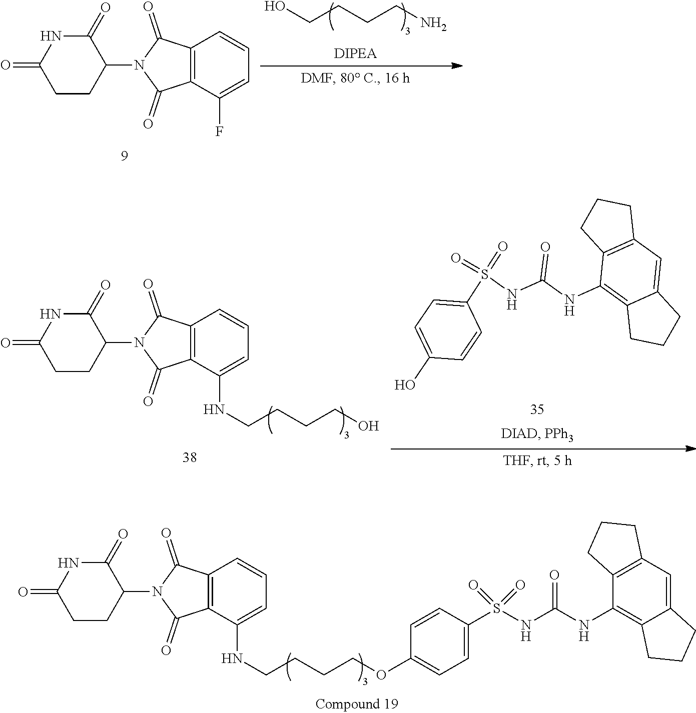

### Step 1. Synthesis of 2-(2,6-dioxopiperidin-3-yl)-4-((10-hydroxydecyl)amino)isoindoline-1,3-dione (38)

To a solution of 2(2,6-dioxopiperidin-3-yl)-4-fluoroisoindoline-1,3-dione (1 g, 3.60 mmol) and 10-aminodecane-lol (624 mg, 3.60 mmol) in DMF (20 mL), DIPEA (630 μL, 3.62 mmol) was added. The resulting mixture was stirred at 80° C. for 16 hours. The main peak of the desired mass was confirmed by LCMS. Water (100 mL) was poured into the mixture, followed by extraction with EtOAc (100 mL×3). The organic phase was washed with H₂O (100 mL×3), dried over Na₂SO₄, filtered, and concentrated under reduced pressure. The residue was purified by silica gel column chromatography (Hex:EtOAc=100:0 to 40:60) to obtain the title compound as a yellow liquid (200 mg, 14% yield).

MS[M+H]=429.4

### Step 2. Synthesis of 4-((10-((2-(2,6-dioxopiperidin-3-yl)-1,3-dioxoisoindolin-4-yl)amino)decyl)oxy)-N-((1,2,3,5,6,7-hexahydro-s-indacen-4-yl)carbamoyl)benzenesulfonamide (Compound 19)

To a solution of N-((1,2,3,5,6,7-hexahydro-s-indacen-4-yl)carbamoyl-3-hydroxybenzenesulfonamide (100 mg, 0.27 mmol), 2-(2,6-dioxopiperidin-3-yl)-4-((10-hydroxydecyl)amino)isoindoline-1,3-dione (115 mg, 0.27 mmol) in THF (1 mL), PPh₃ (105 mg, 0.40 mmol) was added. DIAD (79 μL, 0.40 mmol) was added to the resulting mixture at 0° C. The resulting mixture was stirred at room temperature for 5 hours. The main peak of the desired mass was confirmed by LCMS. Water (10 mL) was poured into the mixture, followed by extraction with EtOAc (10 mL×3). The organic phase was washed with H₂O (10 mL×3), dried over Na₂SO₄, filtered, and concentrated under reduced pressure. The residue was purified by silica gel column chromatography (EtOAc:MeOH=100:0 to 90:10) to obtain the title compound as a yellow solid (35 mg, 44 μmol, 16% yield).

¹H NMR (400 MHz, DMSO-d₆) δ=11.10 (s, 1H), 9.03 (s, 1H), 7.77-7.73 (m, 2H), 7.59-7.55 (m, 1H), 7.10-7.07 (m, 1H), 7.02-7.00 (m, 1H), 6.97 (s, 1H), 6.93-6.86 (m, 2H), 6.52 (t, J=5.9 Hz, 1H), 5.04 (dd, J=13.0, 5.2 Hz, 1H), 3.55 (t, J=7.0 Hz, 1H), 3.29-3.27 (m, 2H), 2.87-2.77 (m, 4H), 2.60-2.56 (m, 4H), 1.99-1.90 (m, 4H), 1.58-1.51 (m, 4H), 1.30-1.15 (m, 16H).

MS[M+H]=784.2

### <Example 20> Synthesis of 2-(2-((2-(2,6-dioxopiperidin-3-yl)-1,3-dioxoisoindolin-4-yl)amino)ethoxy)-N-(4 (N-((1,2,3,5,6,7-hexahydro-s-indacen-4-yl)carbamoyl)sulfamoyl) phenethyl) acetamide (Compound 20)

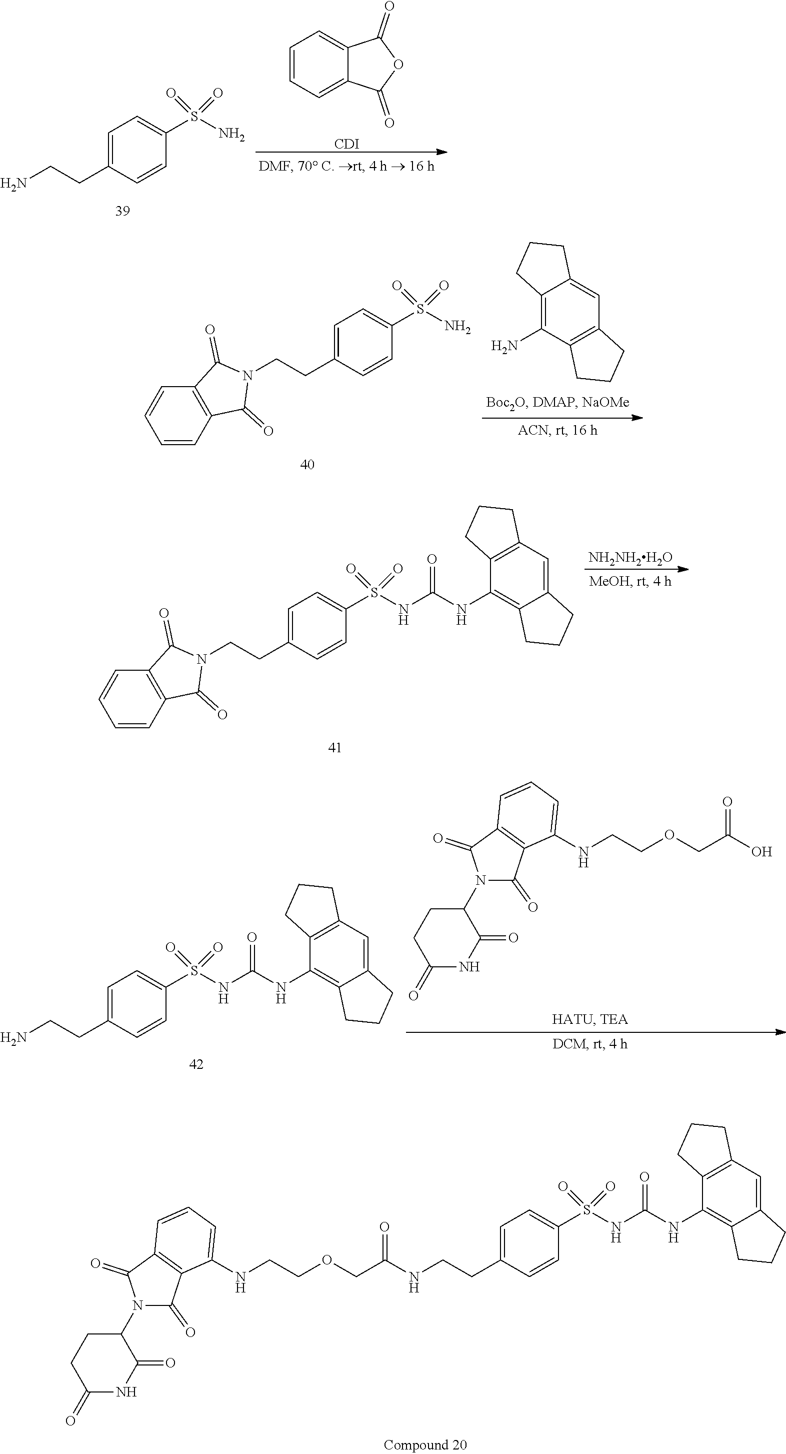

### Step 1. Synthesis of 4-(2-(1,3-dioxoisoindolin-2-yl)ethyl)benzenesulfonamide (40)

To a solution of 4-(2-aminoethyl)benzenesulfonamide (1 g, 4.99 mmol) in DMF (6 mL), phthalic anhydride (1.48 g, 9.98 mmol) was added. The product was stirred at 70° C. for 4 hours. After stirring at room temperature for 30 minutes, CDI (0.81 g, 4.99 mmol) was added and stirred at room temperature for 16 hours. Water (100 mL) was added and the formed solid was filtered under reduced pressure. The solid was washed with acetone (20 mL×3) and then washed with DCM (20 mL×3) to obtain the title compound as a white solid (1.79 g, quant).

MS[M+H]=331.0

### Step 2. Synthesis of 4-(2-(1,3-dioxoisoindolin-2-yl)ethyl)-N-((1,2,3,5,6,7-hexahydro-s-indacen-4-yl)carbamoyl)benzenesulfonamide (41)

To a solution of di-tert-butyl dicarbonate (1.74 mL, 7.59 mmol) in ACN (14 mL), DMAP (463 mg, 3.79 mmol) was added, and stirred at 25° C. for 30 minutes. To the resulting reaction mixture, 1,2,3,5,6,7-hexahydro-s-indacen-4-amine (0.94 g, 5.42 mmol) was added. 4-(2-(1,3-dioxoisoindolin-2-yl)ethyl)benzenesulfonamide (1.79 g, 5.42 mmol) was dissolved in ACN (14 mL), and then NaOMe (293 mg, 5.42 mmol) was added. The resulting reaction mixture was added to a mixture of di-tert-butyl dicarbonate and 1,2,3,5,6,7-hexahydro-s-indacen-4-amine, followed by stirring at 25° C. for 16 hours. The main peak of the desired mass was confirmed by LCMS. Water (50 mL) was poured into the mixture, extracted with EtOAc (50 mL×3), dried over Na₂SO₄, filtered, and concentrated under reduced pressure. The residue was purified by silica gel column chromatography (Hex:EtOAc=100:0 to 50:50) to obtain the title compound as a yellow solid (477 mg, 16% yield).

MS[M+H]=530.1

### Step 3. Synthesis of 4-(2-aminoethyl)-N-((1,2,3,5,6,7-hexahydro-s-indacen-4-yl)carbamoyl)benzenesulfonamide (42)

To a solution of 4-(2-(1,3-dioxoisoindolin-2-yl)ethyl)-N-((1,2,3,5,6,7-hexahydro-s-indacen-4-yl)carbamoyl)benzenesulfonamide (0.1 g, 0.19 mmol) in MeOH (2 mL), NH₂NH₂·H₂O (18 μL, 0.38 mmol) was added at 0° C. The resulting mixture was stirred at room temperature for 4 hours. The main peak of the desired mass was confirmed by LCMS and the mixture was concentrated under reduced pressure. Water (20 mL) was poured into the residue to precipitate a solid to obtain the title compound as a white solid (48.3 mg, 64% yield).

¹H NMR (400 MHz, DMSO-d₆) δ 7.70 (d, J=8.4 Hz, 2H), 7.50 (s, 2H), 7.19 (d, J=8.0 Hz, 2H), 6.76 (s, 1H), 2.95-2.91 (m, 2H), 2.81-2.77 (m, 2H), 2.73 (t, 7.2 Hz, 4H), 2.63 (t, J=7.2 Hz, 4H), 1.90-1.87 (m, 4H)

MS[M+H]=400.1

### Step 4. Synthesis of 2-(2-((2-(2,6-dioxopiperidin-3-yl)-1,3-dioxoisoindolin-4-yl)amino)ethoxy)-N-(4 (N-((1,2,3,5,6,7-hexahydro-s-indacen-4-yl)carbamoyl)sulfamoyl)phenethyl)acetamide (Compound 20)

To a solution of 4-(2-aminoethyl)-N-((1,2,3,5,6,7-hexahydro-s-indacen-4-yl)carbamoyl)benzenesulfonamide (129 mg, 0.32 mmol) in DCM (2.6 mL), HATU (148 mg, 0.39 mmol), TEA (90 μL, 0.64 mmol) and 4-((2-(2-aminoethoxy)ethyl)amino)-2-(2,6-dioxopiperidin-3-yl)isoindoline-1,3-dione (133 mg, 0.36 mmol) were added. The resulting mixture was stirred at room temperature for 4 hours. The main peak of the desired mass was confirmed by LCMS. Water (10 mL) was poured into the mixture, extracted with DCM (10 mL×3), dried over Na₂SO₄, and filtered. The residue was purified by silica gel column chromatography (DCM:MeOH=100:0 to 90:10) to obtain the yellow title compound (12 mg, 15.8 μmol, 91% purity).

¹H NMR (400 MHz, DMSO-d₆) δ 11.11 (s, 1H), 7.87-7.80 (m, 2H), 7.56 (t, J=8.0 Hz, 1H), 7.39-7.38 (m, 1H), 7.15 (d, J=8.8 Hz, 1H), 7.03 (d, J=6.8 Hz, 1H), 6.88 (s, 1H), 6.66 (t, J=5.6 Hz, 1H), 5.05 (dd, J=12.8, 5.6 Hz, 1H), 3.86 (s, 2H), 3.57-3.54 (m, 2H), 3.50-3.47 (m, 2H), 2.82-2.78 (m, 4H), 2.76-2.72 (m, 4H), 2.02-1.98 (m, 4H), 1.93-1.87 (m, 4H).

MS[M+H]=757.2

### <Example 21> Synthesis of 2-(2-(2-((2-(2,6-dioxopiperidin-3-yl)-1,3-dioxoisoindolin-4-yl)amino)ethoxy)ethoxy)-N-(3-(N-(1,2,3,5,6,7-hexahydro-s-indacen-4-yl)carbamoyl)sulfamoyl)phenyl) acetamide (Compound 21)

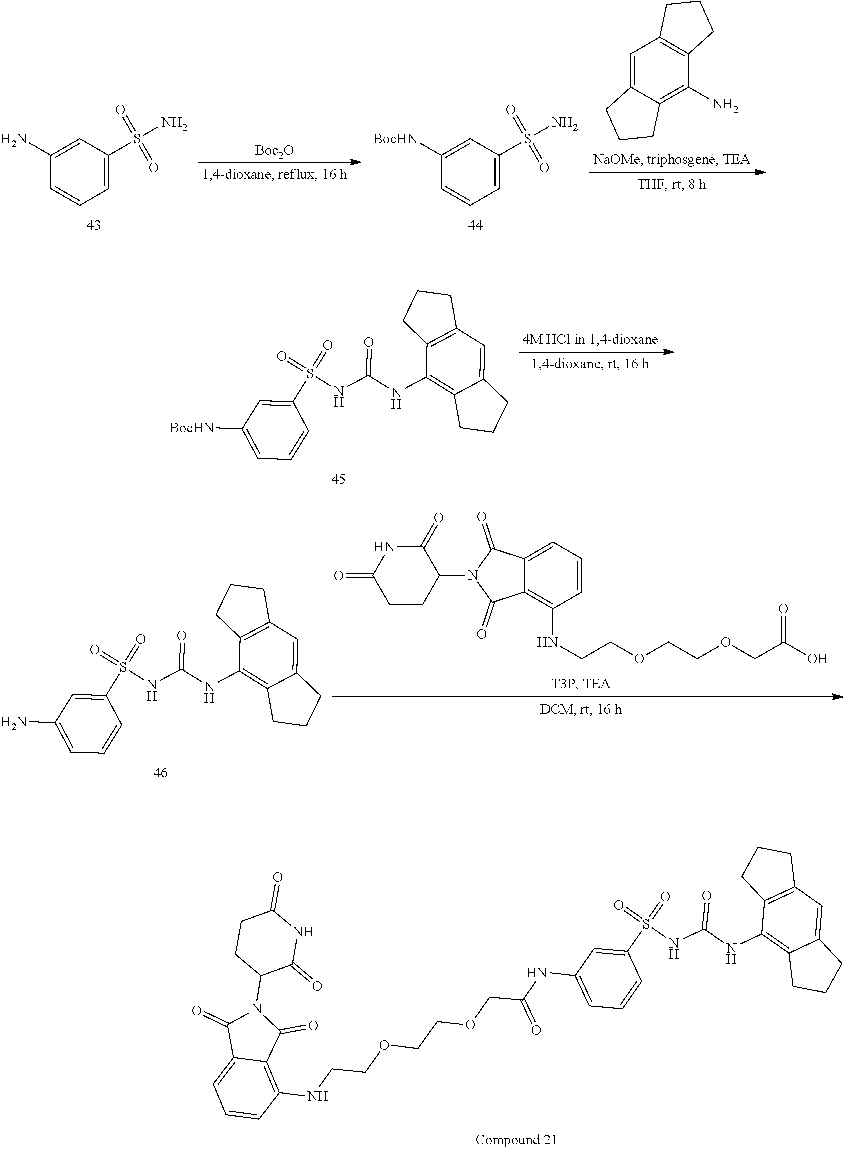

### Step 1. Synthesis of tert-butyl(3-sulfamoylphenyl) carbamate (44)

To a solution of 3-aminobenzenesulfonamide (2.0 g, 11.61 mmol) in dioxane (40 mL), Boc₂O (3.04 g, 13.94 mmol) was added dropwise. The product was stirred at 110° C. for 16 hours. The reaction mixture was concentrated under reduced pressure. Water (100 mL) was poured into the residue, extracted with EtOAc (100 mL×3), dried over Na₂SO₄, and filtered. The residue was purified by silica gel column chromatography (Hex:EtOAc=100:0 to 70:30) to obtain the white title compound (3.10 g, 98% yield).

### Step 2. Synthesis of tert-butyl (3-(N((1,2,3,5,6,7-hexahydro-s-indacen-4-yl)carbamoyl)sulfamoyl)phenyl)carbamate (45)

To a solution of tert-butyl(3-sulfamoylphenyl)carbamate (1.08 g, 3.96 mmol) in THF (20 mL), NaOMe (257 mg (4.76 mmol) was added and stirred at room temperature for 1 hour. Under nitrogen gas in another flask, 1,2,3,5,6,7-hexahydro-s-indacen-4-amine (1.03 g, 5.95 mmol) and TEA (1.26 mL, 9.12 mmol) were dissolved in THF (20 mL), and triphosgene (588 mg, 1.98 mmol) was added, followed by stirring for 30 minutes. The mixture of tert-butyl(3-sulfamoylphenyl)carbamate was added to the mixture of tert-butyl(3-sulfamoylphenyl)carbamate, and stirred at 25° C. for 8 hours. The reaction mixture was concentrated under reduced pressure. Water (100 mL) was poured into the residue, extracted with EtOAc (100 mL×3), dried over Na₂SO₄, and filtered. The residue was purified by silica gel column chromatography (Hex:EtOAc=100:0 to 70:30) to obtain the white title compound (685 mg, 36.6% yield).

### Step 3. Synthesis of 3-amino-N-((1,2,3,5,6,7-hexahydro-s-indacen-4-yl)carbamoyl)benzenesulfonamide (46)

To a solution of tert-butyl (3-(N((1,2,3,5,6,7-hexahydro-s-indacen-4-yl)carbamoyl)sulfamoyl)phenyl)carbamate (2 g, 4.24 mmol) in dioxane (5 mL), 4M HCl/dioxane (4M, 5 mL) was added. The resulting mixture was stirred at room temperature for 16 hours. The main peak of the desired mass was confirmed by LCMS. The reaction mixture was concentrated under reduced pressure to obtain the title compound as an ivory solid (1.2 g, 76% yield).

MS[M+H]=372.1

### Step 4. Synthesis of 2-(2-(2-((2-(2,6-dioxopiperidin-3-yl)-1,3-dioxoisoindolin-4-yl)amino)ethoxy)ethoxy)-N-(3-(N-(1,2,3,5,6,7-hexahydro-s-indacen-4-yl)carbamoyl)sulfamoyl)phenyl)acetamide (Compound 21)

To a solution of 3-amino-N-((1,2,3,5,6,7-hexahydro-s-indacen-4-yl)carbamoyl)benzenesulfonamide (50 mg, 0.13 mmol) in DCM (6.7 mL), T3P (0.1 mL, 0.13 mmol), TEA (37 μL, 0.27 mmol) and 2-(2-(2-((2-(2,6-dioxopiperidin-3-yl)-1,3-dioxoisoindolin-4-yl)amino)ethoxy)ethoxy)acetic acid (56 mg, 0.13 mmol) were added. The resulting mixture was stirred at room temperature for 16 hours. The main peak of the desired mass was confirmed by LCMS. Water (10 mL) was poured into the mixture, extracted with DCM (10 mL×3), dried over Na₂SO₄, and filtered. The residue was purified by silica gel column chromatography (EtOAc:MeOH=100:0 to 90:10) to obtain the yellow title compound (7 mg, 9.0 μmol, 7% yield, 91% purity).

¹H NMR (400 MHz, DMSO-d₆) δ=11.10 (s, 1H), 10.05 (s, 1H), 8.35 (s, 1H), 8.04 (brs, 1H), 7.84 (d, J=8.2 Hz, 1H), 7.62-7.50 (m, 4H), 7.17-7.13 (m, 1H), 7.04-7.01 (m, 1H), 6.91 (s, 1H), 6.62 (t, J=5.6 Hz, 1H), 5.03 (dd, J=12.8, 5.2 Hz, 1H), 4.12 (s, 2H), 3.69-3.65 (m, 8H), 3.51-3.47 (m, 4H), 2.86-2.76 (m, 4H), 2.60-2.54 (m, 4H), 1.92-1.87 (m, 4H).

MS[M+H]=773.2

### <Experimental Example 1> Measurement of IL-1 Activity by ELISA

### 1. Culture of THP-1 Cell Line

The THP-1 cell line was purchased from Korea Cell Line Bank. The passage in cell culture was maintained around P25.

For cell counting, Thermo's cell counter (Catalog #AMQAX1000) and 0.4% trypan blue solution were used.

For cell culture, RPMI1640 (Gibco, Cat. No. 22400-071; Lot. No. 2362356), Sodium pyruvate (Gibco, Cat. No. 11360-070; Lot. No. 2193075), HEPES (Gibco, Cat. No. 15630-080; Lot No. 2192573), MEM-NEAA (Gibco, Cat. No. 11140-050; Lot. No. 2269523), β-mercaptoethanol (Biorad, Cat. No. 1610710), penicillin/streptomycin (PS) (Gibco, Cat. No. 15140-122; Lot. No. 2211099), 75T cell culture flask (SPL, Cat. No. 70075), 96 well culture plate (SPL, Cat. No. 30096), PBS pH7.4 (Gibco, Cat. No. 10010-023; Lot. No. 2085080), Counting chamber (Hematocytometer) (Hirschmann, Cat. No. 8100204), and 0.4% trypan blue solution (DYNEBIO, Cat. No. CBT3710; Lot. No. 20190723) were used.

2. Treatment with Compound of the Present Invention

The cultured THP-1 cell lines were seeded at a density of 0.5×10⁵ cells for each well of a 96 well plate (SPL), and the cells were cultured in the culture medium with a total volume of 0.15 mL.

Prior to the treatment with the compound, the cells were treated with LPSO111 (Sigma, Cat. L4391; Lot No. 110081) to a final concentration of 1 μg/mL, and incubated at 37° C. for 4 hours.

The compound was completely dissolved in DMSO (Sigma, Cat. No. D2438; Lot. No. RNBJ9566) and used in the experiment, and treated by 3-fold dilution from the highest concentration of 100 nM, wherein the concentration of DMSO treated in each well was unified to 0.3%.

Finally, in 1 hour after the treatment with the compound, the cells were incubated with Nigericin (Sigma, Cat. No. N7143; Lot. No. 079M4051V) at a concentration of 12.5 μg/mL at 37° C. for 1 hour.

The cells and the medium were separated using a centrifuge, and then only the medium was stored and used in subsequent ELISA experiments.

### 3. Enzyme-Linked Immunosorbent Assay (ELISA)

To proceed with enzyme-linked immunosorbent assay, antibody was first coated on an immune plate (SPL, Cat. No. 32296; Lot. No. BA9E20A32296). Human IL-1β ELISA Kit (R&D systems, Cat. No. DY201; Lot. No. P288577) was used as the required reagent for each process. The antibody was diluted to 33.3 μg/mL and added to each well, followed by incubation at room temperature for more than 18 hours.

After washing each well three times with 0.05% Tween20 (Biorad, Cat. No. 1610781; Lot. No. 64399445) and 10% PBS (Gibco, Cat. No. 10010-023; Lot. No. 2306394) buffer, 1% BSA (SigmaAldrich, Cat. No. A3311; Lot. No. SLCF0913) PBS buffer was added, followed by incubation at room temperature for 1 hour. After removing the buffer, the storage medium was diluted 1/25 and added to each well, followed by incubation at room temperature for 2 hours.

After removing the medium, the cells were washed three times using 0.05% Tween20 and 10% PBS buffer. The detection antibody was diluted to 200 ng/mL and added to each well, followed by incubation at room temperature for 2 hours. After washing with 0.05% Tween20 and 10% PBS buffer, the substrate-containing Streptavidin-HRP was diluted to 1/40 and added to each well, followed by incubation at room temperature for 20 minutes after light blocking.

Finally, after washing three times with 0.05% Tween20 and 10% PBS buffer, 100 μL of ultra TMB (Thermo, Cat. No. 34028; Lot. No. WB3207922) was added to each well, followed by incubation for 15 minutes. For analysis of the results, final luminescence values were obtained using a microplate reader image (BMG labtech).

As a result of the experiment, it was confirmed that the activity of IL-1β was effectively reduced when THP-1 cells were treated with the compounds according to Examples of the present invention, thereby confirming that the NLRP3 PROTAC compound of the present invention is effective in NLRP3 inflammasome-related diseases.

1. A compound represented by the following Formula I, a stereoisomer thereof or a pharmaceutically acceptable salt thereof:
ULM-Linker-NTM [Formula I]
in Formula I above,
NTM is an NLRP3 (NACHT, LRR and PYD domains-containing protein 3) protein binding moiety,
ULM is a CRBN or VHL E3 ubiquitin ligase binding moiety, and
Linker is a group that chemically connects ULM and NTM.

2. The compound, the stereoisomer thereof or the pharmaceutically acceptable salt thereof according to claim 1, wherein NTM binds to the Walker A or B site in the NACHT domain of the NLRP3 protein.

3. The compound, the stereoisomer thereof or the pharmaceutically acceptable salt thereof according to claim 2, wherein NTM is an NLRP3 protein binding moiety represented by the following Formula N-1: 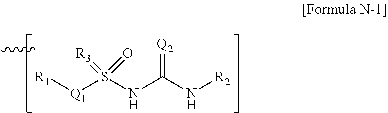
in Formula N-1 above,
R₁ and R₂ are each independently an optionally substitutable cycloalkyl, heterocyclyl, aryl or heteroaryl;
R₃ is O or NR₄;
R₄ is hydrogen, halogen, OH, NH₃, NO₂, CN, C₁₋₆alkyl, C₁₋₆alkoxy, C₂₋₆alkenyl, C₂₋₆alkynyl, 3-10 membered cycloalkyl, 4-10 membered heterocyclyl, 6-10 membered aryl or 4-10 membered heteroaryl; or R₄ and R₁ together with the atoms to which they are attached form an optionally substitutable 5 to 10 membered heterocyclic ring;
Q₁ is a single bond,-NQₓ-, -CQₓQ_{y}-, —CH₂-NQₓ- or —CH₂-CQₓQ_{y}- {wherein Qₓ and Q_{y} are each independently hydrogen or C₁₋₆alkyl};
Q₂ is O or S, and [symbol]
represents that any one hydrogen in the Formula N-1 is substituted with a single bond and connected to the Linker by a covalent bond.

4. The compound, the stereoisomer thereof or the pharmaceutically acceptable salt thereof according to claim 3, wherein the Formula N-1 is an NLRP3 protein binding moiety represented by the following Formula N-2: 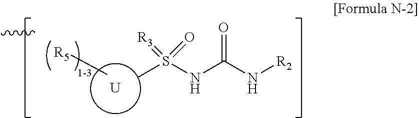
in Formula N-2 above,
\textcircled{U} is 4-10 membered cycloalkyl, 4-10 membered heterocyclyl, 6-10 membered aryl or 4-10 membered heteroaryl {wherein the heterocyclyl or heteroaryl comprises 1 to 3 N, S or O atoms};
R₂ is 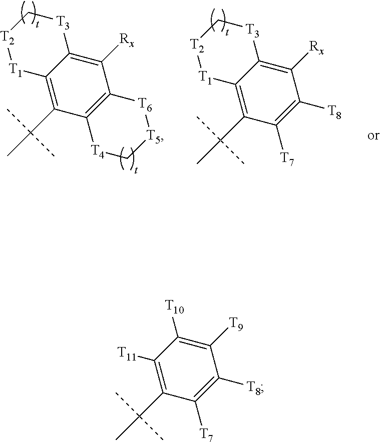
t is 0 or 1;
T₁ to T₆ are each independently —CH₂—, —NH—, —S—, —SO₂— or —O— {wherein hydrogen in T₁ to T₆ may each independently be substituted with C₁₋₆alkyl, halogen, OH, OCH₃, NH₂ or CN};
T₇ to T₁₁ are each independently C₁₋₆alkyl, halogen, OH, OCH₃, CN, 4-10 membered cycloalkyl, 4-10 membered heterocyclyl, 6-10 membered aryl or 4-10 membered heteroaryl {wherein the heterocyclyl or heteroaryl comprises 1 to 3 N, S or O atoms};
hydrogen in T₇ to T₁₁ may each independently be substituted with C₁₋₆alkyl, halogen, OH, OCH₃, NH₂ or CN;
Rₓ is hydrogen, halogen, C₁₋₆alkyl, O(C₁₋₄alkyl), OH, CN, NH₃, NO₂, SO₂ or CN;
R₃ is O or NR₄;
R₄ is hydrogen, halogen, OH, OCH₃, CN or C₁₋₃alkyl;
R₅ is each independently —(C₀₋₄alkylene)-R₆, —(C₀₋₄alkylene)-R_{L1}—(C₀₋₄alkylene)-R₆ or —(C₀₋₄alkylene)-R_{L1}—(C₀₋₄alkylene)-R_{L2}—(C₀₋₄alkylene)-R₆;
R₆ is hydrogen, halogen, OH, OCH₃, COH, COOH, CN, NH₂, NH(C₁₋₃alkyl), NCH₃(C₁₋₃alkyl), SO₂, C₁₋₆alkyl, C₂₋₆alkenyl, C₂₋₆alkynyl, cycloalkyl, heterocyclyl, aryl or heteroaryl {wherein at least one hydrogen in R₆ may each independently be substituted with halogen, C₁₋₃alkyl, C₁₋₃alkoxy, OH, NH₂, NO₂ or CN};
R_{L1} and R_{L2} are each independently —O—, —CO—, —COO—, —OCO—, —NH—, —N(C₁₋₃alkyl)-, —NHCO—, —N(C₁₋₃alkyl)CO—, —CONH—, —CON(C₁₋₃alkyl)- or —NHCONH—, and [symbol]
represents that any one hydrogen in the Formula N-2 is substituted with a single bond and connected to the Linker by a covalent bond.

5. The compound, the stereoisomer thereof or the pharmaceutically acceptable salt thereof according to claim 4, wherein the Formula N-2 is an NLRP3 protein binding moiety represented by the following Formula N-3: 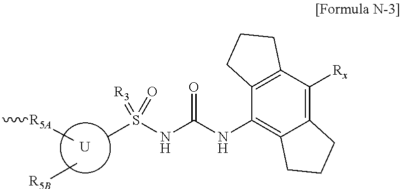
in Formula N-3 above,
R₃ is O, NH or N—CN; 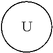
is 5-7 membered cycloalkyl, 5-7 membered heterocyclyl, phenyl or 5-6 membered heteroaryl {wherein the heterocyclyl or heteroaryl comprises 1 to 3 N, S or O atoms};
R_{5A} and R_{5B} are each independently —(C₀₋₄alkylene)-R₆ or —(C₀₋₄alkylene)-R_{L}—(C₀₋₄alkylene)-R₆;
R₆ is hydrogen, halogen, OH, OCH₃, COH, COOH, CN, NH₂, NH(C₁₋₃alkyl), NCH₃(C₁₋₃alkyl), SO₂, C₁₋₆alkyl, C₂₋₆alkenyl, C₂₋₆alkynyl, 4-8 membered cycloalkyl, 4-8 membered heterocyclyl, phenyl or 5-6 membered heteroaryl {wherein the heterocyclyl or heteroaryl comprises 1 to 3 N, S or O atoms, and hydrogen in R₆ may be substituted with C₁₋₃alkyl, OH, —O(C₁₋₃alkyl), CN, halogen};
R_{L} is —O—, —CO—, —COO—, —OCO—, —NH—, —N(C₁₋₃alkyl)-, —NHCO—, —N(C₁₋₃alkyl)CO—, —CONH—, —CON(C₁₋₃alkyl)- or —NHCONH—;
Rₓ is hydrogen, halogen, OH or CN, and [symbol]
represents that any one hydrogen in R_{5A} is substituted with a single bond and connected to the Linker by a covalent bond.

6. The compound, the stereoisomer thereof or the pharmaceutically acceptable salt thereof according to claim 1,
wherein ULM is a CRBN E3 ubiquitin ligase binding moiety represented by the following Formula A-1: 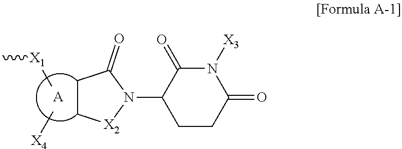
in Formula A-1 above, 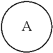
is a ring selected from 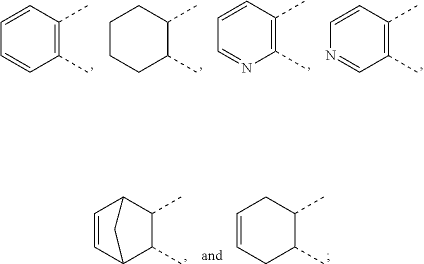
X₁ is a single bond, —CH₂—, —NH—, —O—, —CH₂CH₂—, —CC—CO—, —COO—, —NHCO— or —CONH—;
X₂ is —CH₂—, —CH(C₁₋₄alkyl)-, —NH—, —N(C₁₋₄alkyl)-, —O—, —CO—, —CH₂—CH₂—, —NH—CH₂—, —NH—CH(C₁₋₄alkyl)-, —N═CH—, —N═C(C₁₋₄alkyl)- or —N═N—;
X₃ is hydrogen or C₁₋₄alkyl;
X₄ is hydrogen, halogen, C₁₋₆alkyl, CN, NH₂, NO₂, OH, COH, COOH or CF₃; and [symbol]
represents that the Linker is covalently linked to the moiety represented by Formula A-1 above.

7. The compound, the stereoisomer thereof or the pharmaceutically acceptable salt thereof according to claim 6,
wherein ULM is a CRBN E3 ubiquitin ligase binding moiety represented by the following Formula A-2: 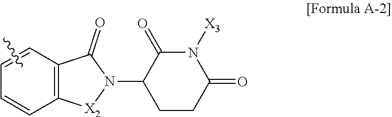
in Formula A-2 above,
X₂ is —CH₂—, —CH(C₁₋₄alkyl)-, —CO— or —N═N—;
X₃ is hydrogen or C₁₋₃alkyl; and [symbol]
represents that the Linker is covalently linked to the moiety represented by Formula A-2 above.

8. The compound, the stereoisomer thereof or the pharmaceutically acceptable salt thereof according to claim 1,
wherein ULM is a VHL E3 ubiquitin ligase binding moiety represented by the following Formula B-1: 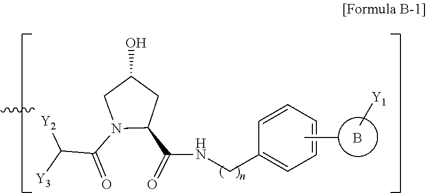
in Formula B-1 above,
n is an integer from 1 to 3;
\textcircled{B} is 5-6 membered cycloalkyl, phenyl, 5-6 membered heterocyclyl or 5-6 membered heteroaryl {wherein the heterocyclyl or heteroaryl comprises 1 to 3 N, O or S atoms};
Y₁ is hydrogen or C₁₋₄alkyl;
Y₂ is C₁₋₄alkyl, hydroxy(C₁₋₄alkyl), —(C₀₋₂alkyl)-COH, C₃₋₈cycloalkyl, or phenyl;
Y₃ is hydrogen or 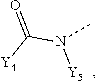
Y₄ is hydrogen, halogen, C₁₋₄alkyl, —O(C₁₋₄alkyl), C₃₋₆cycloalkyl or 4-6 membered heterocyclyl {wherein hydrogen in Y₄ may be substituted with halogen, —OH, —CN, —NHCOH, —NHCOCH₃, —COH or —COCH₃};
Y₅ is hydrogen or C₁₋₄alkyl; and [symbol]
represents that the Linker is covalently linked to the moiety represented by Formula B-1 above.

9. The compound, the stereoisomer thereof or the pharmaceutically acceptable salt thereof according to claim 8,
wherein ULM is a VHL E3 ubiquitin ligase binding moiety represented by the following Formula B-2: 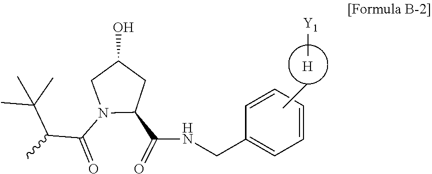
in Formula B-2 above, 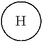
is a 5-membered heteroaryl ring selected from oxazole, isoxazole, thiazole, isothiazole, imidazole, pyrazole, triazole, oxadiazole, pyrrole, pyrrolidine, furan, dihydrofuran and tetrahydrofuran;
Y₁ is hydrogen or C₁₋₃alkyl; and [symbol]
represents that the Linker is covalently linked to the moiety represented by Formula B-2 above.

10. The compound, the stereoisomer thereof or the pharmaceutically acceptable salt thereof according to claim 1,
wherein the Linker is a chemical group represented by the following Formula L: 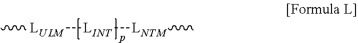
in Formula L above, [symbol]
and [symbol]
are bonds;
L_{ULM} binds to the ULM moiety through [symbol]
linked thereto;
L_{NTM} binds to the NTM moiety through [symbol]
linked thereto;
L_{ULM}, L_{NTM} and L_{INT} are each independently selected from a single bond, —CH₂—, —NH—, —O—, —S—, —SO—, —SO₂—, —CO—, —CH₂CH₂—, —CHCH—, —CC—, —CH₂CH₂O—, —OCH₂CH₂—, —CH₂CH₂S—, —SCH₂CH₂—, —COO—, —CONH—, —NHCO—, and 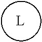
{wherein 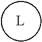
is cycloalkyl, heterocyclyl, aryl or heteroaryl};
L_{ULM}, L_{NTM} and L_{INT} may each independently be substituted with at least one C₁₋₆alkyl, C₃₋₈cycloalkyl, halogen, hydroxy, amine, nitro, cyano or haloalkyl; and
p is an integer from 1 to 30.

11. The compound, the stereoisomer thereof or the pharmaceutically acceptable salt thereof according to claim 10,
wherein L_{ULM} is 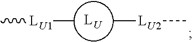
L_{U1} is selected from a single bond, —CH₂—, —CH₂CH₂—, —CH═CH—, —CC—, —NH—, —NCH₃—, —CO—, —NHCO—, and —O—;
L_{U2} is selected from a single bond, —CH₂—, —NH—, —O—, —CO—, and —CONH—; and 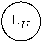
is a single bond, C₁₋₆alkyl, or a ring selected from 3-10 membered cycloalkyl, 4-10 membered heterocyclyl, 6-10 membered aryl, and 5-10 membered heteroaryl.

12. The compound, the stereoisomer thereof or the pharmaceutically acceptable salt thereof according to claim 10, wherein L_{NTM} is 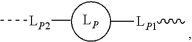
L_{P1} is selected from a single bond, —O—, —S—, —NH—, —N(C₁₋₄alkyl)-, —CH₂—, —CH(C₁₋₄alkyl)-, —CH₂NH—, and —CH₂CH₂—,
L_{P2} is selected from a single bond, —CO—, —COCH₂—, —NHCO—, —NHCOCH₂—, -HET- and -HET-CH₂—, wherein HET is 5-6 membered heterocyclyl or heteroaryl having at least one N, S or O atom, and 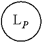
is a single bond, C₁₋₈alkyl substituted with amine group; or a ring selected from 3-10 membered cycloalkyl, 4-10 membered heterocyclyl, 6-10 membered aryl and 5-10 membered heteroaryl.

13. The compound, the stereoisomer thereof or the pharmaceutically acceptable salt thereof according to claim 10,
wherein 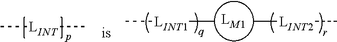 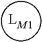
is a single bond; or a ring selected from 3-10 membered cycloalkyl, 4-10 membered heterocyclyl, 6-10 membered aryl and 5-10 membered heteroaryl,
L_{INT1} and L_{INT2} are each independently selected from —CH₂—, —NH—, —NCH₃—, —O—, —S—, —SO—, —SO₂—, —CO—, —CH₂CH₂O—, —OCH₂CH₂—, —CH₂CH₂S—, —SCH₂CH₂—, —COO—, —CONH— and —NHCO—, and
q and r are each independently an integer of 1 to 10.

14. The compound, the stereoisomer thereof or the pharmaceutically acceptable salt thereof according to claim 1, wherein the compound is listed in Table below:

| Compound | Structure |
|---|---|
| 1 | 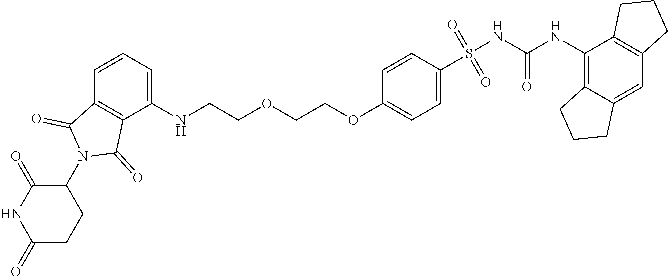 [SMILES: O=C1CCC(N2C(=O)c3cccc(NCCOCCOc4ccc(S(=O)(=O)NC(=O)Nc5c6c(cc7c5CCC7)CCC6)cc4)c3C2=O)C(=O)N1] |
| 2 | 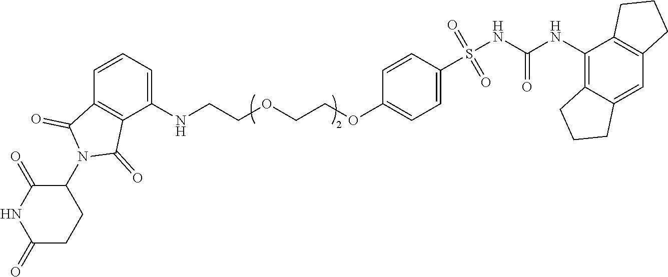 |
| 3 | 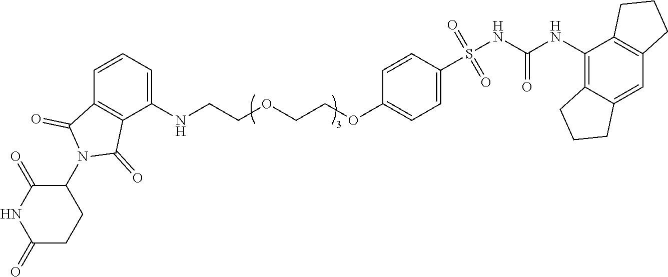 |
| 4 | 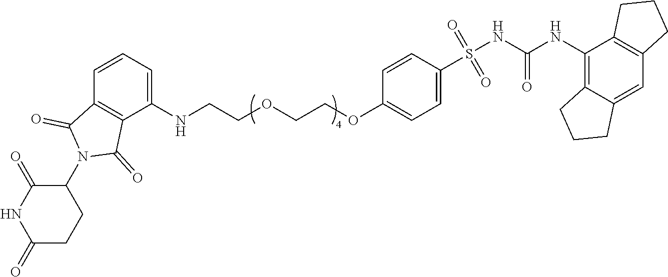 |
| 5 | 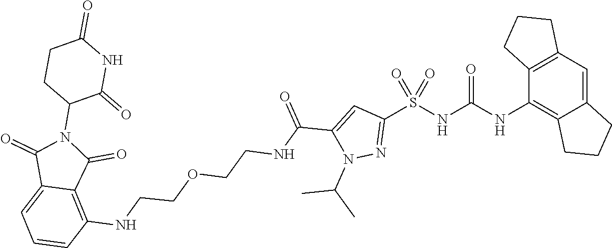 [SMILES: CC(C)n1nc(S(=O)(=O)NC(=O)Nc2c3c(cc4c2CCC4)CCC3)cc1C(=O)NCCOCCNc1cccc2c1C(=O)N(C1CCC(=O)NC1=O)C2=O] |
| 6 | 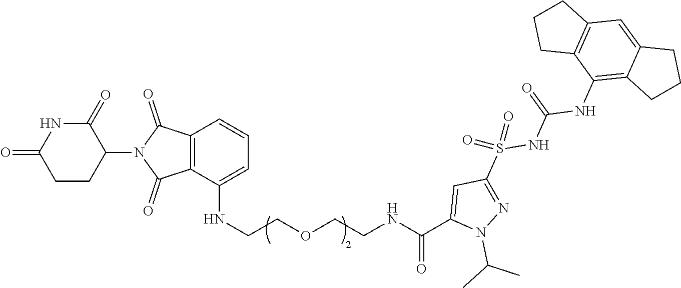 |
| 7 | 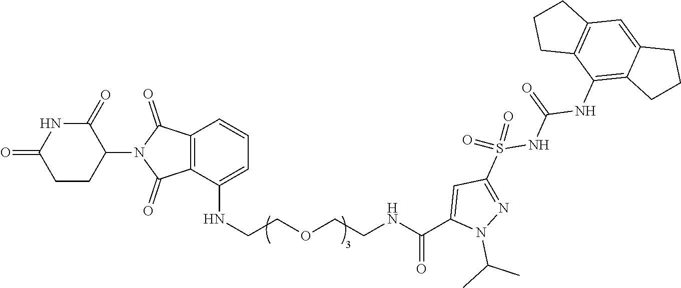 |
| 8 | 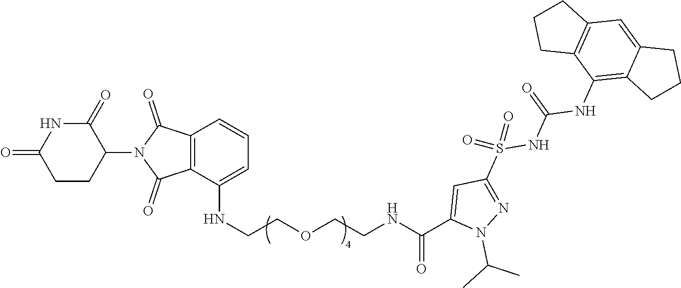 |
| 9 | 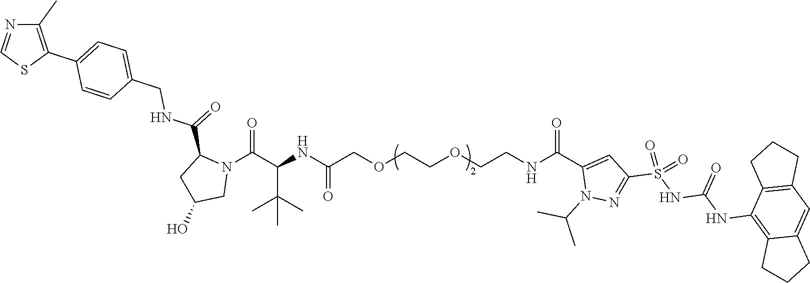 |
| 10 | 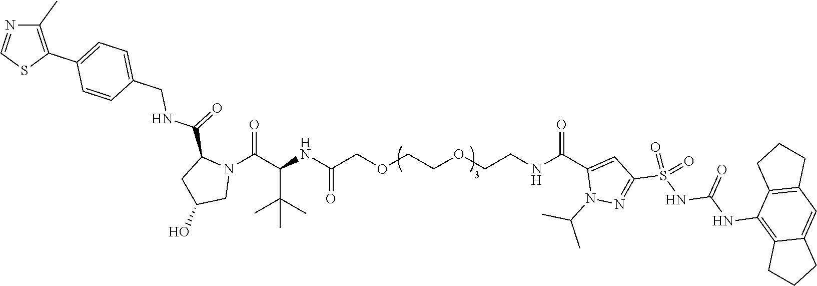 |
| 11 | 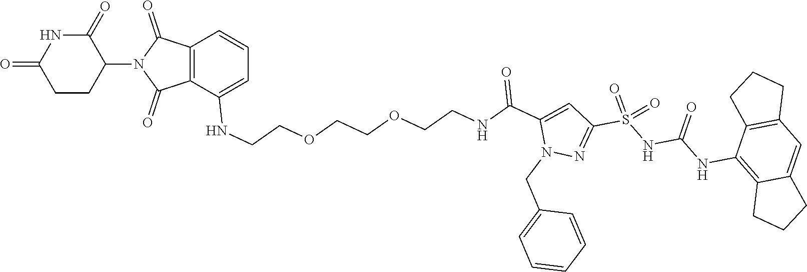 [SMILES: O=C1CCC(N2C(=O)c3cccc(NCCOCCOCCNC(=O)c4cc(S(=O)(=O)NC(=O)Nc5c6c(cc7c5CCC7)CCC6)nn4Cc4ccccc4)c3C2=O)C(=O)N1] |
| 12 | 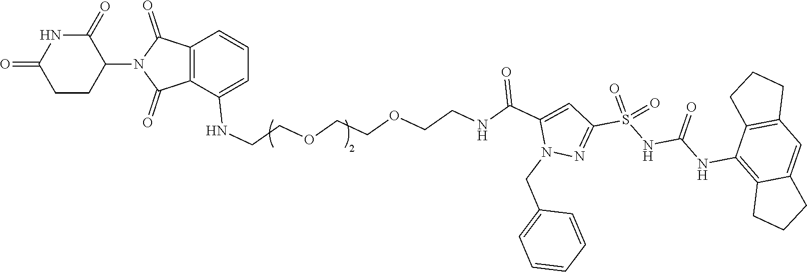 |
| 13 | 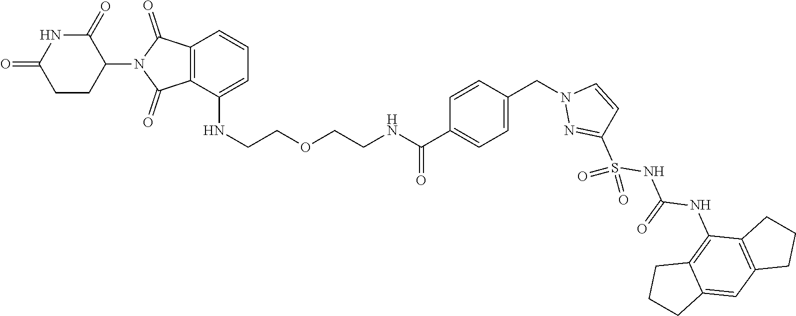 [SMILES: O=C1CCC(N2C(=O)c3cccc(NCCOCCNC(=O)c4ccc(Cn5ccc(S(=O)(=O)NC(=O)Nc6c7c(cc8c6CCC8)CCC7)n5)cc4)c3C2=O)C(=O)N1] |
| 14 | 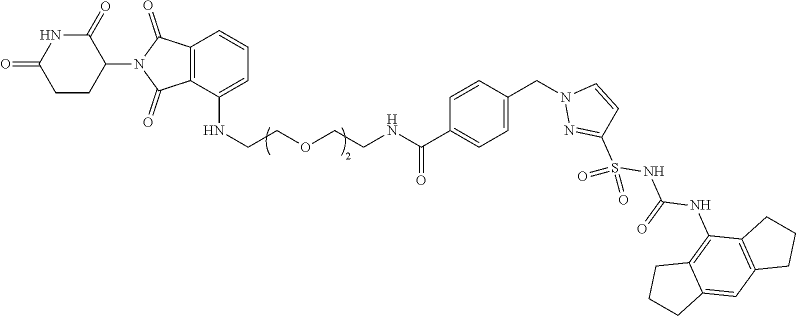 |
| 15 | 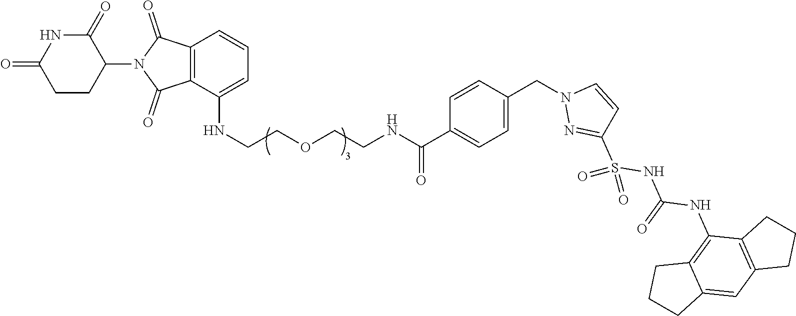 |
| 16 | 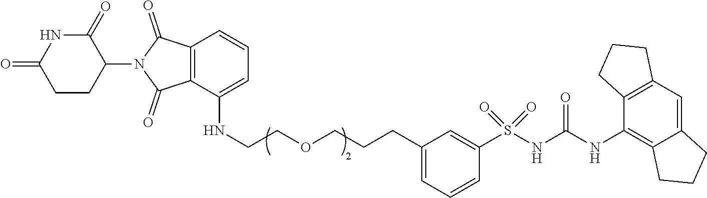 |
| 17 | 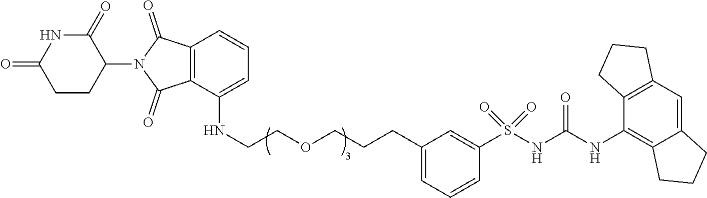 |
| 18 | 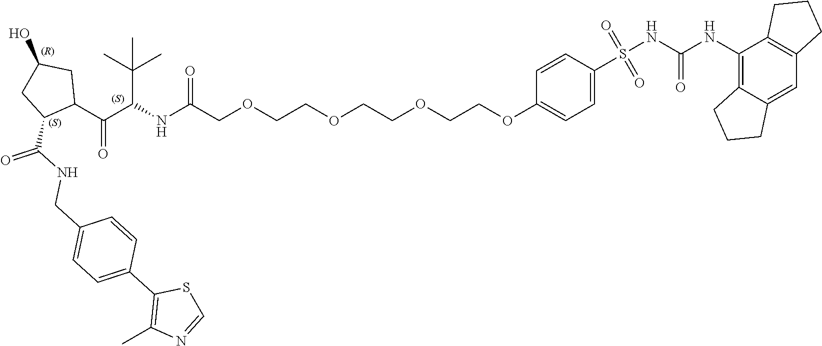 |
| 19 | 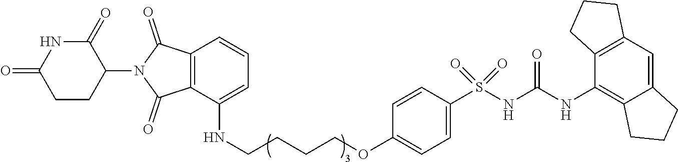 |
| 20 | 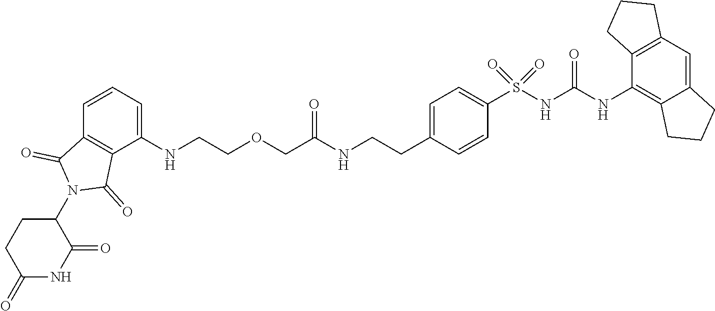 [SMILES: O=C(COCCNc1cccc2c1C(=O)N(C1CCC(=O)NC1=O)C2=O)NCCc1ccc(S(=O)(=O)NC(=O)Nc2c3c(cc4c2CCC4)CCC3)cc1] |
| 21 | 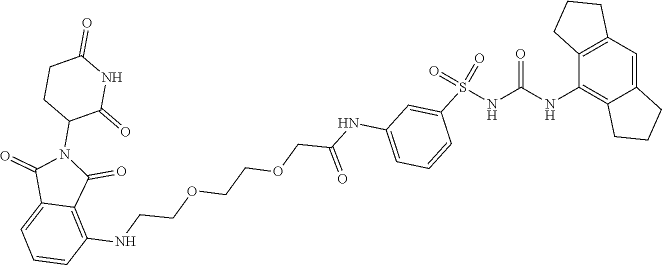 [SMILES: O=C1CCC(N2C(=O)c3cccc(NCCOCCOCC(=O)Nc4cccc(S(=O)(=O)NC(=O)Nc5c6c(cc7c5CCC7)CCC6)c4)c3C2=O)C(=O)N1] |

15. A pharmaceutical composition for degrading NLRP3 protein comprising the compound, the stereoisomer thereof or the pharmaceutically acceptable salt thereof of claim 1.

16. The pharmaceutical composition according to claim 15, wherein the pharmaceutical composition further comprises at least one type of pharmaceutically acceptable carrier.

17. The pharmaceutical composition according to claim 15, wherein the pharmaceutical composition is for preventing or treating NLRP3 inflammasome-related diseases.

18. The pharmaceutical composition according to claim 17, wherein the NLRP3 inflammasome-related diseases are selected from central nervous system diseases, metabolic disorders, cardiovascular diseases, respiratory diseases, liver diseases, pancreatic diseases, kidney diseases, bowel diseases, skin diseases, musculoskeletal diseases, bone diseases, ocular diseases, inflammation after viral infection, autoimmune diseases, cancer or tumor, and inflammatory diseases.

19. A method of preventing or treating NLRP3 inflammasome-related diseases comprising administering to patients a therapeutically effective amount of the compound of claim 1.
